SEQUENCE LISTING (40 sequences)

SEQ ID NO: 1 - DNA, 4779 bp, synthetic construct
  source 1..4779: mol_type = other DNA
atggctcccaagaagaagcggaaggtcgagggcatcaagagcaacatcagcctgctgaaggacgagctgagaggccagatcagccacatctcccacgagtacctgagcctgatcgacctggccttcgaccccaagcagaaccggctgttcgagatgaaggtgctggaactgctggtcaacgagtacggcttcaagggcagacacctcggcggcagcagaaagcctgatggcatcgtgtacagcaccacactcgaggacaacttcggcatcatcgtggacaccaaggcctacagcgagggctacagcctgcctatctctcaggccgacgagatggaaagatacgtgcgcgagaacagcaaccgcgacgaggaagtgaaccccaacaagtggtgggagaacttcagcgaggaagtcaaaaagtactacttcgtgttcatcagcggcagctttaagggcaagttcgaggaacagctgcggcggctgtctatgaccacaggcgttaacggcagcgccgtgaacgtggtcaatctgctgctgggcgccgagaagattagaagcggcgagatgaccatcgaggaactggaacgggccatgttcaacaacagcgagttcatcctgaagtacggcggaggcggcagcgacaagaagtactctatcggactggccatcggcaccaactctgttggatgggccgtgatcaccgacgagtacaaggtgcccagcaagaaattcaaagtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggacaagaagcacgagcggcaccccatcttcggaaatatcgtggacgaggtggcctaccacgagaagtaccccaccatctaccacctgagaaagaaactggtggacagcaccgacaaggccgacctgcgactgatctatctggccctggctcacatgatcaagttccggggccacttcctgatcgagggcgacctgaatcctgacaactccgacgtggacaagctgttcatccagctggtgcagacctacaatcagctgttcgaagagaatcccatcaacgcctctggcgtggacgccaaagccatcctgtctgccagactgagcaagagcagacggctggaaaacctgatcgctcagctgcccggcgagaagaagaatggcctgttcggcaacctgattgccctgtctctgggcctgacacctaacttcaagtccaacttcgatctggccgaggatgccaaactgcagctgtccaaggacacctacgacgacgacctggataacctgctggcccagatcggcgatcagtacgccgacttgtttctggccgccaagaacctgtctgacgccatcctgctgagcgacatcctgagagtgaacaccgagatcacaaaggcccctctgagcgcctctatgatcaagagatacgacgagcaccaccaggatctgaccctgctgaaagctctcgtcaggcagcagctgccagagaagtacaaagagattttcttcgaccagagcaagaacggctacgccggctacattgatggcggagccagccaagaggaattctacaagttcatcaagcccatcctcgagaagatggacggcacagaggaactgctcgtgaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaagaggtggtggacaagggcgccagcgctcagtccttcatcgagcggatgaccaatttcgacaagaatctgcccaacgagaaagtgctgcccaagcactccctgctgtacgagtacttcaccgtgtacaacgagctgaccaaagtgaaatacgtgaccgagggaatgagaaagcccgcctttctgtccggcgagcagaaaaaggccatcgtggatctgctgttcaagaccaaccggaaagtgaccgtgaagcagctgaaagaggactacttcaagaaaatcgagtgcttcgactccgtggaaatcagcggcgtggaagatcggttcaatgccagcctgggcacataccacgatctgctgaaaattatcaaggacaaggacttcctggacaacgaggaaaacgaggacatccttgaggacatcgtgctgaccctgacactgttcgaggacagagagatgatcgaggaaaggctgaaaacatacgcccacctgttcgacgacaaagtcatgaagcaactgaagcggcggcgctacacaggctggggcagactgtctagaaagctgatcaacggcatccgggacaagcagtccggcaagaccatcctggactttctgaagtccgacggcttcgccaacagaaacttcatgcagctgattcacgacgacagcctcaccttcaaagaggacattcagaaggcccaggtttccggccagggcgattctctgcacgagcacattgccaatctggccggctctcccgccattaagaagggcattctgcagacagtgaaagtggtggatgagctggtcaaagtgatggggagacacaagcccgagaacatcgtgatcgaaatggccagagagaaccagaccacacagaagggccagaagaactcccgcgagagaatgaagcggatcgaagagggaatcaaagagctggggagccagatcctgaaagaacaccccgtggaaaacacccagctgcagaacgagaagctgtacctgtactacctccagaacggccgggatatgtacgtggaccaagagctggacatcaaccgcctgagcgactacgatgtggacgctatcgtgccccagtcttttctgaaagatgactccatcgacaacaaggtgctgaccagaagcgataagaaccggggcaagagcgacaacgtgccctctgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacaccaaatacgatgagaacgacaaactgatccgggaagtgaaagtcatcaccctgaagtccaagctggtgtccgatttccggaaggatttccagttctacaaagtgcgggaaatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggaaaggctaccgccaaatacttcttctactccaacatcatgaactttttcaagacagagatcaccctcgccaacggcgagatcagaaagcggcctctgatcgagacaaacggcgaaaccggcgagattgtgtgggataagggcagagactttgccacagtgcggaaggtgctcagcatgccccaagtgaatatcgtgaaaaagaccgaggtgcagacaggcggcttcagcaaagagtccattctgcctaagcggaactccgacaagctgatcgcccggaagaaggactgggaccccaagaaatacggcggcttcgatagccctaccgtggcctattctgtgctggtggtggccaaagtggaaaagggaaagtccaagaagctcaagagcgtcaaagaactcctgggcatcaccatcatggaacggtccagcttcgagaagaaccctatcgactttctggaagccaagggctacaaagaagtcaagaaggacctgatcatcaagctccccaagtacagcctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcagaagcagcttttcgtcgagcagcacaagcactacctggacgagatcatcgagcagatctccgagttctccaagagagtgatcctggccgacgccaacctggacaaggttctgtccgcctacaacaagcaccgggataagcccatcagagagcaggccgagaatatcatccacctgtttaccctgaccaacctgggagcccctgccgccttcaagtacttcgacaccaccatcgaccggaagcgctacaccagcaccaaagaagtgctggacgccacactgatccaccagagcatcaccggcctgtacgagacacggatcgatctgtctcagcttggaggcgacggcagccctaagaagaagagaaaggtttccagctaataa

SEQ ID NO: 2 - RNA, 5124 nt, synthetic construct
  source 1..5124: mol_type = other RNA
acatttgcttctgacacaactgtgttcactagcaacctcaaacagacaccaagcttgccaccatggctcccaagaagaagcggaaggtcgagggcatcaagagcaacatcagcctgctgaaggacgagctgagaggccagatcagccacatctcccacgagtacctgagcctgatcgacctggccttcgaccccaagcagaaccggctgttcgagatgaaggtgctggaactgctggtcaacgagtacggcttcaagggcagacacctcggcggcagcagaaagcctgatggcatcgtgtacagcaccacactcgaggacaacttcggcatcatcgtggacaccaaggcctacagcgagggctacagcctgcctatctctcaggccgacgagatggaaagatacgtgcgcgagaacagcaaccgcgacgaggaagtgaaccccaacaagtggtgggagaacttcagcgaggaagtcaaaaagtactacttcgtgttcatcagcggcagctttaagggcaagttcgaggaacagctgcggcggctgtctatgaccacaggcgttaacggcagcgccgtgaacgtggtcaatctgctgctgggcgccgagaagattagaagcggcgagatgaccatcgaggaactggaacgggccatgttcaacaacagcgagttcatcctgaagtacggcggaggcggcagcgacaagaagtactctatcggactggccatcggcaccaactctgttggatgggccgtgatcaccgacgagtacaaggtgcccagcaagaaattcaaagtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggacaagaagcacgagcggcaccccatcttcggaaatatcgtggacgaggtggcctaccacgagaagtaccccaccatctaccacctgagaaagaaactggtggacagcaccgacaaggccgacctgcgactgatctatctggccctggctcacatgatcaagttccggggccacttcctgatcgagggcgacctgaatcctgacaactccgacgtggacaagctgttcatccagctggtgcagacctacaatcagctgttcgaagagaatcccatcaacgcctctggcgtggacgccaaagccatcctgtctgccagactgagcaagagcagacggctggaaaacctgatcgctcagctgcccggcgagaagaagaatggcctgttcggcaacctgattgccctgtctctgggcctgacacctaacttcaagtccaacttcgatctggccgaggatgccaaactgcagctgtccaaggacacctacgacgacgacctggataacctgctggcccagatcggcgatcagtacgccgacttgtttctggccgccaagaacctgtctgacgccatcctgctgagcgacatcctgagagtgaacaccgagatcacaaaggcccctctgagcgcctctatgatcaagagatacgacgagcaccaccaggatctgaccctgctgaaagctctcgtcaggcagcagctgccagagaagtacaaagagattttcttcgaccagagcaagaacggctacgccggctacattgatggcggagccagccaagaggaattctacaagttcatcaagcccatcctcgagaagatggacggcacagaggaactgctcgtgaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaagaggtggtggacaagggcgccagcgctcagtccttcatcgagcggatgaccaatttcgacaagaatctgcccaacgagaaagtgctgcccaagcactccctgctgtacgagtacttcaccgtgtacaacgagctgaccaaagtgaaatacgtgaccgagggaatgagaaagcccgcctttctgtccggcgagcagaaaaaggccatcgtggatctgctgttcaagaccaaccggaaagtgaccgtgaagcagctgaaagaggactacttcaagaaaatcgagtgcttcgactccgtggaaatcagcggcgtggaagatcggttcaatgccagcctgggcacataccacgatctgctgaaaattatcaaggacaaggacttcctggacaacgaggaaaacgaggacatccttgaggacatcgtgctgaccctgacactgttcgaggacagagagatgatcgaggaaaggctgaaaacatacgcccacctgttcgacgacaaagtcatgaagcaactgaagcggcggcgctacacaggctggggcagactgtctagaaagctgatcaacggcatccgggacaagcagtccggcaagaccatcctggactttctgaagtccgacggcttcgccaacagaaacttcatgcagctgattcacgacgacagcctcaccttcaaagaggacattcagaaggcccaggtttccggccagggcgattctctgcacgagcacattgccaatctggccggctctcccgccattaagaagggcattctgcagacagtgaaagtggtggatgagctggtcaaagtgatggggagacacaagcccgagaacatcgtgatcgaaatggccagagagaaccagaccacacagaagggccagaagaactcccgcgagagaatgaagcggatcgaagagggaatcaaagagctggggagccagatcctgaaagaacaccccgtggaaaacacccagctgcagaacgagaagctgtacctgtactacctccagaacggccgggatatgtacgtggaccaagagctggacatcaaccgcctgagcgactacgatgtggacgctatcgtgccccagtcttttctgaaagatgactccatcgacaacaaggtgctgaccagaagcgataagaaccggggcaagagcgacaacgtgccctctgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacaccaaatacgatgagaacgacaaactgatccgggaagtgaaagtcatcaccctgaagtccaagctggtgtccgatttccggaaggatttccagttctacaaagtgcgggaaatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggaaaggctaccgccaaatacttcttctactccaacatcatgaactttttcaagacagagatcaccctcgccaacggcgagatcagaaagcggcctctgatcgagacaaacggcgaaaccggcgagattgtgtgggataagggcagagactttgccacagtgcggaaggtgctcagcatgccccaagtgaatatcgtgaaaaagaccgaggtgcagacaggcggcttcagcaaagagtccattctgcctaagcggaactccgacaagctgatcgcccggaagaaggactgggaccccaagaaatacggcggcttcgatagccctaccgtggcctattctgtgctggtggtggccaaagtggaaaagggaaagtccaagaagctcaagagcgtcaaagaactcctgggcatcaccatcatggaacggtccagcttcgagaagaaccctatcgactttctggaagccaagggctacaaagaagtcaagaaggacctgatcatcaagctccccaagtacagcctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcagaagcagcttttcgtcgagcagcacaagcactacctggacgagatcatcgagcagatctccgagttctccaagagagtgatcctggccgacgccaacctggacaaggttctgtccgcctacaacaagcaccgggataagcccatcagagagcaggccgagaatatcatccacctgtttaccctgaccaacctgggagcccctgccgccttcaagtacttcgacaccaccatcgaccggaagcgctacaccagcaccaaagaagtgctggacgccacactgatccaccagagcatcaccggcctgtacgagacacggatcgatctgtctcagcttggaggcgacggcagccctaagaagaagagaaaggtttccagctaataaggcggccgccctcgccccggacctgccctcccgccaggtgcacccacctgcaataaatgcagcgaagccgggagaattccctcgccccggacctgccctcccgccaggtgcacccacctgcaataaatgcagcgaagccgggagcggccgcggatccccgggtaccgaattcgatatctctatagtgtcacctaaatttaattaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - protein, 1591 aa, synthetic construct
MAPKKKRKVEGIKSNISLLKDELRGQISHISHEYLSLIDLAFDPKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

SEQ ID NO: 4 - RNA, 20 nt, synthetic construct
  source 1..20: mol_type = other RNA
gatgaccattggtattggag

SEQ ID NO: 5 - RNA, 20 nt, synthetic construct
  source 1..20: mol_type = other RNA
gaataacatgcagactaagc

SEQ ID NO: 6 - DNA, 4651 bp, synthetic construct
  source 1..4651: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttggcggccgccctctccaataccaatggtcatccctaaaacatacgtatgaataacatgcagactaagcaggagagtagtccagggtttccgagggtttccgtcgactcagctcgtctcgagggcgtactaactgggccctttcccttttttttcctcagatgggttctggagctacaaacttcagcctcctcaagcaagctggagacgtggaagagaacccaggacctgaagacgctaagaacatcaagaagggacccgctcccttttaccctctggaggatgggaccgccggagaacagctccataaggctatgaaacggtatgcattggttcccggggctatagccttcaccgatgctcatattcaagtagacgtaacctatgcagagtacttcgagatgtcagtccggctcgcagaagcaatgcggcgatacggtttgaacacaaatcaccgcatagtcgtttgttcagaaaactcctctcaattttttatgcccgtactcggagcactctttattggtgttgcagtggcccccgctaacgacatatacaacgagagggagttgctgaactcaatgggaattagccaaccaacagtggtattcgtttcaaagaagggactgcgcaaagttcttaacgtacagaagaaattgcccataatccgaaaaatcattattatggactccaaaaccgactatcaggggtttcagagcatgtataccttcgtcacctctcaccttcctccctcattcaacgagtatgattttgttcccgaaagttttgaccgcgacaaaaccattgcactgatcatgaatagtagtgggagcacagggcttccaaaaggagttgctttgcctcaccgaacagcttgcgtgcggttcagccatgctagagaccctatctttggctaccagaacattcccgatacagccatcctcagtgtagtcccattccaccatggtttcggaatgtttactacactcggatatttgatttgcgggtttcgcgtcgtgctcatgtatcgcttcgaagaggagttgtttctccgctccctgcaagattacaaaattcaatctgccttgctggtgcctactcttttcagttgtctggccaagtcaacccttatcgacaagtacgatctctcatcattgcgggaaatagcctctggcggcgcaccactgtcaaaagaagtaggtgaggcagtagcaaagcgctttagattgccaggcatcaggcagggttatggtctgacagaaacaaccaatgctgttatgattactccagagggcgaccggaagccaggttctgtgggtaaagtagtccctttcttcgaggcaaaggtagtcgacttggtgacagggaagaccctcggcgtgaaccagcggggggaattgtgtgtacggggtcctatgatcatgagtggctatgtcaacaatcctgaagcaaccaatgctctcatcgataaggatggctggttgcatagtggtgatattgcttactgggatgaggacgagcacttttttattgttgataggctcaaatccctgatcaaatacaagggataccaggtggccccagcagaacttgagggaatactcctccagcacccatacattttcgatgctggagttgccggtttgccagacgatgatgcaggagagcttccagctgcagtggttgttcttgaacacggaaaaactatgacagagaaggagatcgtagattatgtagctagccaggtgactacagccaagaagctcagaggaggagtcgtattcgtagatgaagtaccacggggatcaacaggcaaactggatgcacggaaaatcagagagattttgaccaaagccaagaaggatggaaaaattgccgtgtagcaagtttgtacaaaaaagcaggctgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggccgaaccggccgatatcaagcttcgaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccaattctggcccgcattaccggtcatcctgcagcctacacggcgatctttccgtccttcttggccttagtgagaatctcgcggatcttgcgggcgtctaacttgccggtcgatcctctaggtacctcatccacaaacacaacaccaccgcgcagcttcttggcggttgtaacctggctggccacatagtccacgatctctttctcggtcatggttttaccgtgttccaacacgacgactgcggcgggcagctcgccggcatcgtcgtcgggcaggccggcgactccggcgtcgaagatgtaggggtgttgcagcaggatgccctccagttcggctggggctacctggtagcccttgtatttgatcagggacttcagccggtccacgatgaagaagtgctcgtcctcgtcccagtaggcgatgtcgccgctgtgcagccagccgtccttgtcgatgagagcgttcgtagcctcggggttgttaacgtagccgctcatgatcatggggccacggacgcacagctcaccgcgctggttcacacccagtgtcttacccgtgaccaagtctaccaccttagcctcgaagaagggcaccaccttgcctactgagccaggcttacggtccccctcgggggtgatcatgacggcgttggttgtttctgtcaggccatagccctggcggatgcctggtaggcggaagcgtttggccacggcctcacctacctccttgctgagcggcgccccgccgctggcgatctcgcgcaagctgcttaggtcgtacttgtcgatgagagtgctcttagcgaggcagctaaatagtgtgggcaccagcagggcagattgaatcttatagtcttgcaagctgcgcaagaatagctcctcctcgaagcggtacatgagcacgacccgaaagccgcagatcaagtagcccagcgtggtgaacatgccgaagccgtggtgaaatggcaccacgctgaggatagcggtgtcggggatgttctggtagccgaagatggggtcgcgggcatgactgaatcggacacaagcggtgcggtgcggtagggctacgcccttgggtaatcctgtactaccactactgttcatgatcagggcgatggttttgtcccggtcgaagctctcgggcacgaagtcgtactcgttgaagctgggtggcaaatgggaagtcacgaaggtgtacatgctttggaagccctggtagtcggtcttgctatccatgatgatgatctttcgtatgatcggtagcttcttttgcacgttgaggacctttcgcagccctttcttgctcacgaatacgacggtgggctggctgatgcccatgctgttcagcagctcgcgctcgttgtagatgtcgttagctggggccacagccacaccgatgaacagggcacccaacacgggcatgaagaactgcgagctattctcgctgcacaccacgatccgatggtttgtattcagcccatagcgcctcatagcttctgccagccgaacgctcatctcgaagtactcggcgtaggtaacgtccacctgaatatgtgcgtcggtaaaggcgatggcgccgggcaccagggcgtagcgcttcatggctttgtgcagctgctcgccggcggtcccgtcttcgagtgggtagaacggcgctgggcccttcttaatgtttttggcgtcttcgggtccgggattctcctcaacatccccgcatgtaagaagactccccctgccctcgccggagcccatgtcaaaggcatcctgcagttgggggagaggagggaagaaaaagaaagagtattagagagagagcagagaatgccaccctgaagagccctctccaataccaatggtcatccctaaaacatacgtatgaataacatgcagactaagcagggcggccgcacccagctttcttgtacaaagtgggaattcctagagctcgctgatcagcctcgacatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 7 - protein, 572 aa, synthetic construct
MGSGATNFSLLKQAGDVEENPGPEDAKNIKKGPAPFYPLEDGTAGEQLHKAMKRYALVPGAIAFTDAHIQVDVTYAEYFEMSVRLAEAMRRYGLNTNHRIVVCSENSSQFFMPVLGALFIGVAVAPANDIYNERELLNSMGISQPTVVFVSKKGLRKVLNVQKKLPIIRKIIIMDSKTDYQGFQSMYTFVTSHLPPSFNEYDFVPESFDRDKTIALIMNSSGSTGLPKGVALPHRTACVRFSHARDPIFGYQNIPDTAILSVVPFHHGFGMFTTLGYLICGFRVVLMYRFEEELFLRSLQDYKIQSALLVPTLFSCLAKSTLIDKYDLSSLREIASGGAPLSKEVGEAVAKRFRLPGIRQGYGLTETTNAVMITPEGDRKPGSVGKVVPFFEAKVVDLVTGKTLGVNQRGELCVRGPMIMSGYVNNPEATNALIDKDGWLHSGDIAYWDEDEHFFIVDRLKSLIKYKGYQVAPAELEGILLQHPYIFDAGVAGLPDDDAGELPAAVVVLEHGKTMTEKEIVDYVASQVTTAKKLRGGVVFVDEVPRGSTGKLDARKIREILTKAKKDGKIAV

SEQ ID NO: 8 - DNA, 4123 bp, synthetic construct
  source 1..4123: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttggcggccgccctctccaataccaatggtcatccctaaaacatacgtatgaataacatgcagactaagcaggagagtagtccagggtttccgagggtttccgtcgactcagctcgtctcgagggcgtactaactgggccctttcccttttttttcctcagatgggttctggagctacaaacttcagcctcctcaagcaagctggagacgtggaagagaacccaggaccttacccatacgatgttccagattacgcttctaccgccgtgctggaaaatcctggcctgggcagaaagctgagcgacttcggccaagagacaagctacatcgaggacaactgcaaccagaacggcgccatcagcctgatcttcagcctgaaagaagaagtgggcgccctggccaaggtgctgagactgttcgaagagaacgacgtgaacctgacacacatcgagagcagacccagcagactgaagaaggacgagtacgagttcttcacccacctggacaagcggagcctgcctgctctgaccaacatcatcaagatcctgcggcacgacatcggcgccacagtgcacgaactgagccgggacaagaaaaaggacaccgtgccatggttccccagaaccatccaagagctggacagattcgccaaccagatcctgagctatggcgccgagctggacgctgatcaccctggcttcaaggaccccgtgtaccgggccagaagaaagcagtttgccgatatcgcctacaactaccggcacggccagcctattcctcgggtcgagtacatggaagaggaaaagaaaacctggggcaccgtgttcaagaccctgaagtccctgtacaagacccacgcctgctacgagtacaaccacatcttcccactgctcgagaagtactgcggcttccacgaggacaatatccctcagctcgaggacgtgtcccagttcctgcaaacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagatccttcgcccagttcagccaagagatcggactggcttctctgggagcccctgacgagtacattgagaagctggccaccatctactggttcaccgtggagttcggcctgtgcaagcagggcgatagcatcaaggcttatggcgctggcctgctgtctagctttggcgagctgcagtactgtctgagcgagaagcctaagctgctgcccctggaactggaaaagaccgccatccagaactacaccgtgaccgagttccagcctctgtactacgtggccgagagcttcaacgacgccaaagaaaaagtgcggaacttcgccgccaccattcctcggcctttcagcgtccgctacgacccctacacacagcggatcgaggtgctggacaacacacagcagctgaaaattctggccgacagcatcaacagcgagatcggcatcctgtgcagcgccctgcagaaaatcaagtgatagcaagtttgtacaaaaaagcaggctgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggccgaaccggccgatatcaagcttcgaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccaattctggcccgcattaccggtcatcctgcagcctatcacttgattttttgcagggcggagcacagtatcccgatttcggaattgatgctgtcggccagtattttcagctgctgtgtattgtccagaacctcgattctctgggtgtacgggtcatagcggacggaaaaaggtctcgggattgtagcggcaaagttccgaaccttctccttagcgtcattgaaggattctgcgacgtagtacagaggctgaaactctgtcacggtgtaattctggatggctgtcttctcaagctccagtgggagcagttttggcttttcgctcaggcagtactgcagctctccaaaggagctcaggagtcccgctccgtacgccttgatagagtcgccttgtttacacaggccgaactccactgtgaaccagtaaatggtagccagtttttcaatgtactcatccggagctcccagggaggccaggccgatctcctgtgagaattgcgcaaaggacctatcgctaaacaacgggacgtggccgaggagttcatgacagatgtctggttctggggtgtacataggcttgctaccatgtcggatatactgcgtgcagtgaaaaacgcggaaggcaagcccgcccagaaagtctcggcttgacagcagacctgcgacaggcctaagtcgaaagcctgtacaggtttggagaaactgagacacatcctccagctgcgggatgttgtcttcgtggaaaccacagtacttctccagcaaagggaaaatatgattatattcgtaacaggcatgggttttatacaaagatttgagcgtcttgaatacagtcccccaggttttcttctcctcttccatgtactcgacccgggggatgggctgaccgtgcctataattataggcgatatcggcgaactgctttctccttgcgcggtagacagggtccttgaacccagggtgatctgcatcaagttctgctccgtaggacaaaatctgattggcaaacctgtccagttcttgaatagtgcgcggaaaccatggtacggtatcctttttcttatccctactgagttcgtgcacggtcgcgcctatatcatgtctgagaattttgatgatgtttgtcagggcggggaggctccgcttatcaagatgcgtgaaaaactcatattcatcttttttcaggcgactaggtcgagattcgatgtgtgtgaggtttacgtcgttctcttcgaaaagtcgcaggaccttggcgagtgccccgacttcttccttcaaagaaaagatcaatgagattgccccattctggttacaattatcttctatgtagctcgtttcctgtccaaaatcggagagtttccggcccagaccaggattctccaagacagctgtggaagcgtaatctggaacatcgtatgggtagggtccgggattctcctcaacatccccgcatgtaagaagactccccctgccctcgccggagcccatgtcaaaggcatcctgcagttgggggagaggagggaagaaaaagaaagagtattagagagagagcagagaatgccaccctgaagagccctctccaataccaatggtcatccctaaaacatacgtatgaataacatgcagactaagcagggcggccgcacccagctttcttgtacaaagtgggaattcctagagctcgctgatcagcctcgacatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 9 - protein, 483 aa, synthetic construct
MGSGATNFSLLKQAGDVEENPGPYPYDVPDYASTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 10 - DNA, 3265 bp, synthetic construct
  source 1..3265: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttggcggccgccctctccaataccaatggtcatccctaaaacatacgtatgaataacatgcagactaagcagggttgactgtgcctttcgatcgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccaccatgtacccatacgatgttccagattacgcttctaccgccgtgctggaaaatcctggcctgggcagaaagctgagcgacttcggccaagagacaagctacatcgaggacaactgcaaccagaacggcgccatcagcctgatcttcagcctgaaagaagaagtgggcgccctggccaaggtgctgagactgttcgaagagaacgacgtgaacctgacacacatcgagagcagacccagcagactgaagaaggacgagtacgagttcttcacccacctggacaagcggagcctgcctgctctgaccaacatcatcaagatcctgcggcacgacatcggcgccacagtgcacgaactgagccgggacaagaaaaaggacaccgtgccatggttccccagaaccatccaagagctggacagattcgccaaccagatcctgagctatggcgccgagctggacgctgatcaccctggcttcaaggaccccgtgtaccgggccagaagaaagcagtttgccgatatcgcctacaactaccggcacggccagcctattcctcgggtcgagtacatggaagaggaaaagaaaacctggggcaccgtgttcaagaccctgaagtccctgtacaagacccacgcctgctacgagtacaaccacatcttcccactgctcgagaagtactgcggcttccacgaggacaatatccctcagctcgaggacgtgtcccagttcctgcaaacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagatccttcgcccagttcagccaagagatcggactggcttctctgggagcccctgacgagtacattgagaagctggccaccatctactggttcaccgtggagttcggcctgtgcaagcagggcgatagcatcaaggcttatggcgctggcctgctgtctagctttggcgagctgcagtactgtctgagcgagaagcctaagctgctgcccctggaactggaaaagaccgccatccagaactacaccgtgaccgagttccagcctctgtactacgtggccgagagcttcaacgacgccaaagaaaaagtgcggaacttcgccgccaccattcctcggcctttcagcgtccgctacgacccctacacacagcggatcgaggtgctggacaacacacagcagctgaaaattctggccgacagcatcaacagcgagatcggcatcctgtgcagcgccctgcagaaaatcaagtgacaagtttgtacaaaaaagcaggctggtaccgatcacatatgcctttaattaaacactggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgcctctccaataccaatggtcatccctaaaacatacgtatgaataacatgcagactaagcagggcggccgcacccagctttcttgtacaaagtgggaattcctagagctcgctgatcagcctcgacatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 11 - protein, 461 aa, synthetic construct
MYPYDVPDYASTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 12 - DNA, 117 bp, synthetic construct
  source 1..117: mol_type = other DNA
ctgcgcgctcgctcgctcactgaggccgcccgggcgtcgggcgacctttggtcgcccggcctcagtgagcgagcgagcgcgcagagagggagtggccaactccatcactaggggttc

SEQ ID NO: 13 - DNA, 62 bp, synthetic construct
  source 1..62: mol_type = other DNA
cctctccaataccaatggtcatccctaaaacatacgtatgaataacatgcagactaagcagg

SEQ ID NO: 14 - DNA, 88 bp, synthetic construct
  source 1..88: mol_type = other DNA
agagtagtccagggtttccgagggtttccgtcgactcagctcgtctcgagggcgtactaactgggccctttcccttttttttcctcag

SEQ ID NO: 15 - DNA, 69 bp, synthetic construct
  source 1..69: mol_type = other DNA
atgggttctggagctacaaacttcagcctcctcaagcaagctggagacgtggaagagaacccaggacct

SEQ ID NO: 16 - DNA, 1647 bp, synthetic construct
  source 1..1647: mol_type = other DNA
gaagacgctaagaacatcaagaagggacccgctcccttttaccctctggaggatgggaccgccggagaacagctccataaggctatgaaacggtatgcattggttcccggggctatagccttcaccgatgctcatattcaagtagacgtaacctatgcagagtacttcgagatgtcagtccggctcgcagaagcaatgcggcgatacggtttgaacacaaatcaccgcatagtcgtttgttcagaaaactcctctcaattttttatgcccgtactcggagcactctttattggtgttgcagtggcccccgctaacgacatatacaacgagagggagttgctgaactcaatgggaattagccaaccaacagtggtattcgtttcaaagaagggactgcgcaaagttcttaacgtacagaagaaattgcccataatccgaaaaatcattattatggactccaaaaccgactatcaggggtttcagagcatgtataccttcgtcacctctcaccttcctccctcattcaacgagtatgattttgttcccgaaagttttgaccgcgacaaaaccattgcactgatcatgaatagtagtgggagcacagggcttccaaaaggagttgctttgcctcaccgaacagcttgcgtgcggttcagccatgctagagaccctatctttggctaccagaacattcccgatacagccatcctcagtgtagtcccattccaccatggtttcggaatgtttactacactcggatatttgatttgcgggtttcgcgtcgtgctcatgtatcgcttcgaagaggagttgtttctccgctccctgcaagattacaaaattcaatctgccttgctggtgcctactcttttcagttgtctggccaagtcaacccttatcgacaagtacgatctctcatcattgcgggaaatagcctctggcggcgcaccactgtcaaaagaagtaggtgaggcagtagcaaagcgctttagattgccaggcatcaggcagggttatggtctgacagaaacaaccaatgctgttatgattactccagagggcgaccggaagccaggttctgtgggtaaagtagtccctttcttcgaggcaaaggtagtcgacttggtgacagggaagaccctcggcgtgaaccagcggggggaattgtgtgtacggggtcctatgatcatgagtggctatgtcaacaatcctgaagcaaccaatgctctcatcgataaggatggctggttgcatagtggtgatattgcttactgggatgaggacgagcacttttttattgttgataggctcaaatccctgatcaaatacaagggataccaggtggccccagcagaacttgagggaatactcctccagcacccatacattttcgatgctggagttgccggtttgccagacgatgatgcaggagagcttccagctgcagtggttgttcttgaacacggaaaaactatgacagagaaggagatcgtagattatgtagctagccaggtgactacagccaagaagctcagaggaggagtcgtattcgtagatgaagtaccacggggatcaacaggcaaactggatgcacggaaaatcagagagattttgaccaaagccaagaaggatggaaaaattgccgtg

SEQ ID NO: 17 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 18 - DNA, 27 bp, synthetic construct
  source 1..27: mol_type = other DNA
ggccgaaccggccgatatcaagcttcg

SEQ ID NO: 19 - DNA, 192 bp, synthetic construct
  source 1..192: mol_type = other DNA
aaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 20 - DNA, 1647 bp, synthetic construct
  source 1..1647: mol_type = other DNA
cacggcgatctttccgtccttcttggccttagtgagaatctcgcggatcttgcgggcgtctaacttgccggtcgatcctctaggtacctcatccacaaacacaacaccaccgcgcagcttcttggcggttgtaacctggctggccacatagtccacgatctctttctcggtcatggttttaccgtgttccaacacgacgactgcggcgggcagctcgccggcatcgtcgtcgggcaggccggcgactccggcgtcgaagatgtaggggtgttgcagcaggatgccctccagttcggctggggctacctggtagcccttgtatttgatcagggacttcagccggtccacgatgaagaagtgctcgtcctcgtcccagtaggcgatgtcgccgctgtgcagccagccgtccttgtcgatgagagcgttcgtagcctcggggttgttaacgtagccgctcatgatcatggggccacggacgcacagctcaccgcgctggttcacacccagtgtcttacccgtgaccaagtctaccaccttagcctcgaagaagggcaccaccttgcctactgagccaggcttacggtccccctcgggggtgatcatgacggcgttggttgtttctgtcaggccatagccctggcggatgcctggtaggcggaagcgtttggccacggcctcacctacctccttgctgagcggcgccccgccgctggcgatctcgcgcaagctgcttaggtcgtacttgtcgatgagagtgctcttagcgaggcagctaaatagtgtgggcaccagcagggcagattgaatcttatagtcttgcaagctgcgcaagaatagctcctcctcgaagcggtacatgagcacgacccgaaagccgcagatcaagtagcccagcgtggtgaacatgccgaagccgtggtgaaatggcaccacgctgaggatagcggtgtcggggatgttctggtagccgaagatggggtcgcgggcatgactgaatcggacacaagcggtgcggtgcggtagggctacgcccttgggtaatcctgtactaccactactgttcatgatcagggcgatggttttgtcccggtcgaagctctcgggcacgaagtcgtactcgttgaagctgggtggcaaatgggaagtcacgaaggtgtacatgctttggaagccctggtagtcggtcttgctatccatgatgatgatctttcgtatgatcggtagcttcttttgcacgttgaggacctttcgcagccctttcttgctcacgaatacgacggtgggctggctgatgcccatgctgttcagcagctcgcgctcgttgtagatgtcgttagctggggccacagccacaccgatgaacagggcacccaacacgggcatgaagaactgcgagctattctcgctgcacaccacgatccgatggtttgtattcagcccatagcgcctcatagcttctgccagccgaacgctcatctcgaagtactcggcgtaggtaacgtccacctgaatatgtgcgtcggtaaaggcgatggcgccgggcaccagggcgtagcgcttcatggctttgtgcagctgctcgccggcggtcccgtcttcgagtgggtagaacggcgctgggcccttcttaatgtttttggcgtcttc

SEQ ID NO: 21 - DNA, 65 bp, synthetic construct
  source 1..65: mol_type = other DNA
gggtccgggattctcctcaacatccccgcatgtaagaagactccccctgccctcgccggagccca

SEQ ID NO: 22 - DNA, 79 bp, synthetic construct
  source 1..79: mol_type = other DNA
tgtcaaaggcatcctgcagttgggggagaggagggaagaaaaagaaagagtattagagagagagcagagaatgccaccc

SEQ ID NO: 23 - DNA, 62 bp, synthetic construct; identical to SEQ ID NO: 13
  source 1..62: mol_type = other DNA

SEQ ID NO: 24 - DNA, 142 bp, synthetic construct
  source 1..142: mol_type = other DNA
agagcgcccaatacgcaaaccgcctctccccgcgcgttggccgattcattaatgcagctggcacgacaggtttcccgactggaaagcgggcagtgagcgcaacgcaataggcgcccttaaggctagctagacctgcaggcag

SEQ ID NO: 25 - DNA, 1383 bp, synthetic construct
  source 1..1383: mol_type = other DNA
tacccatacgatgttccagattacgcttctaccgccgtgctggaaaatcctggcctgggcagaaagctgagcgacttcggccaagagacaagctacatcgaggacaactgcaaccagaacggcgccatcagcctgatcttcagcctgaaagaagaagtgggcgccctggccaaggtgctgagactgttcgaagagaacgacgtgaacctgacacacatcgagagcagacccagcagactgaagaaggacgagtacgagttcttcacccacctggacaagcggagcctgcctgctctgaccaacatcatcaagatcctgcggcacgacatcggcgccacagtgcacgaactgagccgggacaagaaaaaggacaccgtgccatggttccccagaaccatccaagagctggacagattcgccaaccagatcctgagctatggcgccgagctggacgctgatcaccctggcttcaaggaccccgtgtaccgggccagaagaaagcagtttgccgatatcgcctacaactaccggcacggccagcctattcctcgggtcgagtacatggaagaggaaaagaaaacctggggcaccgtgttcaagaccctgaagtccctgtacaagacccacgcctgctacgagtacaaccacatcttcccactgctcgagaagtactgcggcttccacgaggacaatatccctcagctcgaggacgtgtcccagttcctgcaaacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagatccttcgcccagttcagccaagagatcggactggcttctctgggagcccctgacgagtacattgagaagctggccaccatctactggttcaccgtggagttcggcctgtgcaagcagggcgatagcatcaaggcttatggcgctggcctgctgtctagctttggcgagctgcagtactgtctgagcgagaagcctaagctgctgcccctggaactggaaaagaccgccatccagaactacaccgtgaccgagttccagcctctgtactacgtggccgagagcttcaacgacgccaaagaaaaagtgcggaacttcgccgccaccattcctcggcctttcagcgtccgctacgacccctacacacagcggatcgaggtgctggacaacacacagcagctgaaaattctggccgacagcatcaacagcgagatcggcatcctgtgcagcgccctgcagaaaatcaagtga

SEQ ID NO: 26 - DNA, 1383 bp, synthetic construct
  source 1..1383: mol_type = other DNA
tcacttgattttttgcagggcggagcacagtatcccgatttcggaattgatgctgtcggccagtattttcagctgctgtgtattgtccagaacctcgattctctgggtgtacgggtcatagcggacggaaaaaggtctcgggattgtagcggcaaagttccgaaccttctccttagcgtcattgaaggattctgcgacgtagtacagaggctgaaactctgtcacggtgtaattctggatggctgtcttctcaagctccagtgggagcagttttggcttttcgctcaggcagtactgcagctctccaaaggagctcaggagtcccgctccgtacgccttgatagagtcgccttgtttacacaggccgaactccactgtgaaccagtaaatggtagccagtttttcaatgtactcatccggagctcccagggaggccaggccgatctcctgtgagaattgcgcaaaggacctatcgctaaacaacgggacgtggccgaggagttcatgacagatgtctggttctggggtgtacataggcttgctaccatgtcggatatactgcgtgcagtgaaaaacgcggaaggcaagcccgcccagaaagtctcggcttgacagcagacctgcgacaggcctaagtcgaaagcctgtacaggtttggagaaactgagacacatcctccagctgcgggatgttgtcttcgtggaaaccacagtacttctccagcaaagggaaaatatgattatattcgtaacaggcatgggttttatacaaagatttgagcgtcttgaatacagtcccccaggttttcttctcctcttccatgtactcgacccgggggatgggctgaccgtgcctataattataggcgatatcggcgaactgctttctccttgcgcggtagacagggtccttgaacccagggtgatctgcatcaagttctgctccgtaggacaaaatctgattggcaaacctgtccagttcttgaatagtgcgcggaaaccatggtacggtatcctttttcttatccctactgagttcgtgcacggtcgcgcctatatcatgtctgagaattttgatgatgtttgtcagggcggggaggctccgcttatcaagatgcgtgaaaaactcatattcatcttttttcaggcgactaggtcgagattcgatgtgtgtgaggtttacgtcgttctcttcgaaaagtcgcaggaccttggcgagtgccccgacttcttccttcaaagaaaagatcaatgagattgccccattctggttacaattatcttctatgtagctcgtttcctgtccaaaatcggagagtttccggcccagaccaggattctccaagacagctgtggaagcgtaatctggaacatcgtatgggta

SEQ ID NO: 27 - DNA, 984 bp, synthetic construct
  source 1..984: mol_type = other DNA
gggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccaccgatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaag

SEQ ID NO: 28 - protein, 1591 aa, synthetic construct
MAPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

SEQ ID NO: 29 - DNA, 4779 bp, synthetic construct
  source 1..4779: mol_type = other DNA
atggcaccaaagaagaaaagaaaagtggagggcatcaagtcaaacatcagcctgctgaaagacgaactgcggggacagattagtcacatcagtcacgagtacctgtcactgattgatctggccttcgacagcaagcagaatagactgtttgagatgaaagtgctggaactgctggtcaacgagtatggcttcaagggcagacatctgggcgggtctaggaaacctgacggcatcgtgtacagtaccacactggaagacaacttcggaatcattgtcgataccaaggcttattccgagggctactctctgccaattagtcaggcagatgagatggaaaggtacgtgcgcgaaaactcaaatagggacgaggaagtcaaccccaataagtggtgggagaatttcagcgaggaagtgaagaaatactacttcgtctttatctcaggcagcttcaaagggaagtttgaggaacagctgcggagactgtccatgactaccggggtgaacggatctgctgtcaacgtggtcaatctgctgctgggcgcagaaaagatcaggtccggggagatgacaattgaggaactggaacgcgccatgttcaacaattctgagtttatcctgaagtatggaggcgggggaagcgataagaaatactccatcggactggccattggcaccaattccgtgggctgggctgtcatcacagacgagtacaaggtgccaagcaagaagttcaaggtcctggggaacaccgatcgccacagtatcaagaaaaatctgattggagccctgctgttcgactcaggcgagactgctgaagcaacccgactgaagcggactgctaggcgccgatatacccggagaaaaaatcggatctgctacctgcaggaaattttcagcaacgagatggccaaggtggacgatagtttctttcaccgcctggaggaatcattcctggtggaggaagataagaaacacgagcggcatcccatctttggcaacattgtggacgaagtcgcttatcacgagaagtaccctactatctatcatctgaggaagaaactggtggactccaccgataaggcagacctgcgcctgatctatctggccctggctcacatgatcaagttccgggggcattttctgatcgagggagatctgaaccctgacaattctgatgtggacaagctgttcatccagctggtccagacatacaatcagctgtttgaggaaaacccaattaatgcctcaggcgtggacgcaaaggccatcctgagcgccagactgtccaaatctaggcgcctggaaaacctgatcgctcagctgccaggagagaagaaaaacggcctgtttgggaatctgattgcactgtccctgggcctgacacccaacttcaagtctaattttgatctggccgaggacgctaagctgcagctgtccaaagacacttatgacgatgacctggataacctgctggctcagatcggcgatcagtacgcagacctgttcctggccgctaagaatctgagtgacgccatcctgctgtcagatattctgcgcgtgaacacagagattactaaggccccactgagtgcttcaatgatcaaaagatatgacgagcaccatcaggatctgaccctgctgaaggctctggtgaggcagcagctgcccgagaaatacaaggaaatcttctttgatcagagcaagaatggatacgccggctatattgacggcggggcttcccaggaggagttctacaagttcatcaagcccattctggaaaagatggacggcaccgaggaactgctggtgaagctgaatcgggaggacctgctgagaaaacagaggacatttgataacggaagcatccctcaccagattcatctgggcgaactgcacgccatcctgcgacggcaggaggacttctacccatttctgaaggataaccgcgagaaaatcgaaaagatcctgaccttcagaatcccctactatgtggggcctctggcacggggaaatagtagatttgcctggatgacaagaaagtcagaggaaactatcaccccctggaacttcgaggaagtggtcgataaaggcgctagcgcacagtccttcattgaaaggatgacaaattttgacaagaacctgccaaatgagaaggtgctgcccaaacacagcctgctgtacgaatatttcacagtgtataacgagctgactaaagtgaagtacgtcaccgaagggatgcgcaagcccgcattcctgtccggagagcagaagaaagccatcgtggacctgctgtttaagacaaatcggaaagtgactgtcaaacagctgaaggaagactatttcaagaaaattgagtgtttcgattcagtggaaatcagcggcgtcgaggacaggtttaacgcctccctggggacctaccacgatctgctgaagatcatcaaggataaggacttcctggacaacgaggaaaatgaggacatcctggaggacattgtgctgacactgactctgtttgaggatcgcgaaatgatcgaggaacgactgaagacttatgcccatctgttcgatgacaaagtgatgaagcagctgaaaagaaggcgctacaccggatggggacgcctgagccgaaaactgatcaatgggattagagacaagcagagcggaaaaactatcctggactttctgaagtccgatggcttcgccaacaggaacttcatgcagctgattcacgatgactctctgaccttcaaggaggacatccagaaagcacaggtgtctggccagggggacagtctgcacgagcatatcgcaaacctggccggcagccccgccatcaagaaagggattctgcagaccgtgaaggtggtggacgaactggtcaaggtcatgggacgacacaaacctgagaacatcgtgattgagatggcccgcgaaaatcagacaactcagaagggccagaaaaacagtcgagaacggatgaagagaatcgaggaaggcatcaaggagctggggtcacagatcctgaaggagcatcctgtggaaaacactcagctgcagaatgagaaactgtatctgtactatctgcagaatggacgggatatgtacgtggaccaggagctggatattaacagactgagtgattatgacgtggatgccatcgtccctcagagcttcctgaaggatgactccattgacaacaaggtgctgaccaggtccgacaagaaccgcggcaaatcagataatgtgccaagcgaggaagtggtcaagaaaatgaagaactactggaggcagctgctgaatgccaagctgatcacacagcggaaatttgataacctgactaaggcagaaagaggaggcctgtctgagctggacaaggccggcttcatcaagcggcagctggtggagacaagacagatcactaagcacgtcgctcagattctggatagcagaatgaacacaaagtacgatgaaaacgacaagctgatcagggaggtgaaagtcattactctgaaatccaagctggtgtctgactttagaaaggatttccagttttataaagtcagggagatcaacaactaccaccatgctcatgacgcatacctgaacgcagtggtcgggaccgccctgattaagaaataccccaagctggagtccgagttcgtgtacggagactataaagtgtacgatgtccggaagatgatcgccaaatctgagcaggaaattggcaaggccaccgctaagtatttcttttacagtaacatcatgaatttctttaagaccgaaatcacactggcaaatggggagatcagaaaaaggcctctgattgagaccaacggggagacaggagaaatcgtgtgggacaagggaagggattttgctaccgtgcgcaaagtcctgtccatgccccaagtgaatattgtcaagaaaactgaagtgcagaccgggggattctctaaggagagtattctgcctaagcgaaactctgataaactgatcgcccggaagaaagactgggaccccaagaagtatggcgggttcgactctccaacagtggcttacagtgtcctggtggtcgcaaaggtggaaaaggggaagtccaagaaactgaagtctgtcaaagagctgctgggaatcactattatggaacgcagctccttcgagaagaatcctatcgattttctggaagccaagggctataaagaggtgaagaaagacctgatcattaagctgccaaaatactcactgtttgagctggaaaacggacgaaagcgaatgctggcaagcgccggagaactgcagaagggcaatgagctggccctgccctccaaatacgtgaacttcctgtatctggctagccactacgagaaactgaaggggtcccctgaggataacgaacagaagcagctgtttgtggagcagcacaaacattatctggacgagatcattgaacagatttcagagttcagcaagagagtgatcctggctgacgcaaatctggataaagtcctgagcgcatacaacaagcaccgagacaaaccaatccgggagcaggccgaaaatatcattcatctgttcaccctgacaaacctgggcgcccctgcagccttcaagtattttgacaccacaatcgatcggaagagatacacttctaccaaagaggtgctggatgctaccctgatccaccagagtattaccggcctgtatgagacacgcatcgacctgtcacagctgggaggcgatgggagccccaagaaaaagcggaaggtgtctagttaatga

SEQ ID NO: 30 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 31 - protein, 1053 aa, synthetic construct
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 32 - protein, 1368 aa, synthetic construct
  VARIANT 1
XDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 33 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 34 - protein, 199 aa, synthetic construct
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 35 - protein, 1591 aa, synthetic construct; identical to SEQ ID NO: 3

SEQ ID NO: 36 - DNA, 4779 bp, synthetic construct; identical to SEQ ID NO: 1
  source 1..4779: mol_type = other DNA

SEQ ID NO: 37 - protein, 1591 aa, synthetic construct; identical to SEQ ID NO: 28

SEQ ID NO: 38 - DNA, 4776 bp, synthetic construct
  source 1..4776: mol_type = other DNA
atggcaccaaagaagaaaagaaaagtggagggcatcaagtcaaacatcagcctgctgaaagacgaactgcggggacagattagtcacatcagtcacgagtacctgtcactgattgatctggccttcgacagcaagcagaatagactgtttgagatgaaagtgctggaactgctggtcaacgagtatggcttcaagggcagacatctgggcgggtctaggaaacctgacggcatcgtgtacagtaccacactggaagacaacttcggaatcattgtcgataccaaggcttattccgagggctactctctgccaattagtcaggcagatgagatggaaaggtacgtgcgcgaaaactcaaatagggacgaggaagtcaaccccaataagtggtgggagaatttcagcgaggaagtgaagaaatactacttcgtctttatctcaggcagcttcaaagggaagtttgaggaacagctgcggagactgtccatgactaccggggtgaacggatctgctgtcaacgtggtcaatctgctgctgggcgcagaaaagatcaggtccggggagatgacaattgaggaactggaacgcgccatgttcaacaattctgagtttatcctgaagtatggaggcgggggaagcgataagaaatactccatcggactggccattggcaccaattccgtgggctgggctgtcatcacagacgagtacaaggtgccaagcaagaagttcaaggtcctggggaacaccgatcgccacagtatcaagaaaaatctgattggagccctgctgttcgactcaggcgagactgctgaagcaacccgactgaagcggactgctaggcgccgatatacccggagaaaaaatcggatctgctacctgcaggaaattttcagcaacgagatggccaaggtggacgatagtttctttcaccgcctggaggaatcattcctggtggaggaagataagaaacacgagcggcatcccatctttggcaacattgtggacgaagtcgcttatcacgagaagtaccctactatctatcatctgaggaagaaactggtggactccaccgataaggcagacctgcgcctgatctatctggccctggctcacatgatcaagttccgggggcattttctgatcgagggagatctgaaccctgacaattctgatgtggacaagctgttcatccagctggtccagacatacaatcagctgtttgaggaaaacccaattaatgcctcaggcgtggacgcaaaggccatcctgagcgccagactgtccaaatctaggcgcctggaaaacctgatcgctcagctgccaggagagaagaaaaacggcctgtttgggaatctgattgcactgtccctgggcctgacacccaacttcaagtctaattttgatctggccgaggacgctaagctgcagctgtccaaagacacttatgacgatgacctggataacctgctggctcagatcggcgatcagtacgcagacctgttcctggccgctaagaatctgagtgacgccatcctgctgtcagatattctgcgcgtgaacacagagattactaaggccccactgagtgcttcaatgatcaaaagatatgacgagcaccatcaggatctgaccctgctgaaggctctggtgaggcagcagctgcccgagaaatacaaggaaatcttctttgatcagagcaagaatggatacgccggctatattgacggcggggcttcccaggaggagttctacaagttcatcaagcccattctggaaaagatggacggcaccgaggaactgctggtgaagctgaatcgggaggacctgctgagaaaacagaggacatttgataacggaagcatccctcaccagattcatctgggcgaactgcacgccatcctgcgacggcaggaggacttctacccatttctgaaggataaccgcgagaaaatcgaaaagatcctgaccttcagaatcccctactatgtggggcctctggcacggggaaatagtagatttgcctggatgacaagaaagtcagaggaaactatcaccccctggaacttcgaggaagtggtcgataaaggcgctagcgcacagtccttcattgaaaggatgacaaattttgacaagaacctgccaaatgagaaggtgctgcccaaacacagcctgctgtacgaatatttcacagtgtataacgagctgactaaagtgaagtacgtcaccgaagggatgcgcaagcccgcattcctgtccggagagcagaagaaagccatcgtggacctgctgtttaagacaaatcggaaagtgactgtcaaacagctgaaggaagactatttcaagaaaattgagtgtttcgattcagtggaaatcagcggcgtcgaggacaggtttaacgcctccctggggacctaccacgatctgctgaagatcatcaaggataaggacttcctggacaacgaggaaaatgaggacatcctggaggacattgtgctgacactgactctgtttgaggatcgcgaaatgatcgaggaacgactgaagacttatgcccatctgttcgatgacaaagtgatgaagcagctgaaaagaaggcgctacaccggatggggacgcctgagccgaaaactgatcaatgggattagagacaagcagagcggaaaaactatcctggactttctgaagtccgatggcttcgccaacaggaacttcatgcagctgattcacgatgactctctgaccttcaaggaggacatccagaaagcacaggtgtctggccagggggacagtctgcacgagcatatcgcaaacctggccggcagccccgccatcaagaaagggattctgcagaccgtgaaggtggtggacgaactggtcaaggtcatgggacgacacaaacctgagaacatcgtgattgagatggcccgcgaaaatcagacaactcagaagggccagaaaaacagtcgagaacggatgaagagaatcgaggaaggcatcaaggagctggggtcacagatcctgaaggagcatcctgtggaaaacactcagctgcagaatgagaaactgtatctgtactatctgcagaatggacgggatatgtacgtggaccaggagctggatattaacagactgagtgattatgacgtggatgccatcgtccctcagagcttcctgaaggatgactccattgacaacaaggtgctgaccaggtccgacaagaaccgcggcaaatcagataatgtgccaagcgaggaagtggtcaagaaaatgaagaactactggaggcagctgctgaatgccaagctgatcacacagcggaaatttgataacctgactaaggcagaaagaggaggcctgtctgagctggacaaggccggcttcatcaagcggcagctggtggagacaagacagatcactaagcacgtcgctcagattctggatagcagaatgaacacaaagtacgatgaaaacgacaagctgatcagggaggtgaaagtcattactctgaaatccaagctggtgtctgactttagaaaggatttccagttttataaagtcagggagatcaacaactaccaccatgctcatgacgcatacctgaacgcagtggtcgggaccgccctgattaagaaataccccaagctggagtccgagttcgtgtacggagactataaagtgtacgatgtccggaagatgatcgccaaatctgagcaggaaattggcaaggccaccgctaagtatttcttttacagtaacatcatgaatttctttaagaccgaaatcacactggcaaatggggagatcagaaaaaggcctctgattgagaccaacggggagacaggagaaatcgtgtgggacaagggaagggattttgctaccgtgcgcaaagtcctgtccatgccccaagtgaatattgtcaagaaaactgaagtgcagaccgggggattctctaaggagagtattctgcctaagcgaaactctgataaactgatcgcccggaagaaagactgggaccccaagaagtatggcgggttcgactctccaacagtggcttacagtgtcctggtggtcgcaaaggtggaaaaggggaagtccaagaaactgaagtctgtcaaagagctgctgggaatcactattatggaacgcagctccttcgagaagaatcctatcgattttctggaagccaagggctataaagaggtgaagaaagacctgatcattaagctgccaaaatactcactgtttgagctggaaaacggacgaaagcgaatgctggcaagcgccggagaactgcagaagggcaatgagctggccctgccctccaaatacgtgaacttcctgtatctggctagccactacgagaaactgaaggggtcccctgaggataacgaacagaagcagctgtttgtggagcagcacaaacattatctggacgagatcattgaacagatttcagagttcagcaagagagtgatcctggctgacgcaaatctggataaagtcctgagcgcatacaacaagcaccgagacaaaccaatccgggagcaggccgaaaatatcattcatctgttcaccctgacaaacctgggcgcccctgcagccttcaagtattttgacaccacaatcgatcggaagagatacacttctaccaaagaggtgctggatgctaccctgatccaccagagtattaccggcctgtatgagacacgcatcgacctgtcacagctgggaggcgatgggagccccaagaaaaagcggaaggtgtctagttaa

SEQ ID NO: 39 - protein, 1588 aa, synthetic construct
MPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 40 - DNA, 4767 bp, synthetic construct
  source 1..4767: mol_type = other DNA
atgcctaagaagaagcggaaggtggaaggcatcaaaagcaacatctccctcctgaaagacgaactccgggggcagattagccacattagtcacgaatacctctccctcatcgacctggctttcgatagcaagcagaacaggctctttgagatgaaagtgctggaactgctcgtcaatgagtacgggttcaagggtcgacacctcggcggatctaggaaaccagacggcatcgtgtatagtaccacactggaagacaactttgggatcattgtggataccaaggcatactctgagggttatagtctgcccatttcacaggccgacgagatggaacggtacgtgcgcgagaactcaaatagagatgaggaagtcaaccctaacaagtggtgggagaacttctctgaggaagtgaagaaatactacttcgtctttatcagcgggtccttcaagggtaaatttgaggaacagctcaggagactgagcatgactaccggcgtgaatggcagcgccgtcaacgtggtcaatctgctcctgggcgctgaaaagattcggagcggagagatgaccatcgaagagctggagagggcaatgtttaataatagcgagtttatcctgaaatacggtggcggtggatccgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctga

(19) United States
(12) Patent Application Publication
(10) Pub. No.: US 2026/0218237 A1
(43) Pub. Date: Jul. 30, 2026

(54) COMPOSITIONS AND METHODS FOR INTEGRATION OF VIRAL VECTORS

(71) Applicant: Poseida Therapeutics, Inc., San Diego, CA (US)

(72) Inventors: Oscar ALVAREZ, San Diego, CA (US); Blair B. MADISON, San Diego, CA (US)

(73) Assignee: POSEIDA THERAPEUTICS, INC., San Diego, CA (US)

(21) Appl. No.: 19/571,350

(22) Filed: Mar. 18, 2026

Related U.S. Application Data
(63) Continuation of application No. PCT/US2024/047216, filed on Sep. 18, 2024.
(60) Provisional application No. 63/583,701, filed on Sep. 19, 2023.

(51) Int. Cl. C12N 15/86; A61K 9/51; A61K 31/7105; A61K 38/44; A61K 38/46; A61K 48/00; A61P 3/00; C12N 9/02

(52) U.S. Cl. CPC C12N 15/86; A61K 9/5123; A61K 31/7105; A61K 38/44; A61K 38/465; A61K 48/0033; A61K 48/005; A61P 3/00; C12N 9/0071; C12Y 114/16001; C12N 2750/14143; C12N 2830/42; C12N 2830/50

(57) ABSTRACT

The present disclosure relates to compositions and methods for treating phenylketonuria (PKU). In particular, the present disclosure relates to AAV donor polynucleotide vectors and LNP compositions comprising a targeting gRNA pair and a nucleic acid encoding Cas-CLOVER fusion protein, and methods of using the compositions for treating PKU.

### CROSS-REFERENCE TO RELATED APPLICATIONS

This application is a continuation of International Patent Application No. PCT/US2024/047216, filed Sep. 18, 2024, which claims the benefit of U.S. Provisional Patent Application No. 63/583,701 filed Sep. 19, 2023. Each of the foregoing applications is herein incorporated by reference in its entirety.

### SEQUENCE LISTING

The instant application contains a Sequence Listing which has been submitted electronically in XML format and is herein incorporated by reference in its entirety. Said XML copy, created on Mar. 18, 2026, is named SL-30006-030222-US1.xml and is 99,238 bytes in size.

### FIELD

The present disclosure relates generally to novel AAV donor polynucleotides designed for site-specific integration into a target sequence of the genome of a cell, AAV donor polynucleotides comprising an expression cassette or promoterless, bidirectional expression cassettes for expressing a transgene, e.g., a human phenylalanine hydroxylase (PAH) gene, AAV donors vectors comprising said polynucleotides, compositions comprising the AAV donor vectors and lipid nanoparticles (“LNPs”) comprising a gRNA pair complementary to genomic target sequences and an mRNA encoding Cas-CLOVER, methods of preparing these polynucleotides and LNPs, and the use of these AAV donor vectors and LNPs for gene therapy applications, particularly for the treatment of phenylketonuria (PKU).

### BACKGROUND

There has been a long-felt but unmet need in the art for compositions and methods for delivering nucleic acids to cells and for genetically modifying cells in vivo, ex vivo and in vitro. Widely accepted gene delivery and genetic modification techniques, such as the use of viral vectors, including AAV vectors, can cause acute toxicity and harmful side-effects in patients. The present disclosure provides improved AAV donor polynucleotides designed to be site-specifically integrated into a target sequence of the genome of a cell, AAV donor polynucleotides comprising an expression cassette or promoterless, bidirectional expression cassettes for expressing a transgene, e.g., a human phenylalanine hydroxylase (PAH) gene, AAV donors vectors comprising said polynucleotides, compositions comprising the AAV donor vectors and lipid nanoparticles (“LNPs”) comprising a gRNA pair and an mRNA encoding Cas-CLOVER, to cells, including hepatocytes, in vivo with high efficiency and low toxicity. Thus, the compositions and methods of the present disclosure have applicability for gene therapy therapeutics for treating PKU.

The present disclosure provides for AAV donor polynucleotides comprising an expression cassette or a promoterless, bidirectional expression cassette comprising at least one transgene encoding a codon optimized and modified human phenylalanine hydroxylase (PAH) gene, AAV donor vector compositions comprising the polynucleotides, and methods for using the AAV donor vector compositions in combination with a LNP composition comprising a gRNA pair targeting a specific genomic sequence and at least one mRNA encoding Cas-CLOVER for the treatment of phenylketonuria (PKU). The compositions and methods are described in further detail herein.

### SUMMARY OF THE INVENTION

In one aspect, provided herein is an adeno-associated virus (AAV) donor polynucleotide comprising in the 5′ to 3′ direction: (a) a first AAV ITR sequence; (b) a first targeting sequence homologous to a first region of intron 3 of the albumin gene comprising the sequence of SEQ ID NO: 13; (c) a splice acceptor sequence; (d) a P2A sequence; (e) a sequence encoding a first codon optimized and modified PAH gene comprising the sequence of SEQ ID NO: 25; (f) a DNA spacer sequence; (g) an inverted polyA sequence; (h) an inverted sequence encoding a second codon optimized and modified PAH gene comprising the sequence of SEQ ID NO: 26; (i) an inverted P2A sequence; (j) an inverted splice acceptor sequence; (k) a second targeting sequence homologous to a second region of intron 3 of the albumin gene comprising the sequence of SEQ ID NO: 23; and (1) a second AAV ITR sequence.

In some embodiments, first AAV ITR sequence comprises the sequence of SEQ ID NO: 12; the splice acceptor sequence comprises the sequence of SEQ ID NO: 14; the P2A sequence comprises the sequence of SEQ ID NO: 15; the polyadenylation (poly(A)) sequence comprises the sequence of SEQ ID NO: 17; the DNA spacer sequence comprises the sequence of SEQ ID NO: 18; the inverted polyA sequence comprises the sequence of SEQ ID NO: 19; the inverted P2A sequence comprises the sequence of SEQ ID NO: 21; the inverted splice acceptor sequence comprises the sequence of SEQ ID NO: 22; and/or the second AAV ITR sequence comprises the sequence of SEQ ID NO: 24.

In another aspect, provided herein is an adeno-associated virus (AAV) donor polynucleotide comprising in the 5′ to 3′ direction: (a) a first AAV ITR sequence; (b) a first targeting sequence homologous to a first region of intron 3 of the albumin gene comprising the sequence of SEQ ID NO: 13; (c) a TTRe promoter sequence; (d) a splice acceptor sequence; (e) a P2A sequence; (f) a sequence encoding a codon optimized and modified PAH gene comprising the sequence of SEQ ID NO: 25; (g) a 3′UTR sequence; (h) a polyadenylation (poly(A)) sequence; (i) a DNA spacer sequence; (j) a second targeting sequence homologous to a second region of intron 3 of the albumin gene comprising the sequence of SEQ ID NO: 23; and (k) a second AAV ITR sequence.

In some embodiments, the first AAV ITR sequence comprises the sequence of SEQ ID NO: 12; the TTRe promoter sequence comprises the sequence of SEQ ID NO: 27; the splice acceptor sequence comprises the sequence of SEQ ID NO: 14; the P2A sequence comprises the sequence of SEQ ID NO: 15; the 3′ UTR sequence comprises the sequence of SEQ ID NO: 10; the polyadenylation (poly(A)) sequence comprises the sequence of SEQ ID NO: 17; the DNA spacer sequence comprises the sequence of SEQ ID NO: 18; the inverted polyA sequence comprises the sequence of SEQ ID NO: 19; and/or the second AAV ITR sequence comprises the sequence of SEQ ID NO: 24.

In some embodiments, the AAV donor polynucleotide comprises the nucleic acid sequence of SEQ ID NO: 8. In some embodiments, the AAV donor polynucleotide comprises the nucleic acid sequence of SEQ ID NO: 10.

In another aspect, provided herein is an AAV viral vector comprising an AAV donor polynucleotide described herein. In some embodiments, the AAV viral vector is an AAV1, AAV2, AAV3, AAV4, AAV5, AAV6, AAV7, AAV8, AAV9, AAV10 or AAV11 viral vector. In some embodiments, the AAV viral vector is an AAV8 or AAV9 viral vector.

In another aspect, provided herein is a composition comprising: (a) an AAV viral vector described herein; and (b) at least one LNP composition comprising at least one gRNA pair and at least one mRNA molecule encoding Cas-CLOVER. In some embodiments, the mRNA molecule comprises a 5′-CAP. In some embodiments, the at least one gRNA pair comprises a left gRNA comprising the sequence of SEQ ID NO: 4 and a right gRNA comprising the sequence of SEQ ID NO: 5. In some embodiments, the at least one LNP composition comprises about 54% of ssPalmO-Ph-P4C2 by moles, about 35% of cholesterol by moles, about 10% of DOPC by moles, and about 1% of DMG-PEG2000 by moles

In another aspect, provided herein is a pharmaceutical composition comprising an AAV viral vector or a composition described herein and a pharmaceutically acceptable carrier.

In another aspect, provided herein is a method of treating phenylketonuria (PKU) in a subject in need thereof comprising administering to the subject: (a) at least one therapeutically effective dose of an AAV donor polynucleotide, an AAV viral vector, a composition or a pharmaceutical composition described herein; and (b) at least one LNP composition comprising at least one gRNA pair and at least one mRNA molecule encoding Cas-CLOVER. In some embodiments, the at least one LNP composition comprises: about 54% of ssPalmO-Ph-P4C2 by moles, about 35% of cholesterol by moles, about 5% of DOPC by moles, about 5% of DSPC by moles, and about 1% of DMG-PEG2000 by moles. In some embodiments, the mRNA molecule comprises a 5′-CAP. In some embodiments, the gRNA pair comprises a left gRNA comprising the sequence of SEQ ID NO: 4 and a right gRNA comprising the sequence of SEQ ID NO: 5.

In another aspect, provided herein is a method of site-specifically integrating a transgene via a viral vector into the genome of at least one cell in a subject comprising administering to the subject (a) at least one therapeutically effective dose of an AAV donor polynucleotide, an AAV viral vector, a composition or a pharmaceutical composition described herein, and (b) at least one LNP composition comprising at least one gRNA pair and at least one mRNA molecule encoding Cas-CLOVER, wherein the viral vector DNA is integrated into the genome of at least one cell at the genomic cut site generated by Cas-CLOVER. In some embodiments, the at least one LNP composition comprises: about 54% of ssPalmO-Ph-P4C2 by moles, about 35% of cholesterol by moles, about 5% of DOPC by moles, about 5% of DSPC by moles, and about 1% of DMG-PEG2000 by moles. In some embodiments, the mRNA molecule further comprises a 5′-CAP. In some embodiments, the gRNA pair comprises a left gRNA comprising the sequence of SEQ ID NO: 4 and a right gRNA comprising the sequence of SEQ ID NO: 5.

### BRIEF DESCRIPTION OF THE DRAWINGS

The above and further features will be more clearly appreciated from the following detailed description when taken in conjunction with the accompanying drawings.

FIG. 1A is a schematic diagram of an exemplary AAV donor polynucleotide comprising a promoterless, bidirectional luciferase cassette. FIG. 1B is a schematic diagram of an exemplary AAV donor polynucleotide comprising a promoterless, bidirectional human phenylalanine hydroxylase (PAH) cassette comprising a codon optimized and modified human PAH gene of the present disclosure. FIG. 1C is a schematic diagram of an exemplary AAV donor polynucleotide comprising a human TTRe-PAH expression cassette comprising a TTRe promoter sequence controlling expression of the human PAH gene.

FIG. 2A shows two graphs: a graph showing whole body luminescence imaging (BLI) and a graph showing percent albumin gene editing in wild type treated mice administered a low dose, medium dose or high dose of an AAV donor DNA comprising a promoterless, bidirectional luciferase expression cassette and an LNP composition comprising an albumin targeting gRNA pair and an mRNA encoding Cas-CLOVER. FIG. 2B shows two graphs: a graph showing percent integrations per haploid genome and a graph showing luciferase mRNA expression in wild type treated mice administered a low dose, medium dose or high dose of an AAV donor DNA comprising a promoterless, bidirectional luciferase expression cassette and an LNP composition comprising an albumin targeting gRNA pair and an mRNA encoding Cas-CLOVER.

FIG. 3A shows two graphs: a graph showing percent albumin gene editing and a graph showing relative human PAH mRNA expression in wild type treated mice administered a low dose, medium dose or high dose of an AAV donor DNA comprising a promoterless, bidirectional PAH expression cassette or an AAV donor DNA comprising a TTRe-PAH promoter expression cassette and an LNP composition comprising an albumin targeting gRNA pair and an mRNA encoding Cas-CLOVER. FIG. 3B shows two graphs of percent of total integrations per haploid genome in wild type treated mice administered a low dose, medium dose or high dose of an AAV donor DNA comprising a promoterless, bidirectional PAH expression cassette or an AAV donor DNA comprising a TTRe-PAH promoter expression cassette and an LNP composition comprising an albumin targeting gRNA pair and an mRNA encoding Cas-CLOVER.

FIG. 4A shows a graph of relative human PAH mRNA expression in BTBR PAH^{enu} treated mice administered a medium dose or high dose of an AAV donor DNA comprising a promoterless, bidirectional PAH expression cassette and an LNP composition comprising an albumin targeting gRNA pair and an mRNA encoding Cas-CLOVER. FIG. 4B shows a graph of percent of total integrations per haploid genome in BTBR PAH^{enu} treated mice administered a low dose, medium dose or high dose of an AAV donor DNA comprising a promoterless, bidirectional PAH expression cassette or an AAV donor DNA comprising a TTRe-PAH promoter expression cassette and an LNP composition comprising an albumin targeting gRNA pair and an mRNA encoding Cas-CLOVER.

FIG. 5A shows a graph of serum Phe levels (μM) in male BTBR PAH^{enu} mice treated with vehicle, an AAV donor DNA comprising a promoterless, bidirectional PAH expression cassette and an LNP composition comprising an albumin Intron 3 targeting gRNA pair and an mRNA encoding Cas-CLOVER at Days 0, 7, 14 and 28 post administration. FIG. 5B shows a graph of serum Phe levels (μM) in male BTBR PAH^{enu} mice treated with vehicle, an AAV donor DNA comprising a TTRe-PAH promoter expression cassette and an LNP composition comprising an albumin Intron 3 targeting gRNA pair and an mRNA encoding Cas-CLOVER at Days 0, 7, 14 and 28 post administration.

### DETAILED DESCRIPTION

### AAV Donor Polynucleotides

In certain aspects of the present disclosure, provided is an AAV donor polynucleotide comprising a promoterless, bidirectional expression cassette for expressing a transgene upon integration into a target sequence in a gene of a cell. In some embodiments, the AAV donor polynucleotide comprises a first and a second AAV ITR located at the 5′ and 3′-termini. In some embodiments, the AAV donor polynucleotide comprises a first target sequence and second target sequence that are homologous to a gene in the genome of a cell and positioned adjacent to the first and second ITRs, respectively. The first and second target sequences are designed to flank a portion of the target gene that is upstream and downstream of the site in the genome to be cut by the Cas-CLOVER enzyme to allow for site-specific integration of the AAV donor polynucleotide into the cell genome. In addition, the first and second targeting sequences also are complementary to the first and second gRNA, respectively, of the gRNA pair to allow for Cas-CLOVER to cut the AAV donor polynucleotide linearizing the polynucleotide and cleaving away the first and second AAV ITRs prior to integration into the cell genome.

In some embodiments, the first and second targeting sequence each comprise a nucleic acid sequence homologous to sequences of an intron of a gene. In some embodiments, the intron is intron 3 of the albumin gene. In some embodiments, the AAV donor polynucleotide targeting an intron comprises a splice acceptor sequence and a P2A ribosome skip sequence upstream of the coding sequence of the transgene, which is followed by a poly(A) polyadenylation sequence (poly(A)). The two expression cassettes are cloned into the AAV donor polynucleotide in opposite orientations (i.e., inverted) thereby allowing expression of the transgene irrespective as to whether the AAV donor polynucleotide is integrated at the Cas-CLOVER cut site in the forward or reverse orientation, with expression of the transgene being controlled by the regulatory elements and promoter of the targeted Albumin gene sequence.

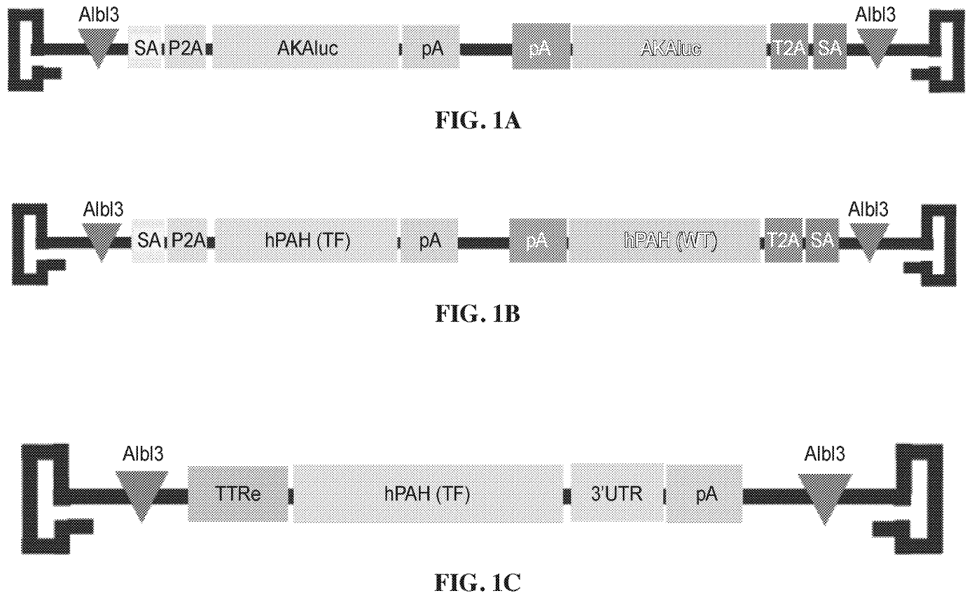

In certain aspects, the transgene is a reporter gene. In some embodiments, the transgene encodes luciferase. In certain aspects, the transgene is a human PAH gene. In one embodiment, the nucleotide sequence of the PAH gene is codon optimized to improve expression of the encoded human PAH enzyme. In other embodiments, the nucleotide sequence of the human PAH gene is modified to remove any undesired restriction enzyme recognition sites; and any putative cryptic splice sites for cloning the modified sequences into AAV donor vectors. In certain embodiments, the nucleotide sequence of the human PAH gene is codon optimized and further modified as described above. In some embodiments, the human PAH gene further comprises a hemagglutinin tag. Two exemplary AAV donor polynucleotides comprising a codon optimized and modified PAH gene are shown schematically in FIGS. 1B and 1C, respectively.

In certain aspects of the present disclosure, the AAV donor polynucleotide comprises a bidirectional luciferase reporter cassette. The AAV donor polynucleotide comprises in the 5′ to 3′ direction: a) a first AAV ITR sequence; b) a first target sequence homologous to a first portion of intron 3 of the albumin gene; c) a splice acceptor sequence; d) a P2A sequence; e) a luciferase coding sequence; f) a poly(A) sequence; g) a DNA spacer sequence; h) an inverted poly(A) sequence; i) an inverted luciferase coding sequence; j) an inverted P2A sequence; k) an inverted splice acceptor sequence; 1) a second target sequence homologous to a second portion of intron 3 of the albumin gene; and m) a second AAV ITR sequence. In one embodiment, the luciferase nucleic acid coding sequence and the inverted nucleic acid coding sequence differ in the primary nucleic acid sequences (with synonymous codons) to minimize the potential of internal homologous recombination of the two coding sequences.

In one embodiment, the first AAV ITR sequence comprises the nucleic acid of SEQ ID NO: 12. In one embodiment, the first target sequence homologous to an upstream portion of intron 3 of the albumin gene comprises the nucleic acid of SEQ ID NO: 13. In one embodiment, the splice acceptor sequence comprises the nucleic acid of SEQ ID NO: 14. In one embodiment, the P2A sequence comprises the nucleic acid of SEQ ID NO: 15. In one embodiment, the luciferase coding sequence comprises the nucleic acid of SEQ ID NO: 16. In one embodiment, the poly(A) sequence comprises the nucleic acid of SEQ ID NO: 17. In one embodiment, the DNA spacer sequence comprises the nucleic acid of SEQ ID NO: 18; In one embodiment, the inverted poly(A) sequence comprises the nucleic acid of SEQ ID NO: 19; In one embodiment, the inverted luciferase coding sequence comprises the nucleic acid of SEQ ID NO: 20. In one embodiment, the inverted P2A sequence comprises the nucleic acid of SEQ ID NO: 21. In one embodiment, the inverted splice acceptor sequence comprises the nucleic acid of SEQ ID NO: 22. In one embodiment, the second target sequence homologous to a second portion of intron 3 of the albumin gene comprises the nucleic acid of SEQ ID NO: 23; and second AAV ITR sequence gene comprises the nucleic acid of SEQ ID NO: 24.

In certain aspects of the present disclosure, the AAV donor polynucleotide comprises a bidirectional luciferase reporter cassette. In one embodiment, the transgene PAH nucleotide sequence further comprises a hemagglutinin (HA) tag. In one embodiment, the transgene PAH nucleotide sequence is codon optimized. In one embodiment, the transgene PAH nucleotide sequence is modified. In one embodiment, the transgene PAH nucleotide sequence comprises the hemagglutinin tag, codon optimized and further modified as disclosed herein. In one embodiment, the nucleic acid sequence encoding the human PAH gene sequence comprises the nucleic acid of SEQ ID NO: 25. In one embodiment, the PAH gene nucleic acid coding sequence and the inverted PAH nucleic acid coding sequence differ in the primary nucleic acid sequences (with synonymous codons) to minimize the potential of internal homologous recombination between the two coding sequences.

In certain aspects of the present disclosure, the AAV donor polynucleotide comprises a bidirectional PAH expression cassette. The AAV donor polynucleotide comprises in the 5′ to 3′ direction: a) a first AAV ITR sequence; b) a first target sequence homologous to a first portion of intron 3 of the albumin gene; c) a splice acceptor sequence; d) a P2A sequence; e) a PAH coding sequence; f) a poly(A) sequence; g) a DNA spacer sequence; h) an inverted poly(A) sequence; i) an inverted PAH coding sequence; j) an inverted P2A sequence; k) an inverted splice acceptor sequence; 1) a second target sequence homologous to a second portion of intron 3 of the albumin gene; and m) a second AAV ITR sequence. In one embodiment, the luciferase nucleic acid coding sequence and the inverted nucleic acid coding sequence differ in the primary nucleic acid sequences (with synonymous codons) to minimize the potential of internal homologous recombination of the two coding sequences.

In one embodiment, the first AAV ITR sequence comprises the nucleic acid of SEQ ID NO: 12. In one embodiment, the first target sequence homologous to a first portion of intron 3 of the albumin gene comprises the nucleic acid of SEQ ID NO: 13. In one embodiment, the splice acceptor sequence comprises the nucleic acid of SEQ ID NO: 14. In one embodiment, the P2A sequence comprises the nucleic acid of SEQ ID NO: 15. In one embodiment, the PAH coding sequence comprises the nucleic acid of SEQ ID NO: 25. In one embodiment, the poly(A) sequence comprises the nucleic acid of SEQ ID NO: 17. In one embodiment, the DNA spacer sequence comprises the nucleic acid of SEQ ID NO: 18; In one embodiment, the inverted poly(A) sequence comprises the nucleic acid of SEQ ID NO: 19; In one embodiment, the inverted PAH coding sequence comprises the nucleic acid of SEQ ID NO: 26. In one embodiment, the inverted P2A sequence comprises the nucleic acid of SEQ ID NO: 21. In one embodiment, the inverted splice acceptor sequence comprises the nucleic acid of SEQ ID NO: 22. In one embodiment, the second target sequence homologous to a second portion of intron 3 of the albumin gene comprises the nucleic acid of SEQ ID NO: 23; and second AAV ITR sequence gene comprises the nucleic acid of SEQ ID NO: 24.

In certain aspects of the present disclosure, the AAV donor polynucleotide comprises a TTRe-PAH expression cassette. In one embodiment, expression of the PAH gene is controlled by the heterologous TTRe promoter operably associated with the PAH coding sequence.

In some embodiments, the AAV donor polynucleotide comprises in the 5′ to 3′ direction: a) a first AAV ITR sequence; b) a first target sequence homologous to a first portion of intron 3 of the albumin gene; c) a TTRe promoter sequence; d) a PAH coding sequence; d) a 3′UTR sequence; f) a poly(A) sequence; g) a second target sequence homologous to a second portion of Intron 3 of the albumin gene; and h) a second AAV ITR sequence.

In one embodiment, the first AAV ITR sequence comprises the nucleic acid of SEQ ID NO: 12. In one embodiment, the first target sequence homologous to an upstream portion of intron 3 of the albumin gene comprises the nucleic acid of SEQ ID NO: 13. In one embodiment, the TTRe promoter sequence comprises the nucleic acid of SEQ ID NO: 27. In one embodiment, the PAH coding sequence comprises the nucleic acid of SEQ ID NO: 25. In one embodiment, the 3′-UTR sequence comprises the nucleic acid of SEQ ID NO: 27. In one embodiment, the poly(A) sequence comprises the nucleic acid of SEQ ID NO: 17. In one embodiment, the first target sequence homologous to a second portion of intron 3 of the albumin gene comprises the nucleic acid of SEQ ID NO: 23; and second AAV ITR sequence gene comprises the nucleic acid of SEQ ID NO: 24.

### Lipid Nanoparticles

The present disclosure provides a composition comprising at least one lipid nanoparticle comprising at least one cationic lipid and at least one nucleic acid molecule. In some aspects, a lipid nanoparticle can further comprise at least one structural lipid. In some aspects, a lipid nanoparticle can further comprise at least one phospholipid. In some aspects, a lipid nanoparticle can further comprise at least one PEGylated lipid.

Accordingly, the present disclosure provides compositions comprising at least one lipid nanoparticle, wherein the at least one lipid nanoparticle comprises at least one cationic lipid, at least one nucleic acid molecule, at least one structural lipid, at least one phospholipid and at least one PEGylated lipid.

### Bioreducible Ionizable Cationic Lipids

In some aspects, a cationic lipid can be a bioreducible ionizable cationic lipid.

Accordingly, the present disclosure provides compositions comprising at least one lipid nanoparticle, wherein the at least one lipid nanoparticle comprises at least one bioreducible ionizable cationic lipid.

As used herein, the term “bioreducible ionizable cationic lipid” is used in its broadest sense to refer to a cationic lipid comprising: at least one tertiary amine, at least one disulfide group, at least one group comprising a bond that is susceptible to cleavage by thioesterification, and further comprising at least two saturated or unsaturated hydrocarbon chains. Exemplary bioreducible ionizable cationic lipids include, but are not limited to, those described in Akita et al., (2020) Biol. Phar. Bull. 43:1617-1625, the contents of which are incorporated herein by reference in their entirety.

Additional exemplary bioreducible ionizable cationic lipids and methods of preparing such lipids useful in the methods of the present disclosure include those disclosed in International Patent Application No. PCT/JP2016/052690, published as WO/2016/121942 and International Patent Application No. PCT/JP2019/012302, published as WO/2019/188867, the contents of each of which are incorporated herein by reference in their entirety for examples of lipids that may be used in the compositions disclosed herein and for methods that may be used to make the compositions disclosed herein.

Accordingly, the present disclosure provides compositions comprising at least one lipid nanoparticle, wherein the at least one lipid nanoparticle comprises any one of the bioreducible ionizable cationic lipids set forth in WO/2016/121942 and WO/2019/188867.

Accordingly, the present disclosure provides compositions comprising at least one lipid nanoparticle, wherein the at least one lipid nanoparticle comprises at least one bioreducible ionizable cationic lipid, at least one nucleic acid molecule, at least one structural lipid, at least one phospholipid and at least one PEGylated lipid.

In some aspects, the bioreducible ionizable cationic lipid can be ssPalmO-Ph-P4C2, having the following structure:

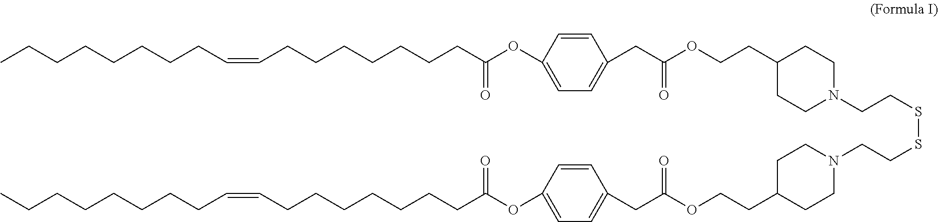 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

See Akita et al., (2020) Biol. Phar. Bull. 43:1617-1625, the contents of which are incorporated by reference in their entirety for examples of lipids that may be used in the compositions described herein.

Accordingly, the present disclosure provides compositions comprising at least one lipid nanoparticle comprising at least one bioreducible ionizable cationic lipid, wherein the at least one bioreducible ionizable cationic lipid comprises ssPalmO-Ph-P4C2.

As would be appreciated by the skilled artisan, ssPalmO-Ph-P4C2 can also be referred to as Coatsome® SS-OP, ssPalmO-Phe-P4C2, ssPalmO-Phenyl-P4C2, ssPalmO-Phe and ssPalmO-Ph. Accordingly, ssPalmO-Ph-P4C2, Coatsome® SS-OP, ssPalmO-Phe-P4C2, ssPalmO-Phenyl-P4C2, ssPalmO-Phe and ssPalmO-Ph are used interchangeably herein to refer to the bioreducible ionizable cationic lipid with the chemical structure set forth in Formula I.

As described herein, the LNP compositions of the present disclosure that comprise at least one bioreducible ionizable cationic lipid advantageously exhibit significantly reduced toxicity in animals as compared to LNP compositions comprising non-bioreducible ionizable cationic lipids. In particular, administration the LNP compositions of the present disclosure surprisingly does not result in any body weight loss. In some aspects, certain LNP compositions of the present disclosure are sufficiently non-toxic that animals administered the LNPs actually gain body weight, even when administered amounts of LNPs that exceed the lethal dose of LNP compositions comprising non-bioreducible ionizable cationic lipids.

### LNP Components

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least one bioreducible ionizable cationic lipid by moles.

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one bioreducible ionizable cationic lipid by moles.

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least one structural lipid by moles.

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one structural lipid by moles.

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5%, or about 60%, or about 62.5%, or about 65%, or about 67.5%, or about 70% of at least one phospholipid by moles.

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5%, or at least about 60%, or at least about 62.5%, or at least about 65%, or at least about 67.5%, or at least about 70% of at least one phospholipid by moles.

In some aspects, an LNP of the present disclosure can comprise about 0.25%, or about 0.5%, or about 0.75%, or about 1.0%, or about 1.25%, or about 1.5%, or about 1.75%, or about 2.0%, or at least about or about 2.5%, or about 5% of at least one PEGylated lipid by moles.

In some aspects, an LNP of the present disclosure can comprise at least about 0.25%, or at least about 0.5%, or at least about 0.75%, or at least about 1.0%, or at least about 1.25%, or at least about 1.5%, or at least about 1.75%, or at least about 2.0%, or at least about 2.5%, or at least about 5% of at least one PEGylated lipid by moles.

### Structural Lipids

In some aspects, a structural lipid can be a steroid. In some aspects, a structural lipid can be a sterol. In some aspects, a structural lipid can comprise cholesterol. In some aspects, a structural lipid can comprise ergosterol. In some aspects, a structural lipid can be a phytosterol.

### Phospholipid

As used herein, the term “phospholipid” is used in its broadest sense to refer to any amphiphilic molecule that comprises a polar (hydrophilic) headgroup comprising phosphate and two hydrophobic fatty acid chains.

In some aspects of the lipid nanoparticles of the present disclosure, a phospholipid can comprise dioleoylphosphatidylethanolamine (DOPE).

In some aspects of the lipid nanoparticles of the present disclosure, a phospholipid can comprise DOPC (1,2-Dioleoyl-sn-glycero-3-phosphocholine).

In some aspects of the lipid nanoparticles of the present disclosure, a phospholipid can comprise DSPC (1,2-Distearoyl-sn-glycero-3-phosphocholine).

In some aspects, a phospholipid can comprise DDPC (1,2-Didecanoyl-sn-glycero-3-phosphocholine), DEPA-NA (1,2-Dierucoyl-sn-glycero-3-phosphate (Sodium Salt)), DEPC (1,2-Dierucoyl-sn-glycero-3-phosphocholine), DEPE (1,2-Dierucoyl-sn-glycero-3-phosphoethanolamine), DEPG-NA (1,2-Dierucoyl-sn-glycero-3 [Phospho-rac-(1-glycerol) (Sodium Salt)), DLOPC (1,2-Dilinoleoyl-sn-glycero-3-phosphocholine), DLPA-NA (1,2-Dilauroyl-sn-glycero-3-phosphate (Sodium Salt)), DLPC (1,2-Dilauroyl-sn-glycero-3-phosphocholine), DLPE (1,2-Dilauroyl-sn-glycero-3-phosphoethanolamine), DLPG-NA (1,2-Dilauroyl-sn-glycero-3 [Phospho-rac-(1-glycerol) (Sodium Salt)), DLPG-NH4 (1,2-Dilauroyl-sn-glycero-3 [Phospho-rac-(1-glycerol) (Ammonium Salt)), DLPS-NA (1,2-Dilauroyl-sn-glycero-3-phosphoserine (Sodium Salt)), DMPA-NA (1,2-Dimyristoyl-sn-glycero-3-phosphate (Sodium Salt)), DMPC (1,2-Dimyristoyl-sn-glycero-3-phosphocholine), DMPE (1,2-Dimyristoyl-sn-glycero-3-phosphoethanolamine), DMPG-NA (1,2-Dimyristoyl-sn-glycero-3 [Phospho-rac-(1-glycerol) (Sodium Salt)), DMPG-NH4 (1,2-Dimyristoyl-sn-glycero-3 [Phospho-rac-(1-glycerol) (Ammonium Salt)), DMPG-NH4/NA (1,2-Dimyristoyl-sn-glycero-3 [Phospho-rac-(1-glycerol) (Sodium/Ammonium Salt)), DMPS-NA (1,2-Dimyristoyl-sn-glycero-3-phosphoserine (Sodium Salt)), DOPA-NA (1,2-Dioleoyl-sn-glycero-3-phosphate (Sodium Salt)), DOPC (1,2-Dioleoyl-sn-glycero-3-phosphocholine), DOPE (1,2-Dioleoyl-sn-glycero-3-phosphoethanolamine), DOPG-NA (1,2-Dioleoyl-sn-glycero-3 [Phospho-rac-(1-glycerol) (Sodium Salt)), DOPS-NA (1,2-Dioleoyl-sn-glycero-3-phosphoserine (Sodium Salt)), DPPA-NA (1,2-Dipalmitoyl-sn-glycero-3-phosphate (Sodium Salt)), DPPC (1,2-Dipalmitoyl-sn-glycero-3-phosphocholine), DPPE (1,2-Dipalmitoyl-sn-glycero-3-phosphoethanolamine), DPPG-NA (1,2-Dipalmitoyl-sn-glycero-3 [Phospho-rac-(1-glycerol) (Sodium Salt)), DPPG-NH4 (1,2-Dipalmitoyl-sn-glycero-3 [Phospho-rac-(1-glycerol) (Ammonium Salt)), DPPS-NA (1,2-Dipalmitoyl-sn-glycero-3-phosphoserine (Sodium Salt)), DSPA-NA (1,2-Distearoyl-sn-glycero-3-phosphate (Sodium Salt)), DSPC (1,2-Distearoyl-sn-glycero-3-phosphocholine), DSPE (1,2-Distearoyl-sn-glycero-3-phosphoethanolamine), DSPG-NA (1,2-Distearoyl-sn-glycero-3 [Phospho-rac-(1-glycerol) (Sodium Salt)), DSPG-NH4 (1,2-Distearoyl-sn-glycero-3 [Phospho-rac-(1-glycerol) (Ammonium Salt)), DSPS-NA (1,2-Distearoyl-sn-glycero-3-phosphoserine (Sodium Salt)), EPC (Egg-PC), HEPC (Hydrogenated Egg PC), HSPC (Hydrogenated Soy PC), LYSOPC MYRISTIC (1-Myristoyl-sn-glycero-3-phosphocholine), LYSOPC PALMITIC (1-Palmitoyl-sn-glycero-3-phosphocholine), LYSOPC STEARIC (1-Stearoyl-sn-glycero-3-phosphocholine), Milk Sphingomyelin (MPPC; 1-Myristoyl-2-palmitoyl-sn-glycero 3-phosphocholine), MSPC (1-Myristoyl-2-stearoyl-sn-glycero-3-phosphocholine), PMPC (1-Palmitoyl-2-myristoyl-sn-glycero-3-phosphocholine), POPC (1-Palmitoyl-2-oleoyl-sn-glycero-3-phosphocholine), POPE (1-Palmitoyl-2-oleoyl-sn-glycero-3-phosphoethanolamine), POPG-NA (1-Palmitoyl-2-oleoyl-sn-glycero-3 [Phospho-rac-(1-glycerol)] (Sodium Salt)), PSPC (1-Palmitoyl-2-stearoyl-sn-glycero-3-phosphocholine), SMPC (1-Stearoyl-2-myristoyl-sn-glycero-3-phosphocholine), SOPC (1-Stearoyl-2-oleoyl-sn-glycero-3-phosphocholine), SPPC (1-Stearoyl-2-palmitoyl-sn-glycero-3-phosphocholine), or any combination thereof.

### PEGylated Lipid

As used herein, the term “PEGylated lipid” is used to refer to any lipid that is modified (e.g., covalently linked to) at least one polyethylene glycol molecule. In some aspects, a PEGylated lipid can comprise 1,2-dimyristoyl-rac-glycero-3-methoxypoly ethylene glycol-2000, hereafter referred to as DMG-PEG2000.

### Nucleic Acids

In some aspects, a lipid nanoparticle can comprise at least one nucleic acid molecule. In some aspects, a lipid nanoparticle can comprise a plurality of nucleic acid molecules. In some aspects, the at least one nucleic acid molecule or the plurality of nucleic acid molecules can be formulated in a lipid nanoparticle.

In some aspects, a nucleic acid molecule can be a synthetic nucleic acid molecule. In some aspects, a nucleic acid molecule can be a non-naturally occurring nucleic acid molecule. In some aspects, a non-naturally occurring nucleic acid molecule can comprise at least one non-naturally occurring nucleotide. The at least one non-naturally occurring nucleotide can be any non-naturally occurring nucleotide known in the art. In some aspects, a nucleic acid molecule can be a modified nucleic acid molecule. In some aspects, a modified nucleic acid molecule can comprise at least one modified nucleotide. The at least one modified nucleotide can be any modified nucleic acid known in the art. In some embodiments, the modified nucleic acid is a gRNA or gRNA pair. In some embodiments, the modified nucleic acid is an mRNA encoding Cas-CLOVER.

In some aspects, a lipid nanoparticle can comprise lipid and nucleic acid at a specified ratio (weight/weight).

In some aspects, a lipid nanoparticle comprising at least one nucleic acid molecule can comprise lipid and nucleic acid at a ratio of about 5:1 to about 15:1, or about 10:1 to about 20:1, or about 15:1 to about 25:1, or about 20:1 to about 30:1, or about 25:1 to about 35:1 or about 30:1 to about 40:1, or about 35:1 to about 45:1, or about 40:1 to about 50:1, or about 45:1 to about 55:1, or about 50:1 to about 60:1, or about 55:1 to about 65:1, or about 60:1 to about 70:1, or about 65:1 to about 75:1, or about 70:1 to about 80:1, or about 75:1 to about 85:1, or about 80:1 to about 90:1, or about 85:1 to about 95:1, or about 90:1 to about 100:1, or about 95:1 to about 105:1, or about 100:1 to about 110:1, or about 105:1 to about 115:1, or about 110:1 to about 120:1, or about 115:1 to about 125:1, or about 120:1 to about 130:1, or about 125:1 to about 135:1, or about 130:1 to about 140:1, or about 135:1 to about 145:1, or about 140:1 to about 150:1, lipid:nucleic acid, weight/weight.

In some aspects, a lipid nanoparticle can comprise lipid and nucleic acid at a ratio of about 5:1, or about 10:1, or about 15:1, or about 20:1, or about 25:1, or about 30:1, or about 35:1, or about 40:1, or about 45:1, or about 50:1, or about 55:1, or about 60:1, or about 65:1, or about 70:1, or about 75:1, or about 80:1, or about 85:1, or about 90:1, or about 95:1, or about 100:1, or about 105:1, or about 110:1, or about 115:1, or about 120:1, or about 125:1, or about 130:1, or about 135:1, or about 140:1, or about 145:1, or about 150:1, or about 200:1, lipid:nucleic acid, weight/weight.

In some aspects, a lipid nanoparticle can comprise lipid and nucleic acid at a ratio of about 10:1, or about 17.5:1, or about 25:1, lipid:nucleic acid, weight/weight.

In some aspects, a nucleic acid molecule can be an RNA molecule. Thus, in some aspects, a lipid nanoparticle can comprise at least one RNA molecule. In some aspects, an RNA molecule can be an mRNA molecule. In some aspects, an mRNA molecule can comprise a 5 ‘-CAP.

In some aspects, an mRNA molecule can be capped using any method and/or capping moiety known in the art. An mRNA molecule can be capped with m7G (5’) ppp (5′) G moiety. A m7G (5′) ppp (5′) G moiety is also referred to herein as a “CapO”. An mRNA molecule can be capped with a CleanCap® moiety. A CleanCap® moiety can comprise a m7G (5′) ppp (5′) (2′OMeA) (CleanCap® AG) moiety. A CleanCap® moiety can comprise a m7G (5′) ppp (5′) (2′OMeG) (CleanCap® GG) moiety. An mRNA molecule can be capped with an anti-reverse cap analog (ARCA®) moiety. An ARCA® moiety can comprise a m7 (3′-O-methyl) G (5′) ppp (5′) G moiety. An mRNA molecule can be capped with a CleanCap® 3′OMe moiety (CleanCap®+ARCA®).

In some aspects, an mRNA molecule can comprise at least one modified nucleic acid.

Modified nucleic acids can include, but are not limited to, 5-methoxy uridine (5moU), N₁-methyl pseudouridine (me¹ψ), pseudouridine (Y), 5-methylcytidine (5-MeC).

In some aspects, a nucleic acid molecule can be a DNA molecule. Thus, in some aspects, a lipid nanoparticle can comprise at least one DNA molecule. In some aspects, a DNA molecule can be a circular DNA molecule, such as, but not limited to, a DNA plasmid. In some aspects, a lipid nanoparticle can comprise a DNA plasmid. In some aspects, a DNA molecule can be a linearized DNA molecule, such as, but not limited to, a linearized DNA plasmid. In some aspect, a DNA molecule can be a DoggyBone DNA molecule. In some aspects, a DNA molecule can be a DNA nanoplasmid.

A DNA plasmid can comprise can be at least about 0.25 kb, or at least about 0.5 kb, or at least about 0.75 kb, or at least about 1.0 kb, or at least about 1.25 kb, or at least about 1.5 kb, or at least about 1.75 kb, or at least about 2.0 kb, or at least about 2.25 kb, or at least about 2.5 kb, or at least about 2.75 kb, or at least about 3.0 kb, or at least about 3.25 kb, or at least about 3.5 kb, or at least about 3.75 kb, or at least about 4.0 kb, or at least about 4.25 kb, or at least about 4.5 kb, or at least about 4.75 kb, or at least about 5.0 kb, or at least about 5.25 kb, or at least about 5.5 kb, or at least about 5.75 kb, or at least about 6.0 kb, or at least about 6.25 kb, or at least about 6.5 kb, or at least about 6.75 kb, or at least about 7.0 kb, or at least about 7.25 kb, or at least about 7.5 kb, or at least about 7.75 kb, or at least about 8.0 kb, or at least about 8.25 kb, or at least about 8.5 kb, or at least about 8.75 kb, or at least about 9.0 kb, or at least about 9.25 kb, or at least about 9.5 kb, or at least about 9.75 kb, or at least about 10.0 kb, or at least about 10.25 kb, or at least about 10.5 kb, or at least about 10.75 kb, or at least about 11.0 kb, or at least about 11.25 kb, or at least about 11.5 kb, or at least about 11.75 kb, or at least about 12 kb, or at least about 12.25 kb, or at least about 12.5 kb, or at least about 12.75 kb, or at least about 13.0 kb, or at least about 13.25 kb, or at least about 13.5 kb, or at least about 13.75 kb, or at least about 14.0 kb, or at least about 14.25 kb, or at least about 14.5 kb, or at least about 14.75 kb or at least about 15.0 kb in length.

### LNP Compositions

In some aspects, a lipid nanoparticle can comprise at least one nucleic acid molecule, at least one bioreducible ionizable cationic lipid, and at least one structural lipid. In some aspects, a lipid nanoparticle can comprise at least one nucleic acid molecule, at least one bioreducible ionizable cationic lipid, and at least one PEGylated lipid. In some aspects, the at least one bioreducible ionizable cationic lipid can be ssPalmO-Ph-P4C2.

In some aspects, the at least one structural lipid can be a mixture of two structural lipids. In some aspects, the at least one PEGylated lipid can be a mixture of two PEGylated lipids.

In some aspects, a lipid nanoparticle can comprise at least one nucleic acid molecule, at least one bioreducible ionizable cationic lipid, at least one structural lipid, at least one PEGylated lipid or any combination thereof. In some aspects, the at least one bioreducible ionizable cationic lipid can be ssPalmO-Ph-P4C2.

In some aspects, a lipid nanoparticle can comprise at least one nucleic acid molecule, at least one bioreducible ionizable cationic lipid, at least one structural lipid, and at least one PEGylated lipid. In some aspects, the at least one bioreducible ionizable cationic lipid can be ssPalmO-Ph-P4C2.

In some aspects, a lipid nanoparticle can comprise at least one nucleic acid molecule, at least one bioreducible ionizable cationic lipid, at least one structural lipid, at least one phospholipid, at least one PEGylated lipid or any combination thereof. In some aspects, the at least one bioreducible ionizable cationic lipid can be ssPalmO-Ph-P4C2.

In some aspects, a lipid nanoparticle can comprise at least one nucleic acid molecule, at least one bioreducible ionizable cationic lipid, at least one structural lipid, at least one phospholipid and at least one PEGylated lipid. In some aspects, the at least one bioreducible ionizable cationic lipid can be ssPalmO-Ph-P4C2.

In some aspects, the nucleic acid molecule is a RNA molecule. Thus, in some aspects, a lipid nanoparticle comprising at least one nucleic acid molecule can comprise about 54% ssPalmO-Ph-P4C2 by moles, about 35% cholesterol by moles, about 5% of DOPC by moles, about 5% DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise between about 44% to 64% of ssPalmO-Ph-P4C2 by moles; between about 25% to 45% of cholesterol by moles, between about 0.1% to 20% of DOPC by moles, between about 0.1% to 20% of DSPC by moles, and between about 0.1% to 11% of DMG-PEG2000 by moles, wherein the at least one lipid nanoparticle comprises at least one nucleic acid molecule, wherein the at least one nucleic acid molecule comprises at least one RNA molecule. In some aspects, a lipid nanoparticle comprising at least one nucleic acid molecule can comprise between about 49% to 59% of ssPalmO-Ph-P4C2 by moles; between about 30% to 40% of cholesterol by moles, between about 5% to 15% of DOPC by moles, between about 5% to 15% of DSPC by moles, and between about 0.5% to 6% of DMG-PEG2000 by moles, wherein the at least one lipid nanoparticle comprises at least one nucleic acid molecule, wherein the at least one nucleic acid molecule comprises at least one RNA molecule. In some aspects, the mRNA molecule further comprises a 5′-CAP. In some aspects, the ratio of lipid to nucleic acid in the at least one nanoparticle can be about 75:1 to about 100:1 (w/w).

In some aspects, the nucleic acid molecule is a DNA molecule. Thus, the present disclosure provides a lipid nanoparticle comprising at least one nucleic acid molecule can comprise about 54% ssPalmO-Ph-P4C2 by moles, about 35% cholesterol by moles, about 5% of DOPC by moles, about 5% DSPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA (see WO/2020/154645). In some aspects, the ratio of lipid to nucleic acid in the nanoparticle can be about 100:1 (w/w).

In some aspects, a lipid nanoparticle comprising at least one nucleic acid molecule can comprise between about 44% to 64% of ssPalmO-Ph-P4C2 by moles; between about 25% to 45% of cholesterol by moles, between about 0.1% to 20% of DOPC by moles, between about 5% to 15% of DSPC by moles, and between about 0.1% to 11% of DMG-PEG2000 by moles, wherein the at least one lipid nanoparticle comprises at least one nucleic acid molecule, wherein the at least one nucleic acid molecule comprises at least one DNA molecule. In some aspects, a lipid nanoparticle comprising at least one nucleic acid molecule can comprise between about 49% to 59% of ssPalmO-Ph-P4C2 by moles; between about 30% to 40% of cholesterol by moles, between about 5% to 10% of DOPC by moles, between about 5% to 10% of DSPC by moles and between about 0.5% to 6% of DMG-PEG2000 by moles, wherein the at least one lipid nanoparticle comprises at least one nucleic acid molecule, wherein the at least one nucleic acid molecule comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 to about 100:1 (w/w).

Exemplary LNP compositions and methods of preparing such LNP compositions useful in the methods of the present disclosure include those disclosed in International Patent Application No. PCT/US2022/017570, published as WO/2022/182792, the contents of which is incorporated herein by reference in its entirety.

Accordingly, the present disclosure provides LNP compositions comprising any one of the LNP compositions set forth in WO/2022/182792, which is incorporated herein by reference in its entirety for examples for LNPs that may be used in the compositions provided herein. In some aspects, a lipid nanoparticle comprising at least one nucleic acid molecule can comprise between about 54% to 59% ssPalmO-Ph-P4C2 by moles, between about 30% to 40% cholesterol by moles, between about 5% to 10% of DOPC, DSPC or DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 to about 100:1 (w/w). In some aspects, the one mRNA encodes Cas-CLOVER. In some aspects, the lipid nanoparticle further comprises at least one gRNA. In some aspects, the lipid nanoparticle further comprises at least one gRNA pair, comprising a left gRNA and a right gRNA.

In some aspects, a lipid nanoparticle comprising at least one nucleic acid molecule can comprise about 54% ssPalmO-Ph-P4C2 by moles, about 35% cholesterol by moles, about 10% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w). In some aspects, the one mRNA encodes Cas-CLOVER. In some aspects, the lipid nanoparticle further comprises at least one gRNA. In some aspects, the lipid nanoparticle further comprises at least one gRNA pair, comprising a left gRNA and a right gRNA.

In some aspects, a lipid nanoparticle comprising at least one nucleic acid molecule can comprise about 54% ssPalmO-Ph-P4C2 by moles, about 35% cholesterol by moles, about 10% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w). In some aspects, the one mRNA encodes Cas-CLOVER. In some aspects, the lipid nanoparticle further comprises at least one gRNA. In some aspects, the lipid nanoparticle further comprises at least one gRNA pair, comprising a left gRNA and a right gRNA.

In some aspects, a lipid nanoparticle comprising at least one nucleic acid molecule can comprise about 56.5% ssPalmO-Ph-P4C2 by moles, about 32.5% cholesterol by moles, about 10% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w). In some aspects, the one mRNA encodes Cas-CLOVER. In some aspects, the lipid nanoparticle further comprises at least one gRNA. In some aspects, the lipid nanoparticle further comprises at least one gRNA pair, comprising a left gRNA and a right gRNA.

In some aspects, a lipid nanoparticle comprising at least one nucleic acid molecule can comprise about 56.5% ssPalmO-Ph-P4C2 by moles, about 32.5% cholesterol by moles, about 10% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w). In some aspects, the one mRNA encodes Cas-CLOVER. In some aspects, the lipid nanoparticle further comprises at least one gRNA. In some aspects, the lipid nanoparticle further comprises at least one gRNA pair, comprising a left gRNA and a right gRNA.

In some aspects, a lipid nanoparticle comprising at least one nucleic acid molecule can comprise about 59% ssPalmO-Ph-P4C2 by moles, about 30% cholesterol by moles, about 10% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w). In some aspects, the one mRNA encodes Cas-CLOVER. In some aspects, the lipid nanoparticle further comprises at least one gRNA. In some aspects, the lipid nanoparticle further comprises at least one gRNA pair, comprising a left gRNA and a right gRNA.

In some aspects, a lipid nanoparticle comprising at least one nucleic acid molecule can comprise about 59% ssPalmO-Ph-P4C2 by moles, about 30% cholesterol by moles, about 10% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w). In some aspects, the one mRNA encodes Cas-CLOVER. In some aspects, the lipid nanoparticle further comprises at least one gRNA. In some aspects, the lipid nanoparticle further comprises at least one gRNA pair, comprising a left gRNA and a right gRNA.

In some aspects, a lipid nanoparticle comprising at least one nucleic acid molecule can comprise about 54% ssPalmO-Ph-P4C2 by moles, about 40% cholesterol by moles, about 5% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w). In some aspects, the one mRNA encodes Cas-CLOVER. In some aspects, the lipid nanoparticle further comprises at least one gRNA. In some aspects, the lipid nanoparticle further comprises at least one gRNA pair, comprising a left gRNA and a right gRNA.

In some aspects, a lipid nanoparticle comprising at least one nucleic acid molecule can comprise about 54% ssPalmO-Ph-P4C2 by moles, about 40% cholesterol by moles, about 5% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w). In some aspects, the one mRNA encodes Cas-CLOVER. In some aspects, the lipid nanoparticle further comprises at least one gRNA. In some aspects, the lipid nanoparticle further comprises at least one gRNA pair, comprising a left gRNA and a right gRNA.

In some aspects, a lipid nanoparticle comprising at least one nucleic acid molecule can comprise about 56.5% ssPalmO-Ph-P4C2 by moles, about 37.5% cholesterol by moles, about 5% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w). In some aspects, the one mRNA encodes Cas-CLOVER. In some aspects, the lipid nanoparticle further comprises at least one gRNA. In some aspects, the lipid nanoparticle further comprises at least one gRNA pair, comprising a left gRNA and a right gRNA.

In some aspects, a lipid nanoparticle comprising at least one nucleic acid molecule can comprise about 56.5% ssPalmO-Ph-P4C2 by moles, about 37.5% cholesterol by moles, about 5% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w). In some aspects, the one mRNA encodes Cas-CLOVER. In some aspects, the lipid nanoparticle further comprises at least one gRNA. In some aspects, the lipid nanoparticle further comprises at least one gRNA pair, comprising a left gRNA and a right gRNA.

In some aspects, the lipid nanoparticles of the present disclosure can be produced using a microfluidic-mixing platform. In some aspects, the microfluidic-mixing platform can be a non-turbulent microfluidic mixing platform.

In some aspects, a microfluidic-mixing platform can produce the lipid nanoparticles of the present disclosure by combining a miscible solvent phase comprising the lipid components of the nanoparticle and an aqueous phase comprising the lipid nanoparticle cargo (e.g., nucleic acid, DNA, mRNA, etc.) using a microfluidic device. In some aspects, the miscible solvent phase and the aqueous phase are mixed in the microfluidic device under laminar flow conditions that do not allow for immediate mixing of the two phases. As the two phases move under laminar flow in a microfluidic channel, microscopic features in the channel can allow for controlled, homogenous mixing to produce the lipid nanoparticles of the present disclosure.

In some aspects, the microfluidic-mixing platform can include, but are not limited to the NanoAssemblr® Spark (Precision NanoSystems), the NanoAssemblr® Ignite™ (Precision NanoSystems), the NanoAssemblr® Benchtop (Precision NanoSystems), the NanoAssemblr® Blaze (Precision NanoSystems) or the NanoAssemblr® GMP System (Precision NanoSystems).

In some aspects, the lipid nanoparticles of the present disclosure can be produced using a microfluidic-mixing platform, wherein the microfluidic mixing platform mixes at a rate of at least about 2.5 ml/min, or at least about 5 ml/min, or at least about 7.5 ml/min, or at least about 10 ml/min, or at least about 12.5 ml/min, or at least about 15 ml/min, or at least about 17.5 ml/min, or at least about 20 ml/min, or at least about 22.5 ml/min, or at least about 25 ml/min, or at least about 27.5 ml/min, or at least about 30 ml/min.

In some aspects, the lipid nanoparticles of the present disclosure can be produced using a T-mixer, wherein the T-mixer mixes at a rate of at least about 2.5 ml/min, or at least about 5 ml/min, or at least about 7.5 ml/min, or at least about 10 ml/min, or at least about 12.5 ml/min, or at least about 15 ml/min, or at least about 17.5 ml/min, or at least about 20 ml/min, or at least about 22.5 ml/min, or at least about 25 ml/min, or at least about 27.5 ml/min, or at least about 30 ml/min.

In some aspects, the lipid nanoparticles of the present disclosure can be produced using a microfluidic-mixing platform, wherein the microfluidic mixing platform mixes a miscible solvent phase and an aqueous phase at a ratio of about 10:1, or about 9:1, or about 8:1, or about 7:1, or about 6:1, or about 5:1, or about 4:1, or about 3:1, or about 2:1, or about 1:1, or about 1:2, or about 1:3, or about 1:4, or about 1:5, or about 1:6, or about 1:7, or about 1:8, or about 1:9, or about 1:10, solvent:aqueous, v:v.

In some aspects, the lipid nanoparticles of the present disclosure can be produced using a T-mixer, wherein the T-mixer mixes a miscible solvent phase and an aqueous phase at a ratio of about 10:1, or about 9:1, or about 8:1, or about 7:1, or about 6:1, or about 5:1, or about 4:1, or about 3:1, or about 2:1, or about 1:1, or about 1:2, or about 1:3, or about 1:4, or about 1:5, or about 1:6, or about 1:7, or about 1:8, or about 1:9, or about 1:10, solvent:aqueous, v:v.

### Adeno-Associated Virus (AAV) Donor Polynucleotides

The present disclosure provides compositions comprising adeno-associated virus (AAV) donor polynucleotides.

In some aspects, an AAV donor polynucleotide comprising a promoterless, bidirectional luciferase expression cassette can comprise at least one AAV inverted terminal repeat (ITR) sequence. In some aspects, an AAV donor polynucleotide can comprise at least a first and a second targeting sequences comprising a nucleic acid sequence homologous to genomic sequences in the genome of a cell. In some aspects, an AAV donor polynucleotide can comprise at least one splice sequence. In some aspects an AAV donor polynucleotide can comprise at least one P2A sequence. In some aspects an AAV donor polynucleotide can comprise at least one luciferase transgene sequence. In some aspects, an AAV donor polynucleotide can comprise at least one poly(A) sequence. In some aspects, an AAV donor polynucleotide can comprise at least one DNA spacer sequence.

An AAV donor polynucleotide can comprise a first AAV ITR sequence, a first target sequence comprising a nucleic acid sequence homologous to a first genomic sequence in the genome of a cell, a first splice acceptor sequence, a luciferase coding sequence, a poly(A) sequence, a second target sequence comprising a nucleic acid sequence homologous to a second genomic sequence in the genome of a cell; and a second AAV ITR sequence.

An AAV donor polynucleotide can comprise a first AAV ITR sequence, a first target sequence comprising a nucleic acid sequence homologous to a first genomic sequence in the genome of a cell, a first splice acceptor sequence, a P2A sequence, a luciferase coding sequence, a poly(A) sequence, a second target sequence comprising a nucleic acid sequence homologous to a second genomic sequence in the genome of a cell; and a second AAV ITR sequence.

An AAV donor polynucleotide can comprise a first AAV ITR sequence, a first target sequence comprising a nucleic acid sequence homologous to a first genomic sequence in the genome of a cell, a first splice acceptor sequence, a P2A sequence, a luciferase coding sequence, a poly(A) sequence, a DNA spacer sequence; an inverted poly(A) sequence, and inverted luciferase coding sequence, an inverted P2A sequence, an inverted splice acceptor sequence, a second target sequence comprising a nucleic acid sequence homologous to a second genomic sequence in the genome of a cell; and a second AAV ITR sequence.

An AAV donor polynucleotide can comprise a first AAV ITR sequence, a first target sequence comprising a nucleic acid sequence homologous to a first sequence of intron 3 of the albumin gene, a first splice acceptor sequence, a P2A sequence, a luciferase coding sequence, a poly(A) sequence, a DNA spacer sequence; an inverted poly(A) sequence, and inverted luciferase coding sequence, an inverted P2A sequence, an inverted splice acceptor sequence, a second target sequence comprising a nucleic acid sequence homologous to a second sequence of intron 3 of the albumin gene; and a second AAV ITR sequence.

In some aspects, an AAV donor polynucleotide comprising a promoterless, bidirectional PAH expression cassette can comprise at least one AAV inverted terminal repeat (ITR) sequence. In some aspects, an AAV donor polynucleotide can comprise at least a first target sequence and a second target sequences comprising a nucleic acid sequence homologous to a first and a second genomic sequence in the genome of a cell. In some aspects, an AAV donor polynucleotide can comprise at least one splice sequence. In some aspects an AAV donor polynucleotide can comprise at least one P2A sequence. In some aspects an AAV donor polynucleotide can comprise at least one PAH transgene sequence. In some aspects, an AAV donor polynucleotide can comprise at least one poly(A) sequence. In some aspects, an AAV donor polynucleotide can comprise at least one DNA spacer sequence.

An AAV donor polynucleotide can comprise a first AAV ITR sequence, a first target sequence comprising a nucleic acid sequence homologous to a first genomic sequence in the genome of a cell, a first splice acceptor sequence, a PAH coding sequence, a poly(A) sequence, a second targeting sequence comprising a nucleic acid sequence homologous to a second genomic sequence in the genome of a cell; and a second AAV ITR sequence.

An AAV donor polynucleotide can comprise a first AAV ITR sequence, a first target sequence comprising a nucleic acid sequence homologous to a first genomic sequence in the genome of a cell, a first splice acceptor sequence, a P2A sequence, a PAH coding sequence, a poly(A) sequence, a second target sequence comprising a nucleic acid sequence homologous to a second genomic sequence in the genome of a cell; and a second AAV ITR sequence.

An AAV donor polynucleotide can comprise a first AAV ITR sequence, a first target sequence comprising a nucleic acid sequence homologous to a first genomic sequence in the genome of a cell, a first splice acceptor sequence, a P2A sequence, a PAH coding sequence, a poly(A) sequence, a DNA spacer sequence; an inverted poly(A) sequence, and inverted PAH coding sequence, an inverted P2A sequence, an inverted splice acceptor sequence, a second target sequence comprising a nucleic acid sequence homologous to a second genomic sequence in the genome of a cell; and a second AAV ITR sequence.

An AAV donor polynucleotide can comprise a first AAV ITR sequence, a first target sequence comprising a nucleic acid sequence homologous to a first sequence of intron 3 of the albumin gene, a first splice acceptor sequence, a P2A sequence, a PAH coding sequence, a poly(A) sequence, a DNA spacer sequence; an inverted poly(A) sequence, and inverted PAH coding sequence, an inverted P2A sequence, an inverted splice acceptor sequence, a second target sequence comprising a nucleic acid sequence homologous to a second sequence of intron 3 of the albumin gene; and a second AAV ITR sequence.

In some aspects, an AAV donor polynucleotide comprising a PAH expression cassette can comprise An AAV donor polynucleotide can comprise a first AAV ITR sequence, a first target sequence comprising a nucleic acid sequence homologous to a first genomic sequence in the genome of a cell, at least one promoter sequence, a PAH coding sequence, a poly(A) sequence, a second target sequence comprising a nucleic acid sequence complementary to a second genomic sequence in the genome of a cell; and a second AAV ITR sequence.

In some aspects, an AAV donor polynucleotide comprising a PAH expression cassette can comprise an AAV donor polynucleotide can comprise a first AAV ITR sequence, a first target sequence comprising a nucleic acid sequence homologous to a first genomic sequence in the genome of a cell, a TTRe promoter sequence, a PAH coding sequence, a poly(A) sequence, a second target sequence comprising a nucleic acid sequence homologous to a second genomic sequence in the genome of a cell; and a second AAV ITR sequence.

In some aspects, an AAV donor polynucleotide comprising a PAH expression cassette can comprise An AAV donor polynucleotide can comprise a first AAV ITR sequence, a first target sequence comprising a nucleic acid sequence homologous to a first sequence of intron 3 of the albumin gene, a TTRe promoter sequence, a PAH coding sequence, a poly(A) sequence, a second target sequence comprising a nucleic acid sequence homologous to a second sequence of intron 3 of the albumin gene; and a second AAV ITR sequence

In a non-limiting example of the preceding AAV donor polynucleotides, the at least one transgene sequence can comprise a nucleic acid sequence that encodes a human phenylalanine hydroxylase (hPAH) polypeptide. In certain aspects, the nucleotide sequence encoding hPAH is codon optimized. In certain aspects, the codon optimized PAH gene is further modified to i) any undesired restriction enzyme recognition sites; and ii) putative cryptic splice sites for cloning the modified sequences into AAV donor vectors. These non-limiting examples of AAV donor polynucleotides are shown in FIGS. 1B and 1C.

In some aspects, an AAV donor polynucleotide comprising a promoterless, bidirectional luciferase expression cassette can comprise, consist essentially of or consist of a nucleic acid sequence at least 65%, 70%, 75%, 80%, 85%, 90%, 95%, 96%, 97%, 98%, or 99% (or any percentage in between) identical to the sequence set forth in SEQ ID NO: 6. In some aspects, an AAV donor polynucleotide comprising a promoterless, bidirectional luciferase expression cassette can comprise, consist essentially of or consist of the sequence set forth in SEQ ID NO: 6.

In some aspects, an AAV donor polynucleotide comprising a promoterless, bidirectional PAH expression cassette can comprise, consist essentially of or consist of a nucleic acid sequence at least 65%, 70%, 75%, 80%, 85%, 90%, 95%, 96%, 97%, 98%, or 99% (or any percentage in between) identical to the sequence set forth in SEQ ID NO: 8. In some aspects, an AAV donor polynucleotide comprising a promoterless, bidirectional PAH expression cassette can comprise, consist essentially of or consist of the sequence set forth in SEQ ID NO: 8.

In some aspects, an AAV donor polynucleotide comprising a PAH TTRe expression cassette can comprise, consist essentially of or consist of a nucleic acid sequence at least 65%, 70%, 75%, 80%, 85%, 90%, 95%, 96%, 97%, 98%, or 99% (or any percentage in between) identical to the sequence set forth in SEQ ID NO: 10. In some aspects, an AAV donor polynucleotide comprising a PAH TTRe expression cassette can comprise, consist essentially of or consist of the sequence set forth in SEQ ID NO: 10.

AAV ITR Sequences

The vectors described herein may comprise one or more AAV ITR sequences. An AAV ITR sequence can comprise any suitable AAV ITR sequence known in the art or described herein. In some embodiments, the AAV ITR sequence is an AAV1, AAV2, AAV3, AAV4, AAV5, AAV6, AAV7, AAV8, AAV9, AAV10 or AAV11 ITR sequence. In some aspects, an AAV ITR sequence can comprise, consist essentially of, or consist of a nucleic acid sequence at least 65%, 70%, 75%, 80%, 85%, 90%, 95%, 96%, 97%, 98%, or 99% (or any percentage in between) identical to any one of the sequences set forth in SEQ ID NOs: 12 and 24. In some aspects, an AAV ITR sequence can comprise, consist essentially of, or consist any one of the sequences set forth in SEQ ID NOs: 12 and 24.

In some aspects, a first AAV ITR sequence can comprise consist essentially of, or consist of a nucleic acid sequence at least 65%, 70%, 75%, 80%, 85%, 90%, 95%, 96%, 97%, 98%, or 99% (or any percentage in between) identical to SEQ ID NO: 12 and a second AAV ITR sequence can comprise consist essentially of, or consist of a nucleic acid sequence at least 65%, 70%, 75%, 80%, 85%, 90%, 95%, 96%, 97%, 98%, or 99% (or any percentage in between) identical to SEQ ID NO: 24. In some aspects, a first AAV ITR sequence can comprise consist essentially of, or consist of the nucleic acid sequence of SEQ ID NO: 12 and a second AAV ITR sequence can comprise consist essentially of, or consist of the nucleic acid sequence of SEQ ID NO: 24.

### Splice Acceptor Sequences

In some aspects, the AAV donor polynucleotides comprise one or more splice acceptor sequence to facilitate expression of transgenes integrated into the intron of a gene. In some aspects, a splice acceptor sequence can comprise any suitable splice acceptor sequence known in the art, or derived from mammalian or viral splice acceptor sequences.

In some aspects, a splice acceptor sequence can comprise, consist essentially of, or 98%, or 99% (or any percentage in between) identical to the sequence set forth in SEQ ID NOs: 14 or 22. In some aspects, a splice acceptor sequence can comprise, consist essentially of, or consist of the nucleic acid sequence set forth in SEQ ID NOs: 14 or 22.

### Promoter Sequences

In some aspects, a promoter sequence can comprise any suitable promoter sequence known in the art. In some aspects, a promoter sequence can comprise any liver-specific promoter sequence known in the art.

In some aspects, a promoter sequence can comprise, consist essentially of, or consist of a nucleic acid sequence at least 65%, 70%, 75%, 80%, 85%, 90%, 95%, 96%, 97%, 98%, or 99% (or any percentage in between) identical to the sequence set forth in SEQ ID NO: 27. In some aspects, a promoter sequence can comprise, consist essentially of, or consist of the sequence set forth in SEQ ID NO: 27.

In some aspects, a promoter sequence can comprise a TTRe promoter sequence. A TTRe promoter sequence can comprise, consist essentially of, or consist of a nucleic acid sequence at least 65%, 70%, 75%, 80%, 85%, 90%, 95%, 96%, 97%, 98%, or 99% (or any percentage in between) identical to SEQ ID NO: 27. A TTRe promoter sequence can comprise, consist essentially of, or consist of the sequence set forth in SEQ ID NO: 27.

### Transgene Sequences

In some aspects, a transgene sequence can comprise a nucleic acid sequence that encodes a human phenylalanine hydroxylase (hPAH) polypeptide. In some aspects, a nucleic acid sequence that encodes a hPAH polypeptide can comprise, consist essentially of, or consist of a nucleic acid sequence at least 65%, 70%, 75%, 80%, 85%, 90%, 95%, 96%, 97%, 98%, or 99% (or any percentage in between) identical to SEQ ID NO: 25. In some aspects, a nucleic acid sequence that encodes a hPAH polypeptide can comprise, consist essentially of, or consist of the nucleic acid sequence set forth in SEQ ID NO: 25.

In some aspects, a transgene sequence can comprise a tag, for example a hemagglutinin (HA) tag. Such tags are well known in the art and useful for protein purification.

In some aspects, a transgene sequence can be codon optimized according to methods known in the art.

In some aspects, the nucleic acid sequence encoding a polypeptide (e.g. hPAH) can be a codon optimized nucleic acid sequence that encodes the polypeptide. A codon optimized nucleic acid sequence encoding a polypeptide can comprise, consist essentially of, or consist of a nucleic acid sequence that is no more than 65%, 70%, 75%, 80%, 85%, 90%, 95%, 96%, 97%, 98%, or 99% (or any percentage in between) identical to the wildtype human nucleic acid sequence encoding the polypeptide.

SEQ ID NO: 25 is a unique codon optimized PAH nucleic acid sequence that can be included in the polynucleotides, vectors and compositions of the present disclosure.

In some aspects, a codon optimized nucleic acid sequence encoding a polypeptide, such as that set forth in SEQ ID NO: 25, can comprise no splice sites in the donor DNA. In some aspects, a codon optimized nucleic acid sequence encoding a polypeptide can comprise no more than about one, or about two, or about three, or about four, or about five, or about six, or about seven, or about eight, or about nine, or about ten splice sites. In some aspects, a codon optimized nucleic acid sequence encoding a polypeptide comprises at least one, or at least two, or at least three, or at least four, or at least five, or at least six, or at least seven, or at least eight, or at least nine, or at least ten fewer splice sites as compared to the wildtype human nucleic acid sequence encoding the polypeptide. The removal of splice sites in the codon optimized nucleic acid sequence donor DNA was found to unexpectedly and unpredictably increase expression of the polypeptide in vivo, as cryptic splicing is prevented. Moreover, cryptic splicing may vary between different subjects, meaning that the expression level of the polypeptide comprising donor splice sites may unpredictably vary between different subjects.

In some aspects, a codon optimized nucleic acid sequence encoding a polypeptide, such as that set forth in SEQ ID NO: 25, can have a GC content that differs from the GC content of the wildtype human nucleic acid sequence encoding the polypeptide. In some aspects, the GC content of a codon optimized nucleic acid sequence encoding a polypeptide is more evenly distributed across the entire nucleic acid sequence, as compared to the wildtype human nucleic acid sequence encoding the polypeptide. Without wishing to be bound by theory, by more evenly distributing the GC content across the entire nucleic acid sequence, the codon optimized nucleic acid sequence is expected to exhibit a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects, the codon optimized nucleic acid sequence encoding a polypeptide, such as that set forth in SEQ ID NO: 25, exhibits at least 5%, at least 10%, at least 20%, at least 30%, at least 50%, at least 75%, at least 100%, at least 200%, at least 300%, at least 500%, or at least 1000% increased expression in a human subject relative to a wild-type or non-codon optimized nucleic acid sequence encoding the polypeptide.

In some aspects, at least one transgene sequence can be operatively linked to at least one promoter sequence present in the same polynucleotide.

### Poly(A) Sequences

In some aspects, the AAV donor polynucleotides comprise a poly(A) sequence. In some aspects, the AAV donor polynucleotides comprise an inverted poly(A) sequence. In some aspects, the AAV donor polynucleotides comprise a poly(A) sequence and an inverted polyA sequence.

In some aspects, a poly(A) sequence can comprise any poly(A) sequence known in the art or described herein. Non-limiting examples of poly(A) sequences include, but are not limited to, SV40 poly(A) sequences and bGH poly(A) sequences. In some aspects, the poly(A) sequence can comprise, consist essentially of, or consist of a nucleic acid sequence at least 65%, 70%, 75%, 80%, 85%, 90%, 95%, 96%, 97%, 98%, or 99% (or any percentage in between) identical to the sequence set forth in SEQ ID NO: 17. In some aspects, the poly(A) sequence can comprise, consist essentially of, or consist of the nucleic acid sequence set forth in SEQ ID NO: 17. In some aspects, the inverted poly(A) sequence can comprise, consist essentially of, or consist of a nucleic acid sequence at least 65%, 70%, 75%, 80%, 85%, 90%, 95%, 96%, 97%, 98%, or 99% (or any percentage in between) identical to the sequence set forth in SEQ ID NO: 19. In some aspects, the inverted poly(A) sequence can comprise, consist essentially of, or consist of the nucleic acid sequence set forth in SEQ ID NO: 19.

### DNA Spacer Sequences

In some aspects, the AAV donor polynucleotides comprises one or more spacer sequences. In some aspects, a DNA spacer sequence can comprise, consist essentially of or 98%, or 99% (or any percentage in between) identical to the sequences set forth in SEQ ID NO: 18.

DNA spacer sequences can be located at any position within an AAV donor polynucleotide. In some aspects, the DNA spacer sequence is positioned between the poly(A) sequence and the inverted poly(A) sequence.

### Enhancer Sequences

In some aspects, the AAV donor polynucleotides comprises one or more enhancer sequences. In some aspects, an enhancer sequence can comprise any suitable enhancer sequence known in the art. In some aspects, an enhancer sequence can comprise any suitable liver-specific enhancer sequence known in the art.

In some aspects, an enhancer sequence can comprise, consist essentially of, or consist of a nucleic acid sequence at least 65%, 70%, 75%, 80%, 85%, 90%, 95%, 96%, 97%, 98%, or 99% (or any percentage in between) identical to any one of the sequences set forth in SEQ ID NOs: 5 and 6. In some aspects, an enhancer sequence can comprise, consist essentially of, or consist of the nucleic acid sequence set forth in SEQ ID NOs: 5 or 6.

In some aspects, an enhancer sequence can comprise a TTR enhancer sequence. A TTR enhancer sequence can comprise, consist essentially of, or consist of a nucleic acid sequence at least 65%, 70%, 75%, 80%, 85%, 90%, 95%, 96%, 97%, 98%, or 99% (or any percentage in between) identical to SEQ ID NO: 6. A TTR enhancer sequence can comprise, consist essentially of, or consist of the nucleic acid sequence set forth in SEQ ID NO: 6.

### 3′ UTR Sequences

In some aspects, the AAV donor polynucleotides comprises a 3′ untranslated region (UTR) sequence. In some aspects, a 3′ UTR sequence can comprise, consist essentially of or consist of a nucleic acid sequence at least 65%, 70%, 75%, 80%, 85%, 90%, 95%, 96%, 97%, 98%, or 99% (or any percentage in between) identical to the sequence set forth in SEQ ID NO: 10. In some aspects, a 3′ UTR sequence can comprise, consist essentially of or consist of the nucleic acid sequence set forth in SEQ ID NO: 10.

In some aspects of the preceding transposons, a first 3′ UTR sequence can be an AES-mtRNR 3′ UTR sequence. An AES-mtRNR 3′ UTR sequence can comprise, consist essentially of, or consist of a nucleic acid sequence at least 65%, 70%, 75%, 80%, 85%, 90%, 95%, 96%, 97%, 98%, or 99% (or any percentage in between) identical to SEQ ID NO: 10, or can comprise any suitable UTR sequences known in the art, particularly those known to enhance translation and/or stabilize an RNA by slowing its degradation. In some embodiments, the first 3′UTR sequence comprises the nucleic acid sequence set forth in SEQ ID NO: 10.

Illustrative dCas9-Clo051 (Cas-CLOVER) Fusion Proteins

In another aspect, provided herein are fusion proteins comprising a nuclease or a nuclease domain and an endonuclease. The nuclease or the nuclease domain thereof can comprise a nuclease-inactivated Cas (dCas) protein and an endonuclease. The endonuclease can comprise a Clo051 nuclease or a nuclease domain thereof. The gene editing composition can comprise a fusion protein. The fusion protein can comprise a nuclease-inactivated Cas9 (dCas9) protein and a Clo051 nuclease or a Clo051 nuclease domain. The gene editing composition can further comprise a guide sequence. The guide sequence comprises an RNA sequence.

The disclosure provides compositions comprising a small, Cas9 (Cas9) operatively linked to an effector. The disclosure provides a fusion protein comprising, consisting essentially of or consisting of a DNA localization component and an effector molecule, wherein the effector comprises a small, Cas9 (Cas9). A small Cas9 construct of the disclosure can comprise an effector comprising a type IIS endonuclease. A Staphylococcus aureus Cas9 with an active catalytic site may comprise the amino acid sequence of SEQ ID NO: 30.

The disclosure provides compositions comprising an inactivated, small, Cas9 (dSaCas9) operatively linked to an effector. The disclosure provides a fusion protein comprising a DNA localization component and an effector molecule, wherein the effector comprises a small, inactivated Cas9 (dSaCas9). A small, inactivated Cas9 (dSaCas9) construct of the disclosure can comprise an effector comprising a type IIS endonuclease. In some embodiments, the dSaCas9 comprises the amino acid sequence of SEQ ID NO: 31, which includes a D10A and a N580A mutation to inactivate the catalytic site. In some embodiments, the dSaCas9 comprises a sequence that is at least at least 65%, 70%, 75%, 80%, 85%, 90%, 95%, 96%, 97%, 98%, or 99% identical to the sequence set forth in SEQ ID NO: 31.

The disclosure provides compositions comprising an inactivated Cas9 (dCas9) operatively linked to an effector. The disclosure provides a fusion protein comprising a DNA localization component and an effector molecule, wherein the effector comprises an inactivated Cas9 (dCas9). An inactivated Cas9 (dCas9) construct of the disclosure can comprise an effector comprising a type IIS endonuclease.

The dCas9 can be isolated or derived from Streptococcus pyogenes. The dCas9 can comprise a dCas9 with substitutions at amino acid positions 10 and 840, which inactivate the catalytic site. In some aspects, these substitutions are D10A and H840A. The dCas9 can comprise the amino acid sequence of SEQ ID NO: 32 or SEQ ID NO: 33. In some embodiments, the dCas9 comprises a sequence that is at least at least 65%, 70%, 75%, 80%, 85%, 90%, 95%, 96%, 97%, 98%, or 99% identical to the sequence set forth in SEQ ID NOs: 32 or 33.

An exemplary Clo051 nuclease domain comprises, consists essentially of or consists of, the amino acid sequence of SEQ ID NO: 34. In some embodiments, the Clo051 nuclease domain comprises a sequence that is at least at least 65%, 70%, 75%, 80%, 85%, 90%, 95%, 96%, 97%, 98%, or 99% identical to the sequence set forth in SEQ ID NO: 34. In some aspects, the Clo051 nuclease domain comprises at least one amino acid substitution. In some aspects, the amino acid substitution is in the alpha-helix-loop domain of the Clo051 nuclease. In some aspects, the amino acid substitution is at position 35, 37, 60, 92, 98, 100 or 146 of SEQ ID NO: 34. In some aspects, the amino acid substitution is at position 37 of SEQ ID NO: 34.

An exemplary dCas9-Clo051 (Cas-CLOVER) fusion protein can comprise, consist essentially of, or consist of, the amino acid sequence of SEQ ID NO: 37. In some embodiments, the Cas-CLOVER fusion protein comprises a sequence that is at least 65%, 70%, 75%, 80%, 85%, 90%, 95%, 96%, 97%, 98%, or 99% identical to the sequence set forth in SEQ ID NO: 37. The exemplary dCas9-Clo051 fusion protein can be encoded by a polynucleotide which comprises, consists essentially of, or consists of, the nucleic acid sequence of SEQ ID NO: 38. In some embodiments, the Cas-CLOVER fusion protein is encoded by a polynucleotide comprising a sequence that is at least at least 65%, 70%, 75%, 80%, 85%, 90%, 95%, 96%, 97%, 98%, or 99% identical to the sequence set forth in SEQ ID NO: 38. The nucleic acid encoding the dCas9-Clo051 fusion protein can be DNA or RNA.

An exemplary dCas9-Clo051 (Cas-CLOVER) fusion protein can comprise, consist essentially of, or consist of, the amino acid sequence of SEQ ID NO: 39. In some embodiments, the Cas-CLOVER fusion protein comprises a sequence that is at least at least 65%, 70%, 75%, 80%, 85%, 90%, 95%, 96%, 97%, 98%, or 99% identical to the sequence set forth in SEQ ID NO: 39. The exemplary dCas9-Clo051 fusion protein can be encoded by a polynucleotide which comprises, consists essentially of, or consists of, the nucleic acid sequence of SEQ ID NO: 40. In some embodiments, the Cas-CLOVER fusion protein is encoded by a polynucleotide comprising a sequence that is at least at least 65%, 70%, 75%, 80%, 85%, 90%, 95%, 96%, 97%, 98%, or 99% identical to the sequence set forth in SEQ ID NO: 40. The nucleic acid encoding the dCas9-Clo051 fusion protein can be DNA or RNA.

An exemplary dCas9-Clo051 fusion (Cas-CLOVER) fusion protein of the disclosure may further comprise at least one nuclear localization sequence (NLS). In some embodiments, the dCas9-Clo051 fusion protein of the disclosure comprises at least two nuclear localization sequences. In some embodiments, the NLS is on the N′ terminal end of the dCas9-Clo051 fusion protein (NLS-dCas9-Clo051). In some embodiments, the NLS is on the C-terminal end of the dCas9-Clo051 fusion protein (dCas9-Clo051-NLS). In some embodiments, the NLS is on the N′ terminal end and at the C′ terminal end of the dCas9-Clo051 fusion protein (“NLS-dCas9-Clo051-NLS” or “wildtype Cas-CLOVER”).

The NLS-dCas9-Clo051-NLS (“wildtype Cas-CLOVER”) fusion protein can comprise, consist essentially of, or consist of, the amino acid sequence of SEQ ID NO: 28. In some embodiments, the NLS-dCas9-Clo051-NLS fusion protein comprises a sequence that is at least at least 65%, 70%, 75%, 80%, 85%, 90%, 95%, 96%, 97%, 98%, or 99% identical to the sequence set forth in SEQ ID NO: 28.

NLS-dCas9-Clo051-NLS amino acid sequence (NLS amino acid sequence is bolded and underlined):

(SEQ ID NO: 28)
MAPKKKRKVEGIKSNISLLKDELRGQISHISHEYLSLIDLAFDSKQNRLFE
MKVLELLVNEYGFKGRHLGGSRKPDGIVYSTTLEDNFGIIVDTKAYSEGYS
LPISQADEMERYVRENSNRDEEVNPNKWWENFSEEVKKYYFVFISGSFKGK
FEEQLRRLSMTTGVNGSAVNVVNLLLGAEKIRSGEMTIEELERAMENNSEF
ILKYGGGGSDKKYSIGLAIGTNSVGWAVITDEYKVPSKKFKVLGNTDRHSI
KKNLIGALLFDSGETAEATRLKRTARRRYTRRKNRICYLQEIFSNEMAKVD
DSFFHRLEESELVEEDKKHERHPIFGNIVDEVAYHEKYPTIYHLRKKLVDS
TDKADLRLIYLALAHMIKERGHFLIEGDLNPDNSDVDKLFIQLVQTYNQLF
EENPINASGVDAKAILSARLSKSRRLENLIAQLPGEKKNGLFGNLIALSLG
LTPNFKSNEDLAEDAKLQLSKDTYDDDLDNLLAQIGDQYADLFLAAKNLSD
AILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLLR
KQRTEDNGSIPHQIHLGELHAILRRQEDFYPFLKDNREKIEKILTFRIPYY
VGPLARGNSRFAWMTRKSEETITPWNFEEVVDKGASAQSFIERMTNFDKNL
PNEKVLPKHSLLYEYFTVYNELTKVKYVTEGMRKPAFLSGEQKKAIVDLLF
KTNRKVTVKQLKEDYFKKIECFDSVEISGVEDRENASLGTYHDLLKIIKDK
DELDNEENEDILEDIVLTLTLFEDREMIEERLKTYAHLEDDKVMKQLKRRR
YTGWGRLSRKLINGIRDKQSGKTILDFLKSDGFANRNEMQLIHDDSLTFKE
DIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQN
EKLYLYYLQNGRDMYVDQELDINRLSDYDVDAIVPQSFLKDDSIDNKVLTR
SDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSK
LVSDERKDFQFYKVREINNYHHAHDAYLNAVVGTALIKKYPKLESEFVYGD
YKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELLG
ITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLD
EIIEQISEFSKRVILADANLDKVLSAYNKHRDKPIREQAENIIHLFTLINL
GAPAAFKYFDTTIDRKRYTSTKEVLDATLIHQSITGLYETRIDLSQLGGDG
SPKKKRKVSS.

The NLS-dCas9-Clo051-NLS fusion protein can comprise, consist essentially of, or consist of, the amino acid sequence of SEQ ID NO: 3. In some embodiments, the NLS-dCas9-Clo051-NLS fusion protein comprises a sequence that is at least at least 65%, 70%, 75%, 80%, 85%, 90%, 95%, 96%, 97%, 98%, or 99% identical to the sequence set forth in SEQ ID NO: 3.

(SEQ ID NO: 3)
MAPKKKRKVEGIKSNISLLKDELRGQISHISHEYLSLIDLAFDPKQNRLFE
MKVLELLVNEYGFKGRHLGGSRKPDGIVYSTTLEDNFGIIVDTKAYSEGYS
LPISQADEMERYVRENSNRDEEVNPNKWWENFSEEVKKYYFVFISGSFKGK
FEEQLRRLSMTTGVNGSAVNVVNLLLGAEKIRSGEMTIEELERAMENNSEF
ILKYGGGGSDKKYSIGLAIGTNSVGWAVITDEYKVPSKKFKVLGNTDRHSI
KKNLIGALLFDSGETAEATRLKRTARRRYTRRKNRICYLQEIFSNEMAKVD
DSFFHRLEESELVEEDKKHERHPIFGNIVDEVAYHEKYPTIYHLRKKLVDS
TDKADLRLIYLALAHMIKERGHFLIEGDLNPDNSDVDKLFIQLVQTYNQLF
EENPINASGVDAKAILSARLSKSRRLENLIAQLPGEKKNGLFGNLIALSLG
LTPNEKSNEDLAEDAKLQLSKDTYDDDLDNLLAQIGDQYADLFLAAKNLSD
AILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKNL
PNEKVLPKHSLLYEYFTVYNELTKVKYVTEGMRKPAFLSGEQKKAIVDLLF
KTNRKVTVKQLKEDYFKKIECFDSVEISGVEDRFNASLGTYHDLLKIIKDK
DELDNEENEDILEDIVLTLTLFEDREMIEERLKTYAHLFDDKVMKQLKRRR
YTGWGRLSRKLINGIRDKQSGKTILDFLKSDGFANRNEMQLIHDDSLTFKE
DIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQN
EKLYLYYLQNGRDMYVDQELDINRLSDYDVDAIVPQSFLKDDSIDNKVLTR
SDKNRGKSDNVPSEEVVKKMKNYWRQLLNAKLITQRKEDNLTKAERGGLSE
LDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELLG
ITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLD
EIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDG
SPKKKRKVSS.

The nucleic acid encoding the NLS-dCas9-Clo051-NLS (“wildtype Cas-CLOVER”) fusion protein can be DNA or RNA. In some embodiments, a dCas9-Clo051 fusion protein comprising two NLS regions is encoded by an mRNA sequence comprising, consisting essentially of or consisting of SEQ ID NO: 29. In some embodiments, the dCas9-Clo051 fusion protein comprising two NLS regions is encoded by a polynucleotide comprising a sequence that is at least at least 65%, 70%, 75%, 80%, 85%, 90%, 95%, 96%, 97%, 98%, or 99% identical to the sequence set forth in SEQ ID NO: 29.

NLS-dCas9-Clo051-NLS mRNA sequence (NLS amino acid sequence is bolded and underlined):

(SEQ ID NO: 29)
atggcaccaaagaagaaaagaaaagtggagggcatcaagtcaaacatcagcctgctgaaagacg
aactgcggggacagattagtcacatcagtcacgagtacctgtcactgattgatctggccttcga
cagcaagcagaatagactgtttgagatgaaagtgctggaactgctggtcaacgagtatggcttc
aagggcagacatctgggcgggtctaggaaacctgacggcatcgtgtacagtaccacactggaag
acaacttcggaatcattgtcgataccaaggcttattccgagggctactctctgccaattagtca
ggcagatgagatggaaaggtacgtgcgcgaaaactcaaatagggacgaggaagtcaaccccaat
aagtggtgggagaatttcagcgaggaagtgaagaaatactacttcgtctttatctcaggcagct
tcaaagggaagtttgaggaacagctgcggagactgtccatgactaccggggtgaacggatctgc
tgtcaacgtggtcaatctgctgctgggcgcagaaaagatcaggtccggggagatgacaattgag
gaactggaacgcgccatgttcaacaattctgagtttatcctgaagtatggaggcgggggaagcg
ataagaaatactccatcggactggccattggcaccaattccgtgggctgggctgtcatcacaga
cgagtacaaggtgccaagcaagaagttcaaggtcctggggaacaccgatcgccacagtatcaag
aaaaatctgattggagccctgctgttcgactcaggcgagactgctgaagcaacccgactgaagc
ggactgctaggcgccgatatacccggagaaaaaatcggatctgctacctgcaggaaattttcag
caacgagatggccaaggtggacgatagtttctttcaccgcctggaggaatcattcctggtggag
gaagataagaaacacgagcggcatcccatctttggcaacattgtggacgaagtcgcttatcacg
agaagtaccctactatctatcatctgaggaagaaactggtggactccaccgataaggcagacct
gcgcctgatctatctggccctggctcacatgatcaagttccgggggcattttctgatcgaggga
gatctgaaccctgacaattctgatgtggacaagctgttcatccagctggtccagacatacaatc
agctgtttgaggaaaacccaattaatgcctcaggcgtggacgcaaaggccatcctgagcgccag
actgtccaaatctaggcgcctggaaaacctgatcgctcagctgccaggagagaagaaaaacggc
ctgtttgggaatctgattgcactgtccctgggcctgacacccaacttcaagtctaattttgatc
tggccgaggacgctaagctgcagctgtccaaagacacttatgacgatgacctggataacctgct
ggctcagatcggcgatcagtacgcagacctgttcctggccgctaagaatctgagtgacgccatc
ctgctgtcagatattctgcgcgtgaacacagagattactaaggccccactgagtgcttcaatga
tcaaaagatatgacgagcaccatcaggatctgaccctgctgaaggctctggtgaggcagcagct
gcccgagaaatacaaggaaatcttctttgatcagagcaagaatggatacgccggctatattgac
ggcggggcttcccaggaggagttctacaagttcatcaagcccattctggaaaagatggacggca
ccgaggaactgctggtgaagctgaatcgggaggacctgctgagaaaacagaggacatttgataa
cggaagcatccctcaccagattcatctgggcgaactgcacgccatcctgcgacggcaggaggac
ttctacccatttctgaaggataaccgcgagaaaatcgaaaagatcctgaccttcagaatcccct
actatgtggggcctctggcacggggaaatagtagatttgcctggatgacaagaaagtcagagga
aactatcaccccctggaacttcgaggaagtggtcgataaaggcgctagcgcacagtccttcatt
gaaaggatgacaaattttgacaagaacctgccaaatgagaaggtgctgcccaaacacagcctgc
tgtacgaatatttcacagtgtataacgagctgactaaagtgaagtacgtcaccgaagggatgcg
caagcccgcattcctgtccggagagcagaagaaagccatcgtggacctgctgtttaagacaaat
cggaaagtgactgtcaaacagctgaaggaagactatttcaagaaaattgagtgtttcgattcag
tggaaatcagcggcgtcgaggacaggtttaacgcctccctggggacctaccacgatctgctgaa
gatcatcaaggataaggacttcctggacaacgaggaaaatgaggacatcctggaggacattgtg
ctgacactgactctgtttgaggatcgcgaaatgatcgaggaacgactgaagacttatgcccatc
tgttcgatgacaaagtgatgaagcagctgaaaagaaggcgctacaccggatggggacgcctgag
ccgaaaactgatcaatgggattagagacaagcagagcggaaaaactatcctggactttctgaag
tccgatggcttcgccaacaggaacttcatgcagctgattcacgatgactctctgaccttcaagg
aggacatccagaaagcacaggtgtctggccagggggacagtctgcacgagcatatcgcaaacct
ggccggcagccccgccatcaagaaagggattctgcagaccgtgaaggtggtggacgaactggtc
aaggtcatgggacgacacaaacctgagaacatcgtgattgagatggcccgcgaaaatcagacaa
ctcagaagggccagaaaaacagtcgagaacggatgaagagaatcgaggaaggcatcaaggagct
ggggtcacagatcctgaaggagcatcctgtggaaaacactcagctgcagaatgagaaactgtat
ctgtactatctgcagaatggacgggatatgtacgtggaccaggagctggatattaacagactga
gtgattatgacgtggatgccatcgtccctcagagcttcctgaaggatgactccattgacaacaa
ggtgctgaccaggtccgacaagaaccgcggcaaatcagataatgtgccaagcgaggaagtggtc
aagaaaatgaagaactactggaggcagctgctgaatgccaagctgatcacacagcggaaatttg
ataacctgactaaggcagaaagaggaggcctgtctgagctggacaaggccggcttcatcaagcg
gcagctggtggagacaagacagatcactaagcacgtcgctcagattctggatagcagaatgaac
acaaagtacgatgaaaacgacaagctgatcagggaggtgaaagtcattactctgaaatccaagc
tggtgtctgactttagaaaggatttccagttttataaagtcagggagatcaacaactaccacca
tgctcatgacgcatacctgaacgcagtggtcgggaccgccctgattaagaaataccccaagctg
gagtccgagttcgtgtacggagactataaagtgtacgatgtccggaagatgatcgccaaatctg
agcaggaaattggcaaggccaccgctaagtatttcttttacagtaacatcatgaatttctttaa
gaccgaaatcacactggcaaatggggagatcagaaaaaggcctctgattgagaccaacggggag
acaggagaaatcgtgtgggacaagggaagggattttgctaccgtgcgcaaagtcctgtccatgc
cccaagtgaatattgtcaagaaaactgaagtgcagaccgggggattctctaaggagagtattct
gcctaagcgaaactctgataaactgatcgcccggaagaaagactgggaccccaagaagtatggc
gggttcgactctccaacagtggcttacagtgtcctggtggtcgcaaaggtggaaaaggggaagt
ccaagaaactgaagtctgtcaaagagctgctgggaatcactattatggaacgcagctccttcga
gaagaatcctatcgattttctggaagccaagggctataaagaggtgaagaaagacctgatcatt
aagctgccaaaatactcactgtttgagctggaaaacggacgaaagcgaatgctggcaagcgccg
gagaactgcagaagggcaatgagctggccctgccctccaaatacgtgaacttcctgtatctggc
tagccactacgagaaactgaaggggtcccctgaggataacgaacagaagcagctgtttgtggag
cagcacaaacattatctggacgagatcattgaacagatttcagagttcagcaagagagtgatcc
tggctgacgcaaatctggataaagtcctgagcgcatacaacaagcaccgagacaaaccaatccg
ggagcaggccgaaaatatcattcatctgttcaccctgacaaacctgggcgcccctgcagccttc
aagtattttgacaccacaatcgatcggaagagatacacttctaccaaagaggtgctggatgcta
ccctgatccaccagagtattaccggcctgtatgagacacgcatcgacctgtcacagctgggagg
cgatgggagccccaagaaaaagcggaaggtgtctagttaatga.

### Illustrative Mutant Cas-CLOVER Fusion Proteins

In some aspects, NLS-dCas9-Clo051-NLS (“wildtype Cas-CLOVER”) comprises at least one amino acid substitution. In some aspects, the amino acid substitution is located in the Clo051 domain of the NLS-dCas9-Clo051-NLS.

In some aspects, the NLS-dCas9-Clo051-NLS of SEQ ID NO: 28 can comprise at least one substitution at amino acid positions 42, 44, 67, 105, 107 and/or 153. In some aspects, the amino acid substitutions are F42E, F42D, S44E, S44P, R67E, I105Q, Q107A, Q107E, Q107H, Q107D and/or K153D. In some aspects, the amino acid substitution is S44P.

An exemplary S44P mutant NLS-dCas9-Clo051-NLS (“S44P Cas-CLOVER” or “S44P CC” or “S44P”) fusion protein can comprise, consist essentially of, or consist of, the amino acid sequence of SEQ ID NO: 35. In some embodiments, the S44P mutant NLS-dCas9-Clo051-NLS fusion protein comprises a sequence that is at least at least 65%, 70%, 75%, 80%, 85%, 90%, 95%, 96%, 97%, 98%, or 99% identical to the sequence set forth in SEQ ID NO: 35.

The S44P Cas-CLOVER fusion protein can be encoded by a polynucleotide which comprises, consists essentially of, or consists of, the nucleic acid sequence of SEQ ID NO: 36. In some embodiments, the S44P mutant NLS-dCas9-Clo051-NLS fusion protein is encoded by a polynucleotide comprising a sequence that is at least at least 65%, 70%, 75%, 80%, 85%, 90%, 95%, 96%, 97%, 98%, or 99% identical to the sequence set forth in SEQ ID NO: 36. The nucleic acid encoding the dCas9-Clo051 fusion protein can be DNA or RNA.

| S44P Cas-CLOVER amino acid sequence (SEQ ID NQ: 35): | | | | | | |
|---|---|---|---|---|---|---|
| MAPKKKRKVE | GIKSNISLLK | DELRGOISHI | SHEYLSLIDL | AFDPKONRLF | EMKVLELLVN | 60 |
| EYGFKGRHLG | GSRKPDGIVY | STTLEDNFGI | IVDTKAYSEG | YSLPISQADE | MERYVRENSN | 120 |
| RDEEVNPNKW | WENFSEEVKK | YYFVFISGSF | KGKFEEQLRR | LSMTTGVNGS | AVNVVNLLLG | 180 |
| AEKIRSGEMT | IEELERAMEN | NSEFILKYGG | GGSDKKYSIG | LAIGTNSVGW | AVITDEYKVP | 240 |
| SKKFKVLGNT | DRHSIKKNLI | GALLFDSGET | AEATRLKRTA | RRRYTRRKNR | ICYLQEIFSN | 300 |
| EMAKVDDSFF | HRLEESFLVE | EDKKHERHPI | FGNIVDEVAY | HEKYPTIYHL | RKKLVDSTDK | 360 |
| ADLRLIYLAL | AHMIKFRGHF | LIEGDLNPDN | SDVDKLFIQL | VQTYNQLFEE | NPINASGVDA | 420 |
| KAILSARLSK | SRRLENLIAQ | LPGEKKNGLF | GNLIALSLGL | TPNFKSNEDL | AEDAKLQLSK | 480 |
| DTYDDDLDNL | LAQIGDQYAD | LFLAAKNLSD | AILLSDILRV | NTEITKAPLS | ASMIKRYDEH | 540 |
| HQDLTLLKAL | VRQQLPEKYK | EIFFDQSKNG | YAGYIDGGAS | QEEFYKFIKP | ILEKMDGTEE | 600 |
| LLVKLNREDL | LRKQRTFDNG | SIPHQIHLGE | LHAILRRQED | FYPFLKDNRE | KIEKILTFRI | 660 |
| PYYVGPLARG | NSRFAWMTRK | SEETITPWNF | EEVVDKGASA | QSFIERMTNF | DKNLPNEKVL | 720 |
| PKHSLLYEYF | TVYNELTKVK | YVTEGMRKPA | FLSGEQKKAI | VDLLFKTNRK | VTVKQLKEDY | 780 |
| FKKIECFDSV | EISGVEDREN | ASLGTYHDLL | KIIKDKDELD | NEENEDILED | IVLTLTLFED | 840 |
| REMIEERLKT | YAHLEDDKVM | KQLKRRRYTG | WGRLSRKLIN | GIRDKQSGKT | ILDFLKSDGF | 900 |
| ANRNFMQLIH | DDSLTFKEDI | QKAQVSGQGD | SLHEHIANLA | GSPAIKKGIL | QTVKVVDELV | 960 |
| KVMGRHKPEN | IVIEMARENQ | TTQKGQKNSR | ERMKRIEEGI | KELGSQILKE | HPVENTQLQN | 1020 |
| EKLYLYYLQN | GRDMYVDQEL | DINRLSDYDV | DAIVPQSFLK | DDSIDNKVLT | RSDKNRGKSD | 1080 |
| NVPSEEVVKK | MKNYWRQLLN | AKLITQRKFD | NLTKAERGGL | SELDKAGFIK | RQLVETRQIT | 1140 |
| KHVAQILDSR | MNTKYDENDK | LIREVKVITL | KSKLVSDERK | DFQFYKVREI | NNYHHAHDAY | 1200 |
| LNAVVGTALI | KKYPKLESEF | VYGDYKVYDV | RKMIAKSEQE | IGKATAKYFF | YSNIMNFFKT | 1260 |
| EITLANGEIR | KRPLIETNGE | TGEIVWDKGR | DFATVRKVLS | MPQVNIVKKT | EVQTGGFSKE | 1320 |
| SILPKRNSDK | LIARKKDWDP | KKYGGFDSPT | VAYSVLVVAK | VEKGKSKKLK | SVKELLGITI | 1380 |
| MERSSFEKNP | IDFLEAKGYK | EVKKDLIIKL | PKYSLFELEN | GRKRMLASAG | ELQKGNELAL | 1440 |
| PSKYVNFLYL | ASHYEKLKGS | PEDNEQKQLF | VEQHKHYLDE | IIEQISEFSK | RVILADANLD | 1500 |
| KVLSAYNKHR | DKPIREQAEN | IIHLFTLTNL | GAPAAFKYFD | TTIDRKRYTS | TKEVLDATLI | 1560 |
| HQSITGLYET | RIDLSQLGGD | GSPKKKRKVS | S | | | 1591 |
| S44P Cas-CLOVER nucleic acid sequence (SEQ ID NO: 36) | | | | | | |
| atggctccca | agaagaagcg | gaaggtcgag | ggcatcaaga | gcaacatcag | cctgctgaag | 60 |
| gacgagctga | gaggccagat | cagccacatc | tcccacgagt | acctgagcct | gatcgacctg | 120 |
| gccttcgacc | ccaagcagaa | ccggctgttc | gagatgaagg | tgctggaact | gctggtcaac | 180 |
| gagtacggct | tcaagggcag | acacctcgg | ggcagcagaa | agcctgatgg | catcgtgtac | 240 |
| agcaccacac | tcgaggacaa | cttcggcatc | atcgtggaca | ccaaggccta | cagcgagggc | 300 |
| tacagcctgc | ctatctctca | ggccgacgag | atggaaagat | acgtgcgcga | gaacagcaac | 360 |
| cgcgacgagg | aagtgaaccc | caacaagtgg | tgggagaact | tcagcgagga | agtcaaaaag | 420 |
| tactacttcg | tgttcatcag | cggcagcttt | aagggcaagt | tcgaggaaca | gctgcggcgg | 480 |
| ctgtctatga | ccacaggcgt | taacggcage | gccgtgaacg | tggtcaatct | gctgctgggc | 540 |
| gccgagaaga | ttagaagcgg | cgagatgacc | atcgaggaac | tggaacgggc | catgttcaac | 600 |
| aacagcgagt | tcatcctgaa | gtacggcgga | ggcggcagcg | acaagaagta | ctctatcgga | 660 |
| ctggccatcg | gcaccaactc | tgttggatgg | gccgtgatca | ccgacgagta | caaggtgccc | 720 |
| agcaagaaat | tcaaagtgct | gggcaacacc | gaccggcaca | gcatcaagaa | gaatctgatc | 780 |
| ggcgccctgc | tgttcgactc | tggcgaaaca | gccgaagcca | ccagactgaa | gagaaccgcc | 840 |
| agacggcggt | acaccagaag | aaagaaccgg | atctgctacc | tgcaagagat | cttcagcaac | 900 |
| gagatggcca | aggtggacga | cagottottc | cacagactgg | aagagtcctt | cctggtggaa | 960 |
| gaggacaaga | agcacgagcg | gcaccccatc | ttcggaaata | tcgtggacga | ggtggcctac | 1020 |
| cacgagaagt | accccaccat | ctaccacctg | agaaagaaac | tggtggacag | caccgacaag | 1080 |
| gccgacctgc | gactgatcta | tctggccctg | gctcacatga | tcaagttccg | gggccacttc | 1140 |
| ctgatcgagg | gcgacctgaa | tcctgacaac | tccgacgtgg | acaagctgtt | catccagctg | 1200 |
| gtgcagacct | acaatcagct | gttcgaagag | aatcccatca | acgcctctgg | cgtggacgcc | 1260 |
| aaagccatcc | tgtctgccag | actgagcaag | agcagacggc | tggaaaacct | gatcgctcag | 1320 |
| ctgcccggcg | agaagaagaa | tggcctgttc | ggcaacctga | ttgccctgtc | tctgggcctg | 1380 |
| acacctaact | tcaagtccaa | cttcgatctg | gccgaggatg | ccaaactgca | gctgtccaag | 1440 |
| gacacctacg | acgacgacct | ggataacctg | ctggcccaga | tcggcgatca | gtacgccgac | 1500 |
| ttgtttctgg | ccgccaagaa | cctgtctgac | gccatcctgc | tgagcgacat | cctgagagtg | 1560 |
| aacaccgaga | tcacaaaggc | ccctctgagc | gcctctatga | tcaagagata | cgacgagcac | 1620 |
| caccaggatc | tgaccctgct | gaaagctctc | gtcaggcagc | agctgccaga | gaagtacaaa | 1680 |
| gagattttct | tcgaccagag | caagaacggc | tacgccggct | acattgatgg | cggagccagc | 1740 |
| caagaggaat | tctacaagtt | catcaagccc | atcctcgaga | agatggacgg | cacagaggaa | 1800 |
| ctgctcgtga | agctgaacag | agaggacctg | ctgcggaagc | agcggacctt | cgacaatggc | 1860 |
| tctatccctc | accagatcca | cctgggagag | ctgcacgcca | ttctgcggag | acaagaggac | 1920 |
| ttttacccat | tectgaagga | caaccgggaa | aagattgaga | agatcctgac | cttcaggatc | 1980 |
| ccctactacg | tgggaccact | ggccagaggc | aatagcagat | tcgcctggat | gaccagaaag | 2040 |
| agcgaggaaa | ccatcacacc | ctggaacttc | gaagaggtgg | tggacaaggg | cgccagcgct | 2100 |
| cagtccttca | tcgagcggat | gaccaatttc | gacaagaatc | tgcccaacga | gaaagtgctg | 2160 |
| cccaagcact | ccctgctgta | cgagtacttc | accgtgtaca | acgagctgac | caaagtgaaa | 2220 |
| tacgtgaccg | agggaatgag | aaagcccgcc | tttctgtccg | gcgagcagaa | aaaggccatc | 2280 |
| gtggatctgc | tgttcaagac | caaccggaaa | gtgaccgtga | agcagctgaa | agaggactac | 2340 |
| ttcaagaaaa | tcgagtgctt | cgactccgtg | gaaatcagcg | gcgtggaaga | tcggttcaat | 2400 |
| gccagcctgg | gcacatacca | cgatctgctg | aaaattatca | aggacaagga | cttcctggac | 2460 |
| aacgaggaaa | acgaggacat | ccttgaggac | atcgtgctga | ccctgacact | gttcgaggac | 2520 |
| agagagatga | tcgaggaaag | gctgaaaaca | tacgcccacc | tgttcgacga | caaagtcatg | 2580 |
| aagcaactga | agcggggcg | ctacacaggc | tggggcagac | tgtctagaaa | gctgatcaac | 2640 |
| ggcatccggg | acaagcagtc | cggcaagacc | atcctggact | ttctgaagtc | cgacggcttc | 2700 |
| gccaacagaa | acttcatgca | gctgattcac | gacgacagcc | tcaccttcaa | agaggacatt | 2760 |
| cagaaggccc | aggtttccgg | ccagggcgat | tctctgcacg | agcacattgc | caatctggcc | 2820 |
| ggctctcccg | ccattaagaa | gggcattctg | cagacagtga | aagtggtgga | tgagctggtc | 2880 |
| aaagtgatgg | ggagacacaa | gcccgagaac | atcgtgatcg | aaatggccag | agagaaccag | 2940 |
| accacacaga | agggccagaa | gaactcccgc | gagagaatga | agcggatcga | agagggaatc | 3000 |
| aaagagctgg | ggagccagat | cctgaaagaa | caccccgtgg | aaaacaccca | gctgcagaac | 3060 |
| gagaagctgt | acctgtacta | cctccagaac | ggccgggata | tgtacgtgga | ccaagagctg | 3120 |
| gacatcaacc | gcctgagcga | ctacgatgtg | gacgctatcg | tgccccagtc | ttttctgaaa | 3180 |
| gatgactcca | tcgacaacaa | ggtgctgacc | agaagcgata | agaaccgggg | caagagcgac | 3240 |
| aacgtgccct | ctgaagaggt | cgtgaagaag | atgaagaact | actggcgaca | gctgctgaac | 3300 |
| gccaagctga | ttacccagcg | gaagttcgat | aacctgacca | aggccgagag | aggcggcctg | 3360 |
| tctgaactgg | ataaggccgg | cttcatcaag | agacagctgg | tggaaacccg | gcagatcacc | 3420 |
| aaacacgtgg | cacagattct | ggactcccgg | atgaacacca | aatacgatga | gaacgacaaa | 3480 |
| ctgatccggg | aagtgaaagt | catcaccctg | aagtccaagc | tggtgtccga | tttccggaag | 3540 |
| gatttccagt | tctacaaagt | gcgggaaatc | aacaactacc | atcacgccca | cgacgcctac | 3600 |
| ctgaatgccg | ttgttggaac | agccctgatc | aagaagtatc | ccaagctgga | aagcgagttc | 3660 |
| gtgtacggcg | actacaaggt | gtacgacgtg | cggaagatga | tcgccaagag | cgagcaagag | 3720 |
| attggaaagg | ctaccgccaa | atacttcttc | tactccaaca | tcatgaactt | tttcaagaca | 3780 |
| gagatcaccc | tcgccaacgg | cgagatcaga | aagcggcctc | tgatcgagac | aaacggcgaa | 3840 |
| accggcgaga | ttgtgtggga | taagggcaga | gactttgcca | cagtgcggaa | ggtgctcagc | 3900 |
| atgccccaag | tgaatatcgt | gaaaaagacc | gaggtgcaga | caggcggctt | cagcaaagag | 3960 |
| tccattctgc | ctaagcggaa | ctccgacaag | ctgatcgccc | ggaagaagga | ctgggacccc | 4020 |
| aagaaatacg | gcggcttcga | tagccctacc | gtggcctatt | ctgtgctggt | ggtggccaaa | 4080 |
| gtggaaaagg | gaaagtccaa | gaagctcaag | agcgtcaaag | aactcctggg | catcaccatc | 4140 |
| atggaacggt | ccagcttcga | gaagaaccct | atcgactttc | tggaagccaa | gggctacaaa | 4200 |
| gaagtcaaga | aggacctgat | catcaagctc | cccaagtaca | gcctgttcga | gctggaaaat | 4260 |
| ggccggaagc | ggatgctggc | ttctgctggc | gaactgcaga | agggaaacga | actggccctg | 4320 |
| cctagcaaat | atgtgaactt | cctgtacctg | gccagccact | atgagaagct | gaagggcagc | 4380 |
| cccgaggaca | atgagcagaa | gcagcttttc | gtcgagcage | acaagcacta | cctggacgag | 4440 |
| atcatcgagc | agatctccga | gttctccaag | agagtgatcc | tggccgacgc | caacctggac | 4500 |
| aaggttctgt | ccgcctacaa | caagcaccgg | gataagccca | tcagagagca | ggccgagaat | 4560 |
| atcatccacc | tgtttaccct | gaccaacctg | ggagcccctg | ccgccttcaa | gtacttcgac | 4620 |
| accaccatcg | accggaagcg | ctacaccagc | accaaagaag | tgctggacgc | cacactgatc | 4680 |
| caccagagca | tcaccggcct | gtacgagaca | cggatcgatc | tgtctcagct | tggaggcgac | 4740 |
| ggcagcccta | agaagaagag | aaaggtttcc | agctaataa | | | 4779 |

### Transgenes

In some aspects, the compositions described herein may be used to deliver a transgene to a cell. A cell comprising the gene editing composition can express the gene editing composition stably or transiently.

The transgene can comprise a sequence encoding a therapeutic agent. The therapeutic agent can be a protein or an RNA that provides a therapeutic benefit when administered to a cell or a subject. The therapeutic agent can be a therapeutic protein or a therapeutic RNA. The therapeutic agent can be human beta-globin (HBB), T87Q human beta-globin (HBB T87Q), BAF chromatin remodeling complex subunit (BCL11A) shRNA, insulin like growth factor 2 binding protein 1 (IGF2BP1), interleukin 2 receptor gamma (IL2RG), alpha galactosidase A (GLA), alpha-L-idurondase (IDUA), iduronate 2-sulfatase (IDS), cystinosin lysosomal cysteine transporter (CTNS). The transgene can comprise a sequence of Factor VIII or Factor IX. The transgene can comprise a sequence encoding a chimeric antigen receptor (CAR). The transgene can comprise a sequence encoding a non-naturally occurring chimeric stimulatory receptor (CSR) comprising: (a) an ectodomain comprising a activation component, wherein the activation component is isolated or derived from a first protein; (b) a transmembrane domain; and (c) an endodomain comprising at least one signal transduction domain, wherein the at least one signal transduction domain is isolated or derived from a second protein; wherein the first protein and the second protein are not identical. In one aspect, the transgene can comprise a sequence for a CAR and a sequence for a CSR. In one aspect, the transgene comprising a CAR or a CSR specifically binds to BCMA, PSMA, MUC1-C, CD133, c-KIT, CD19 or CD20. The transgene can comprise a sequence encoding for an inducible proapoptotic polypeptide comprising (a) a ligand binding region, (b) a linker, and (c) a caspase polypeptide, wherein the inducible proapoptotic polypeptide does not comprise a non-human sequence. The transgene can be integrated into the genome of the HSC. The integration can be stable or transient.

Factor VIII (FVIII) deficiency leads to development of Hemophilia A. Factor IX (FIX) deficiency leads to development of Hemophilia B. Prior to the compositions and methods of the disclosure, the standard treatment for hemophilia B involved an infusion of recombinant FIX every 2 to 3 days, at an expense of approximately $250,000 per year. In sharp contrast to this standard treatment option, iPSCs of the disclosure can be differentiated into any cell type including HSCs and maintained in humans for several decades.

gRNAs

In some aspects, a guide RNA is used to deliver the transgenes provided herein to the target cell.

The guide RNA can comprise a sequence complementary to a target sequence within a genomic DNA sequence. The target sequence within a genomic DNA sequence can be a target sequence within a safe harbor site of a genomic DNA sequence. Exemplary target sequences include but are not limited to ALB, HBB, TRAC, B2M, TCRb, GAPDH or SOX17.

The guide RNA can comprise a sequence complementary to at least one target sequence on a transposon, plasmid or vector. In some aspects, the complementary sequence to the guide RNA on the transposon, plasmid or vector is located within the transgene for targeted nucleic acid insertion. In some aspects, the complementary sequence to the guide RNA on the transposon, plasmid or vector is located within the transgene for targeted nucleic acid insertion. In some aspects, the complementary sequence on the transposon, plasmid or vector facilitates binding of a gRNA which is bound to an effector molecule, thereby tethering all components. In some aspects, the effector molecule is Cas-CLOVER. In some aspects, the Cas-CLOVER further comprises at least one NLS sequence. In some aspects, the NLS sequence of the Cas-CLOVER facilitates localization of the tethered components to the nucleus. This promotes localization of all components required for gene editing into the nucleus (Cas-CLOVER, gRNA and transposon, plasmid or vector), thereby increasing efficiency of gene editing.

As used herein, the term “guide sequence” in the context of a Cas-CLOVER system or a CRISPR-Cas9 system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequence may form a duplex with a target sequence. The duplex may be a DNA duplex, an RNA duplex, or a RNA/DNA duplex. The terms “guide molecule” and “guide RNA” and “single guide RNA” are used interchangeably herein to refer to RNA-based molecules that are capable of forming a complex with a Cas-CLOVER or a CRISPR-Cas protein and comprises a guide sequence having sufficient complementarity with a target nucleic acid sequence to hybridize with the target nucleic acid sequence and direct sequence-specific binding of the complex to the target nucleic acid sequence. The guide molecule or guide RNA may encompass RNA-based molecules having one or more chemically modifications (e.g., by chemical linking two ribonucleotides or by replacement of one or more ribonucleotides with one or more deoxyribonucleotides), as described herein.

The term “target region”, “target sequence” or “protospacer” as used interchangeably herein refers to the region of the target gene to which the Cas-Clover system or the CRISPR/Cas9-based system targets. The Cas-CLOVER or the CRISPR/Cas9-based system may include at least one gRNA, wherein the gRNAs target different DNA sequences. The target DNA sequences may be overlapping. The Cas-CLOVER system may include at least two gRNAs, wherein the gRNAs target different DNA sequences. The target sequence or protospacer is followed by a PAM sequence at the 3′ end of the protospacer. Different Type II systems have differing PAM requirements. For example, the S. pyogenes Type II system uses an “NGG” sequence, where “N” can be any nucleotide.

The guide RNA or the guide RNA of a Cas-Clover protein or a CRISPR-Cas protein may comprise a tracr-mate sequence (encompassing a “direct repeat” in the context of an endogenous CRISPR system) and a guide sequence (also referred to as a “spacer” in the context of an endogenous CRISPR system). In some embodiments, the Cas-CLOVER or the CRISPR-Cas system or complex as described herein does not comprise and/or does not rely on the presence of a tracr sequence. In certain embodiments, the guide molecule may comprise, consist essentially of, or consist of a direct repeat sequence fused or linked to a guide sequence or spacer sequence.

In certain embodiments, the guide sequence or spacer length of the guide molecules is 15 to 50 nucleotides in length. In certain embodiments, the spacer length of the guide RNA is at least 15 nucleotides in length. In certain embodiments, the spacer length is from 15 to 17 nucleotides in length, from 17 to 20 nucleotides in length, from 20 to 24 nucleotides in length, from 23 to 25 nucleotides in length, from 24 to 27 nucleotides in length, from 27-30 nucleotides in length, from 30-35 nucleotides in length, or greater than 35 nucleotides in length.

In some embodiments, the guide sequence is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and PA Carr and GM Church, 2009, Nature Biotechnology 27 (12): 1151-62).

As described above, the Cas-Clover system and the CRISPR/Cas9 system utilize targeting gRNA and a shuttling gRNA that provides the targeting of the Cas-Clover system and the CRISPR/Cas9-based system. The gRNA may be a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to cleave the target nucleic acid.

In some embodiment, the gRNA is a left gRNA and targets a region upstream of the target gene cut site (e.g., Intron 3 of the albumin locus), e.g., between 0-1000 bp upstream of a target gene. In some embodiments, the gRNA targets a region between 0-50 bp, 0-100 bp, 0-150 bp, 0-200 bp, 0-250 bp, 0-300 bp, 0-350 bp, 0-400 bp, 0-450 bp, 0-500 bp, 0-550 bp, 0-600 bp, 0-650 bp, 0-700 bp, 0-750 bp, 0-800 bp, 0-850 bp, 0-900 bp, 0-950 bp or 0-1000 bp upstream of the transcription start site of the target gene. In some embodiments, the gRNA targets a region within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the target gene.

In some embodiments, the gRNA is a right gRNA and targets a region downstream of a target gene cut site e.g., between 0-1000 bp downstream of a target gene. In some embodiments, the gRNA targets a region between 0-50 bp, 0-100 bp, 0-150 bp, 0-200 bp, 0-250 bp, 0-300 bp, 0-350 bp, 0-400 bp, 0-450 bp, 0-500 bp, 0-550 bp, 0-600 bp, 0-650 bp, 0-700 bp, 0-750 bp, 0-800 bp, 0-850 bp, 0-900 bp, 0-950 bp or 0-1000 bp downstream of the target gene. In some embodiments, the gRNA targets a region within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp downstream of the target gene.

In some embodiments, the left gRNA and right gRNA of the gRNA pair are designed with the proper spacing to allow for dimerization of the Clo051 nuclease domains upon binding of the left and right gRNAs to their target sequence of the target region. In some embodiments, the first and second gRNA sequences are complementary to the first and the second target sequences of the AAV donor polynucleotide, respectively, and are designed of proper spacing to allow for targeting Cas-CLOVER to the AAV donor polynucleotide to cleave the AAV donor polynucleotide thereby linearizing the polynucleotide and removing the terminal AAV ITR sequences.

gRNA can be divided into a target binding region and a Cas9 binding region. The target binding region hybridizes with a target region in a target gene. Methods for designing such target binding regions are known in the art, see, e.g., Doench et al., Nat Biotechnol. (2014) 32:1262-7; and Doench et al., Nat Biotechnol. (2016) 34:184-91, incorporated by reference herein in their entirety. Design tools are available at, e.g., Feng Zhang lab's target Finder, Michael Boutros lab's Target Finder (E-CRISP), RGEN Tools (Cas-OF Finder), CasFinder, and CRISPR Optimal Target Finder. In certain embodiments, the target binding region can be between about 15 and about 50 nucleotides in length (about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 nucleotides in length). In certain embodiments, the target binding region can be between about 19 and about 21 nucleotides in length. In one embodiment, the target binding region is 15, 16, 17, 18, 19, 20, 21, 22, 23, 24, or 25 nucleotides in length.

In one embodiment, the target binding region is complementary, e.g., completely complementary, to the target region in the target gene. In one embodiment, the target binding region is substantially complementary to the target region in the target gene. In one embodiment, the target binding region comprises no more than 1, 2, 3, 4, 5, 6, 7, 8, 9, or 10 nucleotides that are not complementary to the target region in the target gene.

Exemplary sgRNAs of the disclosure include but are not limited to sequences for targeting intron 3 of the albumin gene. Exemplary sgRNAs of the disclosure comprise, consist essentially of or consists of the sequences as shown in Table 1.

TABLE 1
Exemplary sgRNAs of the disclosure

| | | SEQ ID |
|---|---|---|
| Exemplary sgRNA | Sequence | NO: |
| Left Alb intron 3 | GATGACCATTGGTATTGGAG | SEQ ID |
| sgRNA | | NO: 4 |
| Right Alb intron 3 | GAATAACATGCAGACTAAGC | SEQ ID |
| sgRNA | | NO: 5 |

### Vectors of the Present Disclosure

The present disclosure provides compositions comprising a vector, wherein the vector comprises at least one adeno-associated virus (AAV) donor polynucleotide. A vector comprising at least one adeno-associated virus (AAV) donor polynucleotide is herein referred to as an “AAV donor vector”.

A vector of the present disclose can be a viral vector or a recombinant vector. Viral vectors can comprise a sequence isolated or derived from a retrovirus, a lentivirus, an adenovirus, an adeno-associated virus or any combination thereof. The viral vector may comprise a sequence isolated or derived from an adeno-associated virus (AAV). The viral vector may comprise a recombinant AAV (rAAV).

Exemplary adeno-associated viruses and recombinant adeno-associated viruses include, but are not limited to all serotypes (e.g., AAV1, AAV2, AAV3, AAV4, AAV5, AAV6, AAV7, AAV8, AAV9, AAV10 and AAV11). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, AAV8, AAV9, rAAV-LK03, AAV-KP-1 (also referred to as AAV-KP1; described in detail in Kerun et al. JCI Insight, 2019; 4 (22): el31610) and AAV-NP59 (described in detail in Paulk et al. Molecular Therapy, 2018; 26 (1): 289-303).

The present disclosure provides a composition comprising a plurality of AAV-KP-1 particles comprising at least one adeno-associated virus (AAV) donor polynucleotide. The present disclosure provides a composition comprising a plurality of AAV-NP59 particles comprising at least one adeno-associated virus (AAV) donor polynucleotide.

The present disclosure provides a composition comprising a plurality of AAV-NP59 (described in detail in Paulk et al. Molecular Therapy, 2018; 26 (1): 289-303).

The present disclosure provides a composition comprising a plurality of AAV8 particles comprising at least one adeno-associated virus (AAV) donor polynucleotide and a LNP composition comprising a targeting gRNA pair and an mRNA encoding a Cas-CLOVER. In certain aspects, the mRNA molecule further comprises a 5′-CAP. In certain aspects, the CAS-CLOVER is a wild type Cas-CLOVER sequence, a S44P mutant Cas-CLOVER sequence or other Cas-CLOVER sequence described herein. In certain aspects, the at least one LNP composition comprises: about 54% of ssPalmO-Ph-P4C2 by moles, about 35% of cholesterol by moles, about 5% of DOPC by moles, about 5% DSPC by moles, and about 1% of DMG-PEG2000 by moles.

The present disclosure provides a composition comprising a plurality of AAV9 particles comprising at least one adeno-associated virus (AAV) donor polynucleotide and a LNP composition comprising a targeting gRNA pair and an mRNA encoding a Cas-CLOVER. In certain aspects, the mRNA molecule further comprises a 5′-CAP. In certain aspects, the CAS-CLOVER is a wild type Cas-CLOVER sequence, a S44P mutant Cas-CLOVER sequence or other Cas-CLOVER sequence described herein. In certain aspects, the at least one LNP composition comprises: about 54% of ssPalmO-Ph-P4C2 by moles, about 35% of cholesterol by moles, about 5% of DOPC by moles, about 5% DSPC by moles, and about 1% of DMG-PEG2000 by moles.

The viral vectors and viral particles of the present disclosure can be produced using standard methods known in the art.

The cell delivery compositions (e.g., polynucleotides, vectors) disclosed herein can comprise a nucleic acid encoding a therapeutic protein or therapeutic agent. Examples of therapeutic proteins include those disclosed in PCT Publication No. WO 2019/173636 and WO/2020/051374, each of which is incorporated herein by reference in its entirety for examples of therapeutic proteins that may be delivered using the compositions described herein. Therapeutic proteins can also include, but are not limited to, any one of polypeptides described herein as part of transgene sequences (e.g. hPAH).

### Formulations, Dosages and Modes of Administration

The present disclosure provides formulations, dosages and methods for administration of the compositions described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998.

Pharmaceutically acceptable carriers can be routinely selected that are suitable for the mode of administration, solubility and/or stability of the compositions as well known in the art or as described herein.

For example, the disclosed LNP compositions of the present disclosure can further comprise a diluent. In some compositions, the diluent can be phosphate buffered saline (“PBS”). In some compositions, the diluent can be sodium acetate.

Non-limiting examples of pharmaceutical excipients and additives suitable for use include proteins, peptides, amino acids, lipids, and carbohydrates (e.g. sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (EISA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffmose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffmose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-P-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g, EDTA).

Many known and developed modes can be used for administering therapeutically effective amounts of the compositions or pharmaceutical compositions disclosed herein. Non limiting examples of modes of administration include bolus, buccal, infusion, intraarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

A composition of the disclosure can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994,), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

For parenteral administration, any composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or triglycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

Formulations for oral administration rely on the co-administration of adjuvants (e.g., resorcinols and nonionic surfactants, such as polyoxyethylene oleyl ether and n-hexadecylpoly ethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. Formulations for delivery of hydrophilic agents including proteins and protein scaffolds and a combination of at least two surfactants intended for oral, buccal, mucosal, nasal, pulmonary, vaginal transmembrane, or rectal administration are described in U.S. Pat. No. 6,309,663. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant, such as magnesium stearate, paraben, preserving agent, such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. Nos. 5,879,681 and 5,871,753 and used to deliver biologically active agents orally are known in the art.

For pulmonary administration, preferably, a composition or pharmaceutical composition described herein is delivered in a particle size effective for reaching the lower airways of the lung or sinuses. The composition or pharmaceutical composition can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers (e.g, jet nebulizer, ultrasonic nebulizer), dry powder generators, sprayers, and the like. All such devices can use formulations suitable for the administration for the dispensing of a composition or pharmaceutical composition described herein in an aerosol. Such aerosols can be comprised of either solutions (both aqueous and non-aqueous) or solid particles. Additionally, a spray including a composition or pharmaceutical composition described herein can be produced by forcing a suspension or solution of at least one protein scaffold through a nozzle under pressure. In a metered dose inhaler (MDI), a propellant, a composition or pharmaceutical composition described herein, and any excipients or other additives are contained in a canister as a mixture including a liquefied compressed gas. Actuation of the metering valve releases the mixture as an aerosol, preferably containing particles in the size range of less than about 10 μm, preferably, about 1 μm to about 5 μm, and, most preferably, about 2 μm to about 3 μm. A more detailed description of pulmonary administration, formulations and related devices is disclosed in PCT Publication No. WO 2019/049816.

For absorption through mucosal surfaces, compositions include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the disclosure can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695). A more detailed description of mucosal administration and formulations is disclosed in PCT Publication No. WO 2019/049816.

For transdermal administration, a composition or pharmaceutical composition disclosed herein is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599). A more detailed description of transdermal administration, formulations and suitable devices is disclosed in PCT Publication No. WO 2019/049816.

It can be desirable to deliver the disclosed compounds to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the compositions or pharmaceutical compositions disclosed herein is from about 1 mg/kg, up to about 3, about 6 or about 12 mg/kg of body weight of the subject.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably, 0.1 to 10 milligrams per kilogram per administration or in sustained release form is effective to obtain desired results.

As a non-limiting example, treatment of humans or animals can be provided as a one time or periodic dosage of the compositions or pharmaceutical compositions disclosed herein about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

Dosage forms suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

An effective amount can comprise an amount of about 0.001 to about 500 mg/kg per single (e.g, bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

In aspects where the compositions to be administered to a subject in need thereof are modified cells as disclosed herein, the cells can be administered between about 1×10³ and 1×10¹⁵ cells; 1×10³ and 1×10¹⁵ cells, about 1×10⁴ and 1×10¹² cells; about 1×10⁵ and 1×10¹⁰ cells; about 1×10⁶ and 1×10⁹ cells; about 1×10⁶ and 1×10⁸ cells; about 1×10⁶ and 1×10⁷ cells; or about 1×10⁶ and 25×10⁶ cells. In an aspect the cells are administered between about 5×10⁶ and 25×10⁶ cells.

A detailed description of pharmaceutically acceptable excipients, formulations, dosages and methods of administration of the disclosed compositions and pharmaceutical compositions is disclosed in PCT Publication No. WO 2019/049816 which is incorporated herein by reference in its entirety.

Any use or method of the present disclosure can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one chemotherapeutic agent (e.g., an alkylating agent, a mitotic inhibitor, or a radiopharmaceutical).

In some aspects, the subject does not develop graft vs. host (GvH) and/or host vs. graft (HvG) following administration. In an aspect, the administration is systemic. Systemic administration can be any means known in the art and described in detail herein. Preferably, systemic administration is by an intravenous injection or an intravenous infusion. In an aspect, the administration is local. Local administration can be any means known in the art and described in detail herein. Preferably, local administration is by intra-tumoral injection or infusion, intraspinal injection or infusion, intracerebroventricular injection or infusion, intraocular injection or infusion, or intraosseous injection or infusion.

In some aspects, the therapeutically effective dose is a single dose. In some aspects, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously. In some aspects, where the composition is autologous cells or allogeneic cells, the dose is an amount sufficient for the cells to engraft and/or persist for a sufficient time to treat the disease or disorder.

### Methods of Use

The present disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human. The terms “subject” and “patient” are used interchangeably herein.

The disclosure provides a method of treating phenylketonuria (PKU) in a subject in need thereof comprising administering to the subject a) at least one therapeutically effective dose of a composition comprising the polynucleotide, vector or pharmaceutical composition of the present disclosure; and b) at least one LNP composition comprising a targeting gRNA pair and an mRNA encoding a Cas-CLOVER. In certain aspects, the mRNA molecule further comprises a 5′-CAP. In certain aspects, the CAS-CLOVER is a wild type Cas-CLOVER sequence, a S44P mutant Cas-CLOVER sequence or other Cas-CLOVER sequence described herein. In certain aspects, the at least one LNP composition comprises: about 54% of ssPalmO-Ph-P4C2 by moles, about 35% of cholesterol by moles, about 5% of DOPC by moles, about 5% DOPE by moles, and about 1% of DMG-PEG2000 by moles.

The present disclosure provides at least one composition of the present disclosure for the use in the treatment of PKU in a subject, wherein the at least one composition is for administration to the subject in at least one therapeutically effective amount.

The present disclosure provides the use of at least one composition of the present disclosure for the manufacture of a medicament for the treatment of PKU in a subject, wherein the at least one composition is for administration to the subject in at least one therapeutically effective amount.

The present disclosure provides a method of treating at least one disease in a subject, the method comprising administering to the subject at least one therapeutically effective amount of at least one composition of the present disclosure comprising at least one nucleic acid encoding a therapeutic protein.

In some aspects, a nucleic acid molecule formulated in a composition of the present disclosure can comprise at least one transgene sequence. In some aspects, a transgene sequence can comprise a nucleotide sequence encoding at least one therapeutic protein.

In some aspects, a nucleic acid molecule formulated in a composition of the present disclosure comprising AAV donor polynucleotides can comprise at least one transgene sequence. In some aspects, a transgene sequence can comprise a nucleotide sequence encoding at least one therapeutic protein.

In some aspects, an at least one sequence encoding at least one therapeutic protein can be a sequence encoding a human phenylalanine hydroxylase (hPAH) polypeptide, wherein the hPAH polypeptide comprises the nucleic acid sequence of SEQ ID NO: 9. In certain aspects, the nucleotide sequence encoding hPAH is codon optimized.

In some aspects, a hPAH polypeptide comprises, consists essentially of or consist of a nucleic acid sequence at least 65%, 70%, 75%, 80%, 85%, 90%, 95%, 96%, 97%, 98%, or 99% (or any percentage in between) identical to SEQ ID NO: 9. In some aspects, a hPAH polypeptide comprises, consists essentially of or consist of the nucleic acid sequence set forth in SEQ ID NO: 9.

In some aspects of the preceding methods, a composition comprising a nucleic acid molecule comprising a nucleotide sequence encoding at least one gRNA pair and a at least one mRNA encoding Cas-CLOVER can be a composition comprising at least one LNP of the present disclosure, wherein the LNP comprises at least one nucleic acid molecule comprising a nucleotide sequence encoding at least one Cas-CLOVER. Accordingly, the present disclosure provides methods of treating at least one disease in a subject, the methods comprising administering to the subject: a) at least one therapeutically effective amount of a composition comprising a AAV donor polynucleotide, wherein the polynucleotide comprises a nucleotide sequence encoding at least one therapeutic protein; and b) at least one therapeutically effective amount of LNPs of the present disclosure, wherein the LNPs comprise at least one targeting gRNA pair and at least one nucleic acid comprising a nucleotide sequence encoding at least one Cas-CLOVER.

In some aspects of the preceding methods, a composition comprising a nucleic acid molecule comprising a nucleotide sequence encoding at least one AAV donor polynucleotide can be a composition comprising Adeno-associated virus (AAV) viral vector particles comprising at least one nucleic acid molecule comprising a polynucleotide, wherein the polynucleotide comprises a nucleotide sequence encoding at least one therapeutic protein. Accordingly, the present disclosure provides methods of treating at least one disease in a subject, the methods comprising administering to the subject: a) at least one therapeutically effective amount of AAV viral vector particles comprising at least one nucleic acid molecule comprising a AAV donor polynucleotide, wherein the polynucleotide comprises a nucleotide sequence encoding at least one therapeutic protein; and b) at least one therapeutically effective amount of a composition comprising a targeting gRNA pair and a nucleic acid molecule comprising a nucleotide sequence encoding at least one Cas-CLOVER. In certain methods, the nucleic acid molecule comprising a nucleotide sequence encoding at least one Cas-CLOVER is an mRNA.

In a non-limiting example, AAV viral vector particles comprising at least one nucleic acid molecule comprising a transposon, wherein the transposon comprises a nucleotide sequence encoding at least one therapeutic protein, wherein the therapeutic protein is hPAH, can comprise, consist essentially of, or consist of a nucleic acid sequence at least 65%, 70%, 75%, 80%, 85%, 90%, 95%, 96%, 97%, 98%, or 99% (or any percentage in between) identical to any one of SEQ ID NOs: 18 and 21. In some embodiments, AAV viral vector particles comprising at least one nucleic acid molecule comprising a transposon, wherein the transposon comprises a nucleotide sequence encoding at least one therapeutic protein, wherein the therapeutic protein is hPAH, comprises, consists essentially of, or consists of the nucleic acid sequence set forth in SEQ ID NOs: 18 or 21.

In some aspects of the preceding methods, a composition comprising a nucleic acid molecule comprising a AAV donor polynucleotide, wherein the polynucleotide comprises a nucleotide sequence encoding at least one therapeutic protein and a composition comprising a nucleic acid molecule comprising a targeting gRNA pair and a nucleotide sequence encoding at least one Cas-CLOVER can be administered concurrently. In some aspects, a composition comprising a nucleic acid molecule comprising a AAV donor polynucleotide, wherein the polynucleotide comprises a nucleotide sequence encoding at least one therapeutic protein and a composition comprising a nucleic acid molecule comprising a targeting gRNA pair and a nucleotide sequence encoding at least one Cas-CLOVER se can be administered sequentially. In some aspects, a composition comprising a nucleic acid molecule comprising a AAV donor polynucleotide, wherein the polynucleotide comprises a nucleotide sequence encoding at least one therapeutic protein and a composition comprising a nucleic acid molecule comprising a targeting gRNA pair and a nucleotide sequence encoding at least one Cas-CLOVER can be administered in temporal proximity.

As used herein, the term “temporal proximity” refers to that administration of one therapeutic composition (e.g., a composition comprising a transposon) occurs within a time period before or after the administration of another therapeutic composition (e.g., a composition comprising a transposase), such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

In a non-limiting example, the present disclosure provides methods of treating PKU in a subject, the methods comprising administering to the subject: a) at least one therapeutically effective amount AAV viral vector particles comprising at least one nucleic acid molecule comprising a AAV donor polynucleotide, wherein the polynucleotide comprises a nucleotide sequence encoding at least one therapeutic protein; and b) at least one therapeutically effective amount of LNPs of the present disclosure, wherein the LNPs comprise at least one targeting gRNA pair and at least one RNA molecule comprising a nucleotide sequence encoding at least one Cas-CLOVER. In some aspects, the at least one therapeutic protein can comprise human phenylalanine hydroxylase (hPAH) polypeptide.

In some aspects of the treatment methods of the present disclosure, the administration of the at least one composition and/or nanoparticle of the present disclosure to a subject can result in the expression of an exogenous protein (e.g. a therapeutic protein, a transposase, etc.) in at least one organ and/or tissue in the subject.

In some aspects, the administration of the at least one composition and/or nanoparticle of the present disclosure results in the expression of the exogenous protein in at least about 10%, or at least about 15%, or at least bout 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99% of the cells in the tissue and/or organ.

In some aspects, the administration of the at least one composition and/or nanoparticle of the present disclosure results in the expression of the exogenous protein in at least about 10%, or at least about 15%, or at least bout 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99% of a specific subset or subsets of cells in the tissue and/or organ.

In some aspects, the administration of the at least one composition and/or nanoparticle of the present disclosure results in the expression of the exogenous protein for at least about 1 day, or at least about 2 days, or at least about 3 days, or at least about 4 days, or at least about 5 days, or at least about 6 days, or at least about 7 days, or at least about 8 days, or at least about 9 days, or at least about 10 days in the tissue and/or organ.

In some aspects, the administration of the at least one composition and/or nanoparticle of the present disclosure results in the expression of the exogenous protein for at least about 1 day, or at least about 2 days, or at least about 3 days, or at least about 4 days, or at least about 5 days, or at least about 6 days, or at least about 7 days, or at least about 8 days, or at least about 9 days, or at least about 10 days in a specific subset or subsets of cells in the tissue and/or organ.

In some aspects, the administration of the at least one composition and/or nanoparticle of the present disclosure results in the expression of the exogenous protein for no more than about 1 day, or no more than about 2 days, or no more than about 3 days, or no more than about 4 days, or no more than about 5 days, or no more than about 6 days, or no more than about 7 days, or no more than about 8 days, or no more than about 9 days, or no more than about 10 days in the tissue and/or organ.

In some aspects, the administration of the at least one composition and/or nanoparticle of the present disclosure results in the expression of the exogenous protein for no more than about 1 day, or no more than about 2 days, or no more than about 3 days, or no more than about 4 days, or no more than about 5 days, or no more than about 6 days, or no more than about 7 days, or no more than about 8 days, or no more than about 9 days, or no more than about 10 days in a specific subset or subsets of cells in the tissue and/or organ.

The present disclosure also provides methods of treating at least one disease in a subject, wherein the subject has a missense mutation (for example F263S) that inactivates the PAH gene and these subjects exhibit classic PKU, with elevated blood phenylalanine (Phe) levels, cognitive deficiencies, and maternal PKU syndrome. See, for example, Charron C. et al., Molecular Therapy Vol. 11, Supplement 1, May 2005, S163-S164.

In some aspects, the present disclosure provides methods of treating PKU in a subject in need thereof, the methods comprising administering to the subject: a) at least one therapeutically effective amount AAV viral vector particles comprising at least one nucleic acid molecule comprising a AAV donor polynucleotide, wherein the polynucleotide comprises a nucleotide sequence encoding at least one therapeutic protein; and b) at least one therapeutically effective amount of LNPs of the present disclosure, wherein the LNPs comprise at least one targeting gRNA pair and at least one RNA molecule comprising a nucleotide sequence encoding at least one Cas-CLOVER. In some aspects, the at least one therapeutic protein can comprise human phenylalanine hydroxylase (hPAH) polypeptide.

### Cells and Modified Cells of the Disclosure

In another aspect, provided herein are cell and modified cells comprising the vectors and compositions described herein. Cells and modified cells of the disclosure can be mammalian cells. Preferably, the cells and modified cells are human cells. In one aspect, the cells targeted for modification using the LNP compositions of the present disclosure are hepatocytes, a hepatic stellate cells, Kupffer cells or liver sinusoidal endothelial cells. In one embodiment, the LNP compositions comprise at least one targeting gRNA pair and at least one RNA molecule comprising a nucleotide sequence encoding at least one Cas-CLOVER and the modified cells are generated in vivo. In one embodiment, the polynucleotide comprises a nucleotide sequence encoding a therapeutic gene operatively linked to a liver-specific promoter.

Cells and modified cells of the disclosure can be somatic cells. Cells and modified cells of the disclosure can be differentiated cells. Cells and modified cells of the disclosure can be autologous cells or allogenic cells. Allogeneic cells are engineered to prevent adverse reactions to engraftment following administration to a subject. Allogeneic cells may be any type of cell. Allogenic cells can be stem cells or can be derived from stem cells. Allogeneic cells can be differentiated somatic cells.

### Nucleic Acid Molecules

Nucleic acid molecules of the disclosure encoding a therapeutic protein can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the disclosure can include nucleic acid molecules comprising an open reading frame (ORF), optionally, with one or more introns, e.g., but not limited to, at least one specified enzymatically active portion of a therapeutic protein; nucleic acid molecules comprising the coding sequence for a therapeutic protein and nucleic acid molecules which comprise a nucleotide sequence substantially different from those described above but which, due to the degeneracy of the genetic code, still encode the therapeutic protein as described herein and/or as known in the art. Of course, the genetic code is well known in the art. Thus, it would be routine for one skilled in the art to generate such degenerate nucleic acid variants that code for a specific protein scaffold of the present disclosure. See, e.g., Ausubel, et al., supra, and such nucleic acid variants are included in the present disclosure.

As indicated herein, nucleic acid molecules of the disclosure which comprise a nucleic acid molecule encoding a therapeutic protein can include, but are not limited to, those encoding the amino acid sequence of an enzymatically active fragment of a therapeutic protein, by itself; the coding sequence for the entire a therapeutic protein or a portion thereof; the coding sequence for a therapeutic protein, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding a therapeutic protein can be fused to a marker sequence, such as a sequence encoding a peptide that facilitates purification of the fused therapeutic protein.

### Construction of Nucleic Acids

The isolated nucleic acids of the disclosure can be made using (a) recombinant methods, (b) synthetic techniques, (c) purification techniques, and/or (d) combinations thereof, as well-known in the art.

The nucleic acids can conveniently comprise sequences in addition to a polynucleotide of the present disclosure. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra, or Sambrook, supra).

### Recombinant Methods for Constructing Nucleic Acids

The isolated nucleic acid compositions of this disclosure, such as RNA, cDNA, genomic DNA, or any combination thereof, can be obtained from biological sources using any suitable cloning methodology known to those of skill in the art. In some aspects, oligonucleotide probes that selectively hybridize, under stringent conditions, to the polynucleotides of the present disclosure are used to identify the desired sequence in a cDNA or genomic DNA library. The isolation of RNA, and construction of cDNA and genomic libraries are well known to those of ordinary skill in the art. (See, e.g., Ausubel, supra, or Sambrook, supra).

### Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra, or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The disclosure further provides recombinant expression cassettes comprising a nucleic acid of the disclosure. A nucleic acid sequence of the disclosure, for example, a cDNA or a genomic sequence encoding a protein scaffold of the disclosure, can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some aspects, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

### Expression Vectors and Host Cells

The disclosure also provides vectors that include isolated nucleic acid molecules of the disclosure, host cells that are genetically engineered with the recombinant vectors, and the production of at least one therapeutic protein by recombinant techniques, as is well known in the art. See, e.g., Sambrook, et al., supra, Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g. UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Streptoalloteichus hindustanus bleomycin gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotes (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD 19, CD271, CD34, CD22, CD20, CD33, CD52, or any combination thereof. Cell surface markers further include the suicide gene marker RQR8 (Philip B et al. Blood. 2014 Aug. 21; 124 (8): 1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCE, RAD51C, GCS, MDR1, ALDH1, NKX2.2, or any combination thereof.

At least one protein scaffold of the disclosure can be expressed in a modified form, such as a fusion protein, and can include not only secretion signals, but also additional heterologous functional regions. For instance, a region of additional amino acids, particularly charged amino acids, can be added to the N-terminus of a protein scaffold to improve stability and persistence in the host cell, during purification, or during subsequent handling and storage. Also, peptide moieties can be added to a protein scaffold of the disclosure to facilitate purification. Such regions can be removed prior to final preparation of a protein scaffold or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid molecule encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g, ATCC CRL 1650), COS-7 (e.g, ATCC CRL-1651), HEK293, BHK21 (e.g, ATCC CRL-10), CHO (e.g, ATCC CRL 1610) and BSC-1 (e.g, ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a preferred aspect, the recombinant cell is a P3X63Ab8.653 or an SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphogly cerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly(A) addition site), and transcriptional terminator sequences. See, e.g., Ausubel et al., supra, Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenylation or transcription terminator sequences are typically incorporated into the vector. An example of a terminator sequence is the polyadenylation poly(A) sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

### Amino Acid Codes

The amino acids that make up proteins of the disclosure are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994). A therapeutic protein of the disclosure can include one or more amino acid substitutions, deletions or additions, from spontaneous or mutations and/or human manipulation, as specified herein. Amino acids in a therapeutic protein of the disclosure that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one neutralizing activity. Sites that are critical for maintaining the activity of the therapeutic protein can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

As those of skill will appreciate, the disclosure includes at least one biologically active therapeutic protein of the disclosure. Biologically active therapeutic protein have a specific activity at least 20%, 30%, or 40%, and, preferably, at least 50%, 60%, or 70%, and, most preferably, at least 80%, 90%, or 95%-99% or more of the specific activity of the native (non-synthetic), endogenous or related and known protein scaffold. Methods of assaying and quantifying measures of enzymatic activity and substrate specificity are well known to those of skill in the art.

Fatty acids and fatty acid esters suitable for modifying therapeutic proteins of the disclosure can be saturated or can contain one or more units of unsaturation. Fatty acids that are suitable for modifying protein scaffolds of the disclosure include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-A9-octadecanoate (C18, oleate), all cis-Δ5.8.1 1.14-eicosatetraenoate (C20, arachidonate), octanedioic acid, tetradecanedioic acid, octadecanedioic acid, docosanedioic acid, and the like. Suitable fatty acid esters include monoesters of dicarboxylic acids that comprise a linear or branched lower alkyl group. The lower alkyl group can comprise from one to about twelve, preferably, one to about six, carbon atoms.

The modified therapeutic proteins and fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH2)3-, —NH—(CH2)6-NH—, —(CH2)2-NH— and —CH2-O—CH2-CH2-O—CH2-CH2O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified therapeutic proteins of the disclosure can be produced by reacting a protein scaffold or fragment with a modifying agent. For example, the organic moieties can be bonded to the protein scaffold in a non-site specific manner by employing an amine-reactive modifying agent, for example, an NHS ester of PEG. Modified therapeutic proteins and fragments comprising an organic moiety that is bonded to specific sites of a protein scaffold of the disclosure can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6 (10): 2233-2241 (1997); Itoh et al., Bioorg. Chem., 24 (1): 59-68 (1996); Capellas et al., Biotechnok Bioeng., 56 (4): 456-463 (1997)), and the methods described in Hermanson, G. T., Bioconjugate Techniques, Academic Press: San Diego, Calif. (1996).

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system.

For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific.

The term “homologous to” as used herein in the context of a polynucleotide (e.g., a gRNA) being homologous to a target sequence refers to sufficient homology for the polynucleotide to bind the target sequence.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode the same protein.

Probes of the disclosure may comprise a single stranded nucleic acid that can hybridize to a target sequence under stringent hybridization conditions. Thus, nucleic acids of the disclosure may refer to a probe that hybridizes under stringent hybridization conditions.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “variant” when used to describe a nucleic acid, refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table 2.

TABLE 2
Conservative Substitutions I

| | |
|---|---|
| Side chain characteristics | Amino Acid |

| | | |
|---|---|---|
| Aliphatic | Non-polar | G A P I L V F |
| | Polar - uncharged | C S T M N Q |
| | Polar - charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table 3

TABLE 3
Conservative Substitutions II

| | |
|---|---|
| Side Chain Characteristic | Amino Acid |

| | | |
|---|---|---|
| Non-polar (hydrophobic) | Aliphatic: | A L I V P |
| | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table 4.

TABLE 4
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring.

Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### EXAMPLES

### Example 1: Construction of Exemplary AAV Donor Polynucleotides for Site Specific Genomic Integration

### A. Promoterless Bi-directional Luciferase Expression AAV Donor Polynucleotide

The following is a non-limiting example demonstrating the construction of an exemplary AAV donor polynucleotide comprising a promoterless bidirectional luciferase expression cassette for measuring site-specific integration into intron 3 of the albumin gene, and luciferase expression levels controlled by endogenous albumin promoter.

A general schematic diagram of AAV donor polynucleotide comprising a bidirectional luciferase reporter cassette is shown in FIG. 1A. As shown in FIG. 1A, the AAV donor polynucleotide comprises in the 5′ to 3′ direction: a) a first AAV ITR sequence (SEQ ID NO: 12); b) a first target sequence homologous to an upstream portion of intron 3 of the albumin gene (SEQ ID NO: 13); c) a splice acceptor sequence (SEQ ID NO: 14); d) a P2A sequence (SEQ ID NO: 15); e) a sequence encoding luciferase (SEQ ID NO: 16); f) a polyadenylation (poly(A)) sequence (SEQ ID NO: 17); g) a DNA spacer sequence (SEQ ID NO: 18); h) an inverted poly(A) sequence (SEQ ID NO: 19); i) an inverted sequence encoding luciferase (SEQ ID NO: 20); j) an inverted P2A sequence (SEQ ID NO: 21) k) an inverted splice acceptor sequence (SEQ ID NO: 22); 1) a second target sequence homologous to a downstream portion of Intron 3 of the albumin gene (SEQ ID NO: 23); and m) a second AAV ITR sequence (SEQ ID NO: 24).

The AAV donor DNA was constructed using Gibson assembly from a single DNA fragment comprising the bidirectional SA-P2A-luciferase gene-poly(A); and ii) a vector backbone containing Cas-CLOVER linearization sites (first and second target sequences) flanked by AAV ITRs. The complete nucleotide sequence of the AAV donor DNA comprising a promoterless bidirectional luciferase expression cassette is provided in SEQ ID NO: 6.

### B. Promoterless Bi-directional Phenylalanine Hydroxylase (PAH) Expression AAV Donor Vector Polynucleotide

The following is a non-limiting example demonstrating the construction of an exemplary AAV donor polynucleotide comprising a promoterless bidirectional PAH expression cassette for achieving site-specific integration into intron 3 of the albumin gene, and PAH expression levels controlled by endogenous albumin promoter.

A general schematic diagram of AAV donor polynucleotide comprising a bidirectional PAH expression cassette is shown in FIG. 1B. As shown in FIG. 1B, the AAV donor polynucleotide comprises in the 5′ to 3′ direction: a) a first AAV ITR sequence (SEQ ID NO: 12); b) a first target sequence homologous to an upstream portion of intron 3 of the albumin gene (SEQ ID NO: 13); c) a splice acceptor sequence (SEQ ID NO: 14); d) a P2A sequence (SEQ ID NO: 15); e) a sequence encoding a codon optimized and modified PAH gene (SEQ ID NO: 25); f) a polyadenylation (poly(A)) sequence (SEQ ID NO: 17); g) a DNA spacer sequence (SEQ ID NO: 18); h) an inverted poly(A) sequence (SEQ ID NO: 19); i) an inverted sequence encoding a codon optimized and modified PAH gene (SEQ ID NO: 26); j) an inverted P2A sequence (SEQ ID NO: 21) k) an inverted splice acceptor sequence (SEQ ID NO: 22); 1) a second target sequence homologous to a downstream portion of intron 3 of the albumin gene (SEQ ID NO: 23); and m) a second AAV ITR sequence (SEQ ID NO: 24).

The nucleotide sequence of the mRNA encoding human PAH sequence (GenBank U49897.1) was codon optimized to improve PAH expression using computer algorithms in accordance with the various manufacturer's instructions (e.g., ThermoFisher, Blue Heron Biotech, and Integrated DNA Technologies). After codon optimization, the optimized sequence was further modified to: i) remove any undesired restriction enzyme recognition sites; and ii) putative cryptic splice sites for cloning the modified sequences into AAV donor vectors. The sequence also comprises an N-terminal hemagglutinin tag.

The AAV donor polynucleotide DNA was constructed using Gibson assembly from a single DNA fragment comprising the bidirectional SA-P2A-HA-PAH gene-poly(A); and ii) a vector backbone containing Cas-CLOVER linearization sites (first and second target sequences) flanked by AAV ITRs. The complete nucleotide sequence of the AAV donor polynucleotide comprising a promoterless bidirectional PAH expression cassette is provided in SEQ ID NO: 8.

### C. TTRe-PAH Reporter AAV Donor Polynucleotide

The following is a non-limiting example demonstrating the construction of an exemplary AAV donor polynucleotide comprising a TTRe-PAH promoter expression cassette for achieving site-specific DNA integration into intron 3 of the albumin gene, and PAH expression levels using a heterologous promoter.

A general schematic diagram of AAV donor polynucleotide comprising a bidirectional PAH expression cassette is shown in FIG. 1C. As shown in FIG. 1C, the AAV donor polynucleotide comprises in the 5′ to 3′ direction: a) a first AAV ITR sequence (SEQ ID NO: 12); b) a first target sequence homologous to an upstream portion of intron 3 of the albumin gene (SEQ ID NO: 13); c) a TTRe promoter sequence; d) a splice acceptor sequence (SEQ ID NO: 14); e) a P2A sequence (SEQ ID NO: 15); f) a sequence encoding a codon optimized and modified PAH gene (SEQ ID NO: 25); g) a 3′UTR sequence; h) a polyadenylation (poly(A)) sequence (SEQ ID NO: 17); i) a DNA spacer sequence (SEQ ID NO: 18); j) a second target sequence homologous to a downstream portion of intron 3 of the albumin gene (SEQ ID NO: 23); and k) a second AAV ITR sequence (SEQ ID NO: 24).

The AAV donor PAH reporter polynucleotide was constructed using Gibson assembly from a single DNA fragment comprising the TTRe promoter-HA-PAH gene-poly(A); and ii) a vector backbone containing Cas-CLOVER linearization sites (first and second target sequences) and the UTR flanked by AAV ITRs. The complete nucleotide sequence of the first AAV PAH reporter donor polynucleotide is provided in SEQ ID NO: 10.

### Example 2—Preparation of 5′-Capped mRNA Encoding Cas-CLOVER for Encapsulation in LNP Compositions

The following is a non-limiting example demonstrating the preparation of an exemplary mRNA encoding Cas-CLOVER that may be incorporated in LNP compositions for use in combination in methods with AAV donor DNA polynucleotides and vectors of the present disclosure.

The DNA plasmid pRTb_Cas-CLOVERv3 encodes Cas-CLOVER (SEQ ID NO: 1) comprising an N-terminal SV40 nuclear localization signal (NLS) and containing the 5′ UTR of the human beta-globin gene (HBB) and the 3′ UTR of the human cytochrome b-245 alpha chain gene (CYBA).

This plasmid was used as a template for in vitro transcription reactions to produce mRNA encoding Cas-CLOVERv3 further comprising a 5′-CAP.

Briefly, approximately 300 μg of supercoiled pRTb_Cas-CLOVERv3 was added to a 15 mL conical comprising 300 μL CutSmart Buffer, 60 μL of the restriction enzyme Bbsl-HF (New England Biolabs, Cat #R3539M) in 3000 μL total volume. The plasmid DNA was linearized by incubating at 37° C. overnight to ensure complete digestion.

The linearized plasmid was purified using a DNA QIAquick PCR purification kit (Qiagen, Cat #28106) according to the manufacturer's instructions, and the purified DNA eluted in 900 μL of nuclease-free water (ThermoFisher, Cat #AM9937). The DNA concentration and purity of the eluate was determined using a NanoDrop microvolume spectrophotometer (ThermoFisher) in accordance with the manufacturer's instructions.

The purified plasmid was used as a DNA template to produce mRNA using the custom in vitro transcription mMESSAGE mMACHINE T7 Transcription Kit (ThermoFisher, Cat #AM1345B001) in accordance with internal, Quality-controlled manufacturing batch records. Briefly, 100 mM stocks of the nucleotides GTP (ThermoFisher, Cat #R0481), ATP (ThermoFisher, Cat #R0481), CTP (ThermoFisher, Cat #R0481), and N1MeΨTP (N1-Methylpseudouridine-5′-Triphosphate) (TriLink, Cat #N-1081) and CleanCap Reagent AG (m7G(5′)ppp(5′)(2′OMeA)pG; Trilink, Cat #N-7113) were prepared. 1,485 μL each of ATP, UTP and 5MeC and 1,188 μL each of GTP and CleanCap Reagent AG were blended.

153 μg of linearized pRTb_Cas-CLOVERv3 DNA, 1,800 μL of 10×T7 RXN Buffer (ThermoFisher, Cat #AM1345B001), 1,800 μL of T7 Enzyme mix (ThermoFisher, Cat #AM1345B001), and 6,831 μL of the NTP and cap blend were added to a 50 mL conical (18,000 μL final volume), and incubated at 37° C. for 3 hours. A 900 μL aliquot of DNaseI enzyme (ThermoFisher, Cat #AM1345B001) was added and the tube further incubated at 37° C. for 15 min to degrade the DNA template.

A poly(A) tail was post-enzymatically added to the 3′ end of the 5′-CleanCap®-Cas-CLOVER-N1MeΨmRNA. 18,000 μL of 5×EPAP Buffer (ThermoFisher, Cat #AM1345B001), 9,000 μL of 25 mM MnCl₂ (ThermoFisher, Cat #AM1345B001), 9,000 μL ATP Solution (ThermoFisher, Cat #AM1345B001), and 3,000 μL E-PAP (ThermoFisher, Cat #AM1345B001), were added to the IVT reaction (90,000 μL total volume), and incubated at 37° C. for 1 hour. The bulk E-PAP reaction was subsequently divided into three 125 mL PETG bottles in 30 mL aliquots.

The 5′-CleanCap®-Cas-CLOVER-poly(A)-N1MeΨ mRNA was purified using a RNeasy Maxi Purification Kit (Qiagen, Cat #75162) according to the manufacturer's instructions. Briefly, a working stock of Buffer RLT was formulated using 178.2 mL of Buffer RLT (Qiagen, Cat #75162) with 1,800 μL of 2-mercaptoethanol (Sigma, Cat #M3148). 52.2 mL of the BME+RLT solution and 37.8 mL of 100% EtOH (ThermoFisher, Cat #BP2818) were added to each 30 mL mRNA aliquot. The purified mRNA product was eluted in 52.5 mL of nuclease-free water, and the bulk product stored at −80° C. The DNA linearization, IVT, and mRNA purification process is repeated until the target yield is reached.

Bulk mRNA lots were analyzed using gel electrophoresis before combination in a 500 mL PETG bottle, and sampled for concentration readings using the NanoDrop. Lithium Chloride 5× (ThermoFisher, Cat #AM1345B001) was added to the pooled mRNA in the amount of ⅓ of the total volume of the mRNA, then divided into equal 40 mL aliquots in 50 mL conical tubes and incubated at −20° C. for 45 minutes. Directly following incubation, the conical tubes are centrifuged at 14,000 g for 30 minutes at 4° C. The mRNA pellet is washed using 70% EtOH (ThermoFisher, Cat #BP8201) three times.

The washed mRNA pellets are dried, then resuspended in nuclease-free water. The mRNA concentration was determined using the NanoDrop, and additional nuclease-free water was added as necessary to further dilute the product to the target concentration. The mRNA is sterile filtered using a 0.22 μm PES SteriCup Filter (Sigma, Cat #52GPU05RE) before the final mRNA concentration and purity are measured on the NanoDrop.

### Example 3—Preparation of an LNP Composition Comprising a 5′-Capped mRNA Encoding Cas-CLOVER and a gRNA Pair

The following is a non-limiting example that provides exemplary methods for formulating an LNP composition comprising a 5′-capped mRNA encoding Cas-CLOVER and a gRNA pair for use in combination with the AAV donor DNA polynucleotides disclosed herein.

Individual 25 mg/ml stock solutions were prepared by solubilizing the lipids in 200-proof HPLC-grade ethanol and stock solutions were stored at −80° C. until formulated. At the time of formulation, the lipid stock solutions were briefly allowed to equilibrate to room temp and then placed on a hot plate maintained at a temperature range of 50-55° C. Subsequently, the hot lipid stock solutions were combined to yield desired final mol percentages.

A 1 mg/ml solution of the 5′CleanCap-N1-CC mRNA prepared in Example 2 to be incorporated into the LNPs was added to 150 mM sodium acetate buffer (pH 5.2) to form a stock solution and kept on ice. A 1 mg/ml solution of the gRNA Pair dissolved in RNAse free water to be incorporated into the LNPs was added to 150 mM sodium acetate buffer (pH 5.2) to form a gRNA stock solution and kept on ice. mRNA and gRNA stock solutions were mixed at a 3:1 ratio to form a nucleic acid stock solution. The lipid phase was mixed with the aqueous mRNA/gRNA phase inside a microfluidic chip using aNanoAssemblr® instrument (Precision Nanosystems, Vancouver, BC, Canada) according to the manufacturer's instructions to form LNP compositions comprising encapsulated Cas-CLOVER mRNAs and a targeting gRNA pair. Nanoassemblr process parameters for mRNA encapsulation were at a flow rate of 20 ml/min and at a lipid:RNA ratio (v/v) of 1:3.

The resultant Cas-CLOVER mRNA-gRNA pair LNP compositions were then transferred to a Repligen Float-A-Lyzer dialysis device-having a molecular weight cut off (MWCO) of 8-10 kDa (Spectrum Chemical Mfg. Corp, CA, USA) and processed by dialysis against 25 mM sodium acetate (dialysate:dialysis buffer volume at least 1:200 v/v), pH 5.5 overnight at 4° C. (or alternatively room temperature for at least 4 hours), to remove the 25% ethanol and achieve a complete buffer exchange. In applicable, the LNP compositions were further concentrated using an Amicon® Ultra-4 centrifugal filter unit, MWCO-30 kDa (Millipore Sigma, USA) spun at ~4100×g in an ultracentrifuge. Sucrose was added to a final concentration of 5% (w/v) to the mRNA LNPs which were then stored at 4° C. or frozen at −80° C. until further use.

The average particle size diameter of the LNPs ranged from approximately 85-10⁵ nm.

### Example 4—In Vivo Site-specific Integration of AAV Donor Polynucleotides Using Cas-CLOVER in Hepatocytes

The following is a non-limiting example demonstrating the AAV donor DNA polynucleotides can be site-specifically integrated in vivo into Intron 3 of the albumin gene.

Adult (10-12 weeks old) C57BL/6 mice (n=3 mice per group) were administered the following treatments:
Treatment #1: Vehicle;
Treatment #2: Promoterless bidirectional luciferase expression AAV viral vector particles (Example 1A);
Treatment #3: Promoterless bidirectional luciferase expression AAV viral vector particles (1E12 vg/kg) in combination with 1.5 mg/kg LNPs encapsulating mRNA encoding Cas-CLOVER and a gRNA pair targeting intron 3 of the murine albumin gene;
Treatment #4: Promoterless bidirectional luciferase expression AAV viral vector particles (5E12 vg/kg) in combination with 1.5 mg/kg LNPs encapsulating mRNA encoding Cas-CLOVER and a gRNA pair targeting intron 3 of the murine albumin gene;
Treatment #5: Promoterless bidirectional luciferase expression AAV viral vector particles (1E13 vg/kg) in combination with 1.5 mg/kg LNPs encapsulating mRNA encoding Cas-CLOVER and a gRNA pair targeting intron 3 of the murine albumin gene; and
Treatment #6: Promoterless bidirectional luciferase expression AAV viral vector particles (3E13 vg/kg) in combination with 1.5 mg/kg LNPs encapsulating mRNA encoding Cas-CLOVER and a gRNA pair targeting intron 3 of the murine albumin gene.

The promoterless bidirectional luciferase expression AAV viral vector particles were AAV viral vector particles comprising the nucleic acid of SEQ ID NO: 6 and were administered retro-orbitally. The bidirectional cassette allows for albumin regulated luciferase mRNA expression regardless of the AAV donor DNA polynucleotide to be integrated in the forward or reverse configuration.

The LNP composition comprising a gRNA pair and the mRNA encoding Cas-CLOVER prepared in Example 2 comprised the following components: ssPalmO-Ph-P4C2, DOPC, DSPC, Cholesterol and DMG-PEG2000 at a molar ratio of 54:5:5:35:1 and had a lipid: RNA ratio 100:1 (w/w). LNP compositions were administered by tail vein injection.

After fourteen days post-administration, whole body luminescence imaging (BLI) was determined for control and treated animals, and liver biopsies and blood samples were collected from treated mice to determine percent albumin gene editing, percent AAV donor DNA integration, and luciferase expression levels relative to albumin mRNA expression levels. To isolate genomic DNA, liver samples were mixed with lysis buffer (15 mg of tissue in 200 μL of lysis buffer+10 μL Proteinase K) and pulverized in a TissueLyser II (Qiagen) using Triple-Pure zirconium beads (Fisher Scientific). Homogenized tissue was then incubated at 56° C. for 30 minutes, and column-purified using a Monarch Genomic DNA Purification kit (New England Biolabs) in accordance with manufacturer's instructions. Final DNA elution was performed in 50 μL of elution buffer (10 mM Tris-Cl, pH 8.5). The concentration and purity of DNA samples were assessed by measuring absorbance at 260 and 280 nm using a Nanodrop device. DNA samples were used for albumin editing and transgene integration quantification. The extent of albumin editing observed for Cas-CLOVER mRNA delivered to the mice was measured by Droplet Digital PCR (ddPCR) using Drop-off assays that contain a fluorescent probe that hybridizes with Cas-CLOVER target site. The extent of transgene integration at the albumin site was measured by ddPCR using a probe-based detection scheme. One primer binds within the luciferase transgene was paired with a primer that binds the albumin genomic DNA near the Cas-CLOVER cut site.

To isolate mRNA, liver samples were mixed with lysis buffer (25 mg of tissue in 300 μL of lysis buffer+15 μL Proteinase K) and pulverized in a TissueLyser II (Qiagen) using Triple-Pure zirconium beads (Fisher Scientific). Homogenized tissue was then incubated at RT for 30 minutes, and column-purified using a Quick RNA Miniprep Plus kit (Zymo Research) in accordance with manufacturer's instructions. Final RNA elution was performed in 50 μL of DNase/RNase-free water. The concentration and purity of DNA samples were assessed by measuring absorbance at 260 and 280 nm using a Nanodrop device. RNA samples were used for luciferase mRNA quantification by quantitative real time polymerase chain reaction (RT-qPCR).

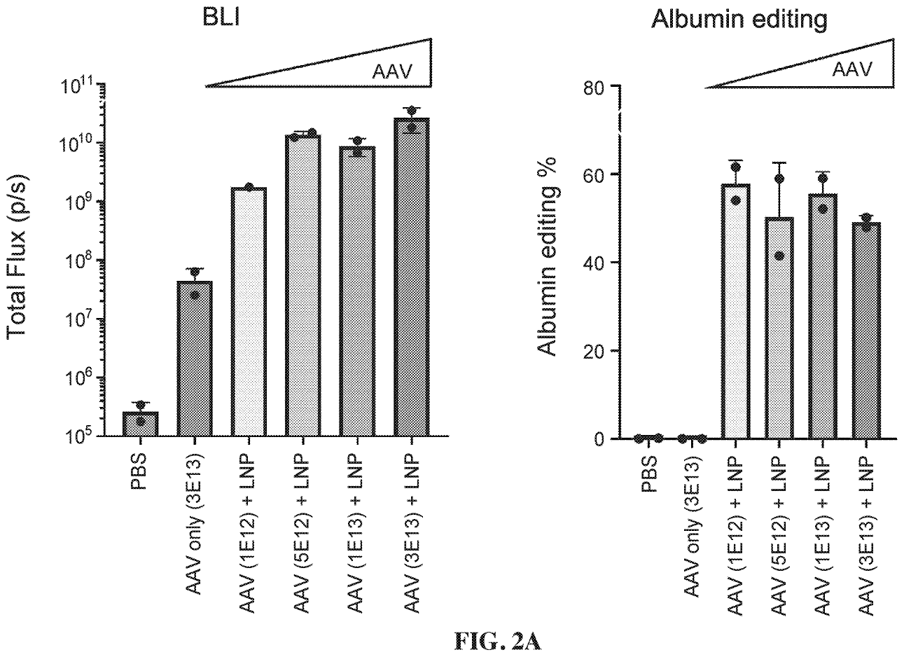

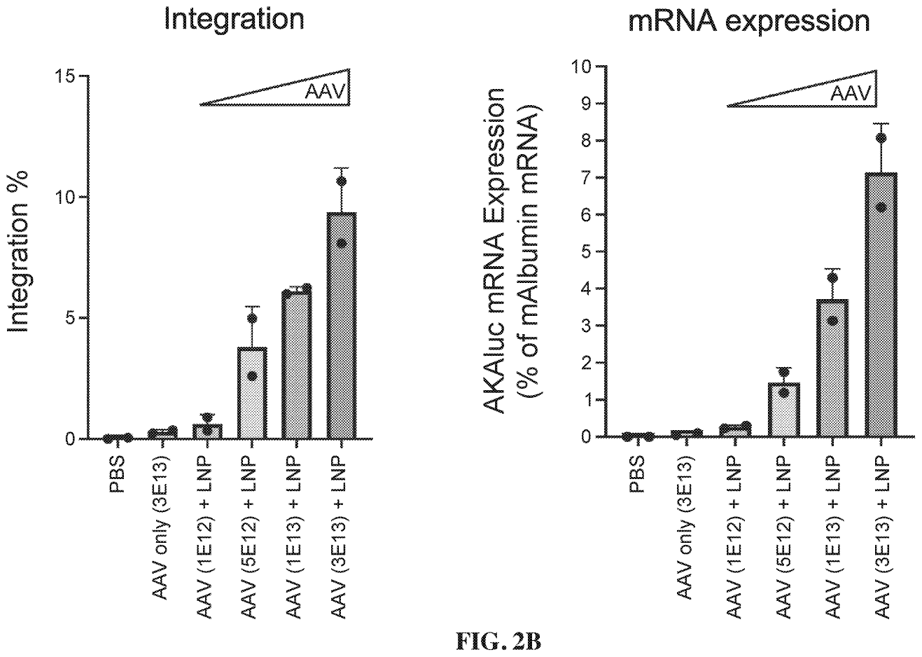

The results are described in FIGS. 2A and 2B. The expression of luciferase was shown by BLI (FIG. 2A) and the percent integration and luciferase mRNA expression (FIG. 2B) found to be dose-dependent; and the addition of LNPs comprising an albumin targeting gRNA pair and Cas-CLOVER mRNA significantly increased luciferase mRNA expression, percent integration (FIG. 2B), and the percent of albumin edited haploid genomes (FIG. 2A) compared to the levels observed in mice treated with vehicle or AAV donor DNA alone. These observations indicate that the integration is site-specific, with expression driven by the endogenous albumin gene.

### Example 5—In Vivo Site-specific Integration of AAV Donor Polynucleotides Using Cas-CLOVER and Expression of PAH in Hepatocytes

The following is a non-limiting example demonstrating the AAV donor DNA polynucleotides can be site-specifically integrated in vivo into Intron 3 of the albumin gene of hepatocytes in adult mice and expression of PAH in integrated hepatocytes.

Adult (10-12 weeks old) C57BL/6 mice (n=3 mice per group) were administered the following treatments:
Treatment #1: Vehicle;
Treatment #2: Promoterless bidirectional PAH reporter AAV viral vector particles (Example 1B);
Treatment #3: Promoterless bidirectional PAH expression AAV viral vector particles (1E12 vg/kg) in combination with 1.5 mg/kg LNPs encapsulating mRNA encoding Cas-CLOVER and a gRNA pair targeting intron 3 of the murine albumin gene;
Treatment #4: Promoterless bidirectional PAH expression AAV viral vector particles (3E12 vg/kg) in combination with 1.5 mg/kg LNPs encapsulating mRNA encoding Cas-CLOVER and a gRNA pair targeting intron 3 of the murine albumin gene;
Treatment #5: Promoterless bidirectional PAH expression AAV viral vector particles (1E13 vg/kg) in combination with 1.5 mg/kg LNPs encapsulating mRNA encoding Cas-CLOVER and a gRNA pair targeting intron 3 of the murine albumin gene; and
Treatment #6: TTRe-PAH expression AAV viral vector particles (Example 1C);
Treatment #7: TTRe-PAH expression AAV viral vector particles (1E12 vg/kg) in combination with 1.5 mg/kg LNPs encapsulating mRNA encoding Cas-CLOVER and a gRNA pair targeting intron 3 of the murine albumin gene;
Treatment #8: TTRe-PAH expression AAV viral vector particles (3E12 vg/kg) in combination with 1.5 mg/kg LNPs encapsulating mRNA encoding Cas-CLOVER and a gRNA pair targeting intron 3 of the murine albumin gene; and
Treatment #9: TTRe-PAH expression AAV viral vector particles (1E13 vg/kg) in combination with 1.5 mg/kg LNPs encapsulating mRNA encoding Cas-CLOVER and a gRNA pair targeting intron 3 of the murine albumin gene.

The promoterless bidirectional PAH reporter AAV viral vector particles were AAV viral vector particles comprising the nucleic acid of SEQ ID NO: 8 and were administered retro-orbitally. The bidirectional cassette allows for albumin regulated PAH mRNA expression regardless of the AAV donor polynucleotide being integrated in the forward or reverse configuration.

TTRe-PAH expression AAV viral vector particles were AAV viral vector particles comprising the nucleic acid of SEQ ID NO: 10 and were administered retro-orbitally. Expression of PAH is controlled by the heterologous TTRe promoter.

The LNP composition comprising a gRNA pair and the mRNA encoding Cas-CLOVER prepared in Example 2 comprised the following components: ssPalmO-Ph-P4C2, DOPC, DSPC, Cholesterol and DMG-PEG2000 at a molar ratio of 54:5:5:35:1 and had a lipid:RNA ratio 100:1 (w/w). LNP compositions were administered by tail vein injection.

After fourteen days post-administration, liver biopsies and blood samples were collected from treated mice to determine percent albumin gene editing, percent AAV donor DNA integration, and human PAH expression levels relative to murine PAH expression levels. To isolate genomic DNA, liver samples were mixed with lysis buffer (15 mg of tissue in 200 μL of lysis buffer+10 μL Proteinase K) and pulverized in a TissueLyser II (Qiagen) using Triple-Pure zirconium beads (Fisher Scientific). Homogenized tissue was then incubated at 56° C. for 30 minutes, and column-purified using a Monarch Genomic DNA Purification kit (New England Biolabs) in accordance with manufacturer's instructions. Final DNA elution was performed in 50 μL of elution buffer (10 mM Tris-Cl, pH 8.5). The concentration and purity of DNA samples were assessed by measuring absorbance at 260 and 280 nm using a Nanodrop device. DNA samples were used for albumin editing and transgene integration quantification. The extent of albumin editing observed for Cas-CLOVER mRNA delivered to the mice was measured by Droplet Digital PCR (ddPCR) using Drop-off assays that contain a fluorescent probe that hybridizes with Cas-CLOVER target site. The extent of transgene integration at the albumin site was measured by ddPCR using a probe-based detection scheme. One primer binds within the PAH transgene was paired with a primer that binds the ALB genomic DNA near the Cas-CLOVER cut site.

To isolate mRNA, liver samples were mixed with lysis buffer (25 mg of tissue in 300 μL of lysis buffer+15 μL Proteinase K) and pulverized in a TissueLyser II (Qiagen) using Triple-Pure zirconium beads (Fisher Scientific). Homogenized tissue was then incubated at RT for 30 minutes, and column-purified using a Quick RNA Miniprep Plus kit (Zymo Research) in accordance with manufacturer's instructions. Final RNA elution was performed in 50 μL of DNase/RNase-free water. The concentration and purity of DNA samples were assessed by measuring absorbance at 260 and 280 nm using a Nanodrop device. RNA samples were used for human PAH mRNA quantification by quantitative real time polymerase chain reaction (RT-qPCR).

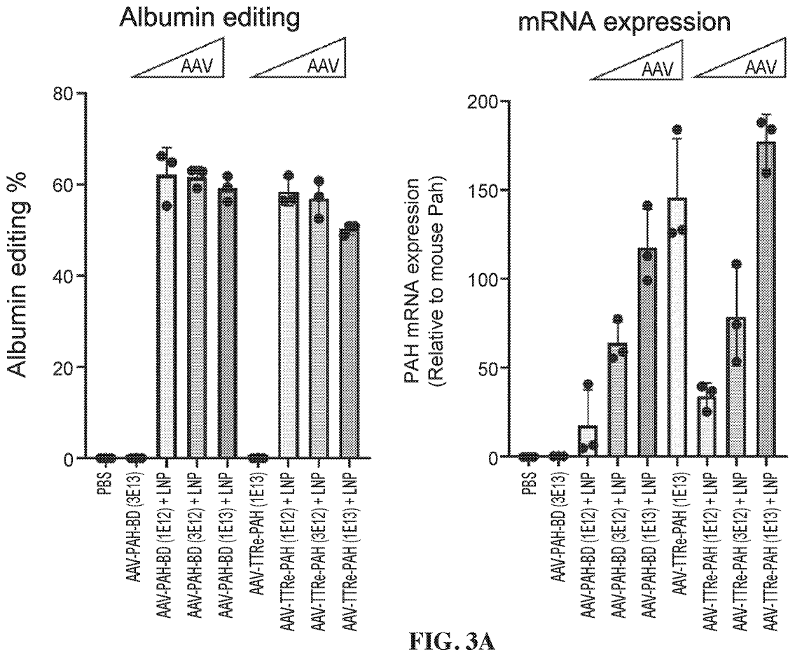

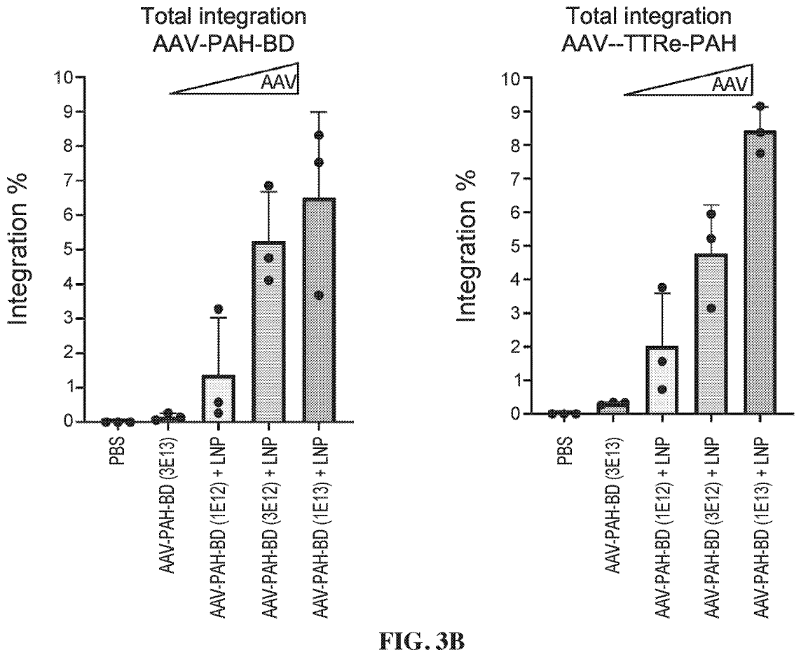

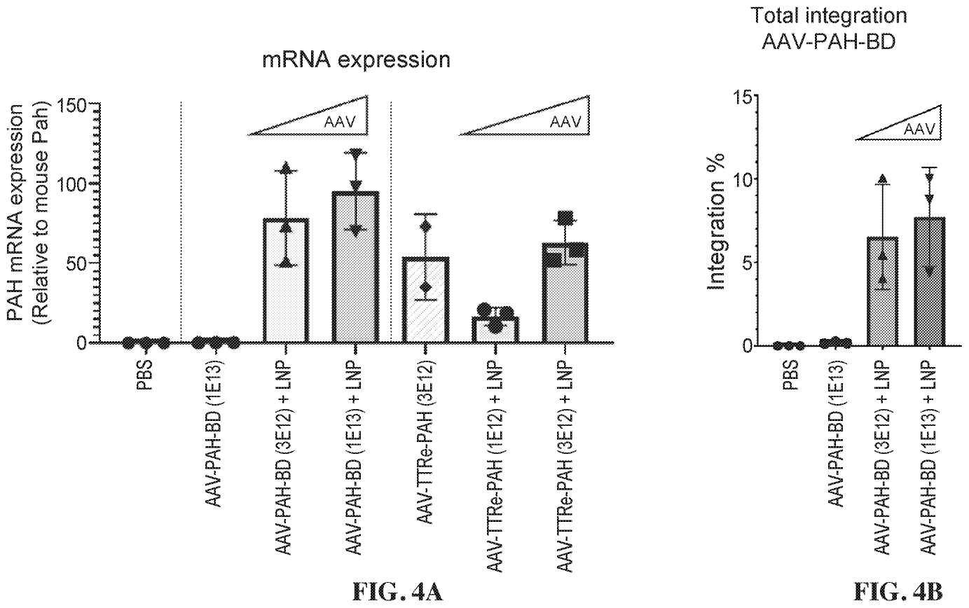

The results are described in FIGS. 3A and 3B. Robust albumin gene editing was observed in the context of the promoterless and the TTRe-PAH AAV donor (1E12; FIG. 3A). Human PAH mRNA expression was dose dependent for both reporter AAV donor polynucleotides (FIG. 4A), as was the total number of integrations per haploid genome (FIG. 3B).

### Example 6—Co-Administration of an AAV Donor Vector Comprising a Codon Optimized and Modified PAH Gene and an LNP Composition Comprising a gRNA Pair and an mRNA Encoding Cas-CLOVER Restores Normal Serum Phenylalanine Levels in BTBR PAH^{enu} Mouse Model

The following is a non-limiting example demonstrating that co-administration of an AAV donor polynucleotides comprising a codon optimized and modified hPAH gene and an LNP composition comprising a gRNA pair and an mRNA encoding Cas-CLOVER restores normal serum phenylalanine levels and normal hypopigmentation in a BTBR PAH^{enu} mouse model.

Adult male (n=1-3/group) BTBR PAH^{enu} mice were administered the following treatments:
Treatment #1: Vehicle;
Treatment #2: Promoterless bidirectional PAH expression AAV viral vector particles (1E13 vg/kg; Example 1B);
Treatment #3: Promoterless bidirectional PAH expression AAV viral vector particles (3E12 vg/kg) in combination with 1.5 mg/kg LNPs encapsulating mRNA encoding Cas-CLOVER and a gRNA pair targeting Intron 3 of the murine albumin gene;
Treatment #4: Promoterless bidirectional PAH expression AAV viral vector particles (1E13 vg/kg) in combination with 1.5 mg/kg LNPs encapsulating mRNA encoding Cas-CLOVER and a gRNA pair targeting Intron 3 of the murine albumin gene;
Treatment #5: TTRe-PAH expression AAV viral vector particles (3E12 vg/kg; Example 1C)
Treatment #6: PAH TTRe expression AAV viral vector particles (3E12 vg/kg) in combination with 1.5 mg/kg LNPs encapsulating mRNA encoding Cas-CLOVER and a gRNA pair targeting intron 3 of the murine albumin gene; and
Treatment #7: PAH TTRe expression AAV viral vector particles (1E13 vg/kg) in combination with 1.5 mg/kg LNPs encapsulating mRNA encoding Cas-CLOVER and a gRNA pair targeting intron 3 of the murine albumin gene. Untreated adult C57BL/6 wild type mice (n=3/group) were followed in parallel as a positive control for measuring normal serum Phe levels.

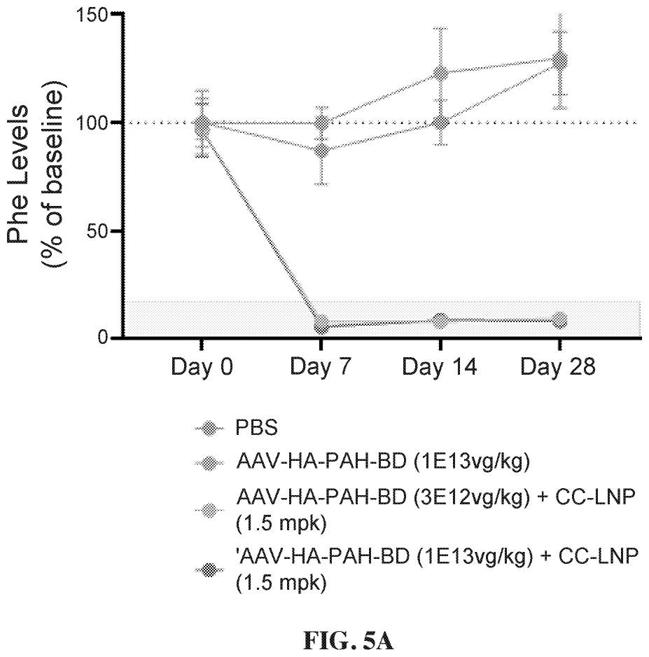

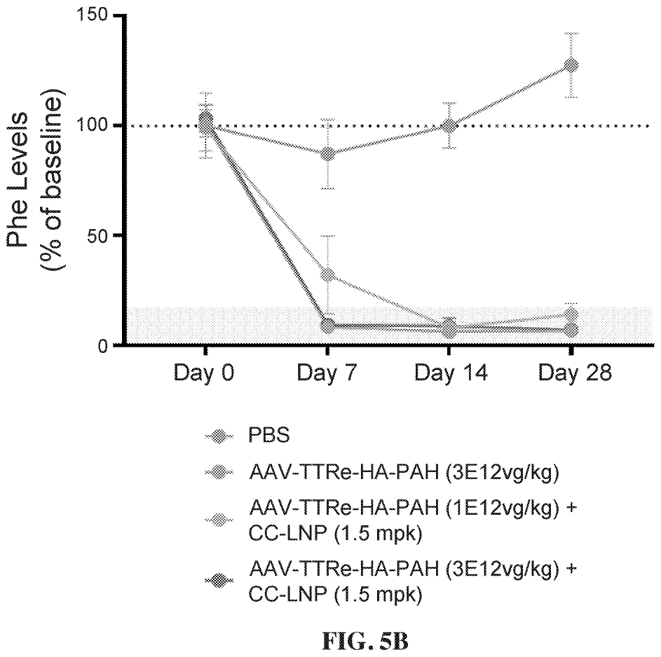

On Days 0, 7, 14 and 28, blood samples were drawn from each group of treated mice and control mice, and serum Phe levels were quantified using a colorimetric readout (Abcam). The results are shown in FIGS. 5A and 5B. As shown in FIG. 5A, diseased mice treated with the promoterless AAV PAH reporter vector and the LNP formulation comprising albumin gRNA pair the mRNA encoding Cas-CLOVER exhibited a rapid and steep decrease in serum Phe levels at Day 7 approaching wild type levels that continued to further decline below baseline at Days 14 & 28 (FIG. 5A).

Similar decreases in serum Phe levels were observed using the AAV-TTRe-PAH reporter vectors in the presence of LNP formulation comprising the albumin gRNA pair and the mRNA encoding Cas-CLOVER (FIG. 5B).

Thus, the co-administration of an AAV donor vector comprising a codon optimized PAH gene of the present disclosure, an LNP composition comprising an albumin intron 3 targeting gRNA pair, and an mRNA encoding Cas-CLOVER in an in vivo PKU disease model reduced serum Phe levels to levels equivalent or below that of wild type mice. These results suggest that the integration of the AAV donor polynucleotide comprising the codon optimized and modified PAH gene by Cas-CLOVER, and subsequent stable expression of PAH in integrated hepatocytes resulted in the reduction of serum Phe levels to normal wild type levels.

What is claimed is:

1. An adeno-associated virus (AAV) donor polynucleotide comprising in the 5′ to 3′ direction:
a) a first AAV ITR sequence;
b) a first targeting sequence homologous to a first region of intron 3 of the albumin gene comprising the sequence of SEQ ID NO: 13;
c) a splice acceptor sequence;
d) a P2A sequence;
e) a sequence encoding a first codon optimized and modified PAH gene comprising the sequence of SEQ ID NO: 25;
f) a DNA spacer sequence;
g) an inverted poly A sequence;
h) an inverted sequence encoding a second codon optimized and modified PAH gene comprising the sequence of SEQ ID NO: 26;
i) an inverted P2A sequence;
j) an inverted splice acceptor sequence;
k) a second targeting sequence homologous to a second region of intron 3 of the albumin gene comprising the sequence of SEQ ID NO: 23; and
l) a second AAV ITR sequence.

2. The AAV donor polynucleotide of claim 1, wherein
the first AAV ITR sequence comprises the sequence of SEQ ID NO: 12;
the splice acceptor sequence comprises the sequence of SEQ ID NO: 14;
the P2A sequence comprises the sequence of SEQ ID NO: 15;
the polyadenylation (poly(A)) sequence comprises the sequence of SEQ ID NO: 17;
the DNA spacer sequence comprises the sequence of SEQ ID NO: 18;
the inverted poly A sequence comprises the sequence of SEQ ID NO: 19;
the inverted P2A sequence comprises the sequence of SEQ ID NO: 21;
the inverted splice acceptor sequence comprises the sequence of SEQ ID NO: 22; and/or
the second AAV ITR sequence comprises the sequence of SEQ ID NO: 24.

3. An adeno-associated virus (AAV) donor polynucleotide comprising in the 5′ to 3′ direction:
a) a first AAV ITR sequence;
b) a first targeting sequence homologous to a first region of intron 3 of the albumin gene comprising the sequence of SEQ ID NO: 13;
c) a TTRe promoter sequence;
d) a splice acceptor sequence;
e) a P2A sequence;
f) a sequence encoding a codon optimized and modified PAH gene comprising the sequence of SEQ ID NO: 25;
g) a 3′UTR sequence;
h) a polyadenylation (poly(A)) sequence;
i) a DNA spacer sequence;
j) a second targeting sequence homologous to a second region of intron 3 of the albumin gene comprising the sequence of SEQ ID NO: 23; and
k) a second AAV ITR sequence.

4. The AAV donor polynucleotide of claim 3, wherein
the first AAV ITR sequence comprises the sequence of SEQ ID NO: 12;
the TTRe promoter sequence comprises the sequence of SEQ ID NO: 27;
the splice acceptor sequence comprises the sequence of SEQ ID NO: 14;
the P2A sequence comprises the sequence of SEQ ID NO: 15;
the 3′ UTR sequence comprises the sequence of SEQ ID NO: 10;
the polyadenylation (poly(A)) sequence comprises the sequence of SEQ ID NO: 17;
the DNA spacer sequence comprises the sequence of SEQ ID NO: 18;
the inverted poly A sequence comprises the sequence of SEQ ID NO: 19; and/or
the second AAV ITR sequence comprises the sequence of SEQ ID NO: 24.

5. The AAV donor polynucleotide of claim 1, wherein the AAV donor polynucleotide comprises the nucleic acid sequence of SEQ ID NO: 8.

6. The AAV donor polynucleotide of claim 3, wherein the AAV donor polynucleotide comprises the nucleic acid sequence of SEQ ID NO: 10.

7. An AAV viral vector comprising the AAV donor polynucleotide of any of the preceding claims.

8. The AAV viral vector of claim 7, wherein the AAV viral vector is an AAV1, AAV2, AAV3, AAV4, AAV5, AAV6, AAV7, AAV8, AAV9, AAV10 or AAV11 viral vector.

9. The vector of claim 7, wherein the AAV viral vector is an AAV8 or AAV9 viral vector.

10. A composition comprising: a) the AAV viral vector of any one of claims 7-9; and b) at least one LNP composition comprising at least one gRNA pair and at least one mRNA molecule encoding Cas-CLOVER.

11. The composition of claim 10, wherein the mRNA molecule comprises a 5′-CAP.

12. The composition of claim 10 or 11, wherein the at least one gRNA pair comprises a left gRNA comprising the sequence of SEQ ID NO: 4 and a right gRNA comprising the sequence of SEQ ID NO: 5.

13. The composition of any one of claims 10-12, wherein the at least one LNP composition comprises:
about 54% of ssPalmO-Ph-P4C2 by moles,
about 35% of cholesterol by moles,
about 10% of DOPC by moles, and
about 1% of DMG-PEG2000 by moles

14. A pharmaceutical composition comprising the AAV viral vector of any one of claims 1-7 or the composition of any one claims 10-13 and a pharmaceutically acceptable carrier.

15. A method of treating phenylketonuria (PKU) in a subject in need thereof comprising administering to the subject: a) at least one therapeutically effective dose of the AAV donor polynucleotide of any one of claims 1-6, the AAV viral vector of any one of claims 7-9, the composition of any one of claims 10-13 or the pharmaceutical composition of claim 14; and b) at least one LNP composition comprising at least one gRNA pair and at least one mRNA molecule encoding Cas-CLOVER.

16. The method of claim 15, wherein the at least one LNP composition comprises:
about 54% of ssPalmO-Ph-P4C2 by moles,
about 35% of cholesterol by moles,
about 5% of DOPC by moles,
about 5% of DSPC by moles, and
about 1% of DMG-PEG2000 by moles.

17. The method of claim 15 or 16, wherein the mRNA molecule comprises a 5′-CAP.

18. The method of claims 15-17, wherein the gRNA pair comprises a left gRNA comprising the sequence of SEQ ID NO: 4 and a right gRNA comprising the sequence of SEQ ID NO: 5.

19. A method of site-specifically integrating a transgene via a viral vector into the genome of at least one cell in a subject comprising administering to the subject a) at least one therapeutically effective dose of the AAV donor polynucleotide of any one of claims 1-6, the AAV viral vector of any one of claims 7-9, the composition of any one of claims 10-13 or the pharmaceutical composition of claim 14; and b) at least one LNP composition comprising at least one gRNA pair and at least one mRNA molecule encoding Cas-CLOVER, wherein the viral vector DNA is integrated into the genome of at least one cell at the genomic cut site generated by Cas-CLOVER.

20. The method of claim 19, wherein the at least one LNP composition comprises:
about 54% of ssPalmO-Ph-P4C2 by moles,
about 35% of cholesterol by moles,
about 5% of DOPC by moles,
about 5% of DSPC by moles, and
about 1% of DMG-PEG2000 by moles.

21. The method of claim 19 or 20, wherein the mRNA molecule further comprises a 5′-CAP.

22. The method of claims 19-21, wherein the gRNA pair comprises a left gRNA comprising the sequence of SEQ ID NO: 4 and a right gRNA comprising the sequence of SEQ ID NO: 5.
